(19) United States
(12) Patent Application Publication
(10) Pub. No.: US 2024/0084282 A1
(43) Pub. Date: Mar. 14, 2024

(54) MODIFIED ARGININE DEIMINASES

(71) Applicant: Polaris Group, George Town (KY)

(72) Inventors: Richard E. Showalter, El Cajon, CA (US); Robert J. Almassy, Vista, CA (US); James A. Thomson, Dallas, TX (US); Wes Sisson, San Diego, CA (US); Wei-Jong Shia, San Diego, CA (US); Li-Chang (Jason) Chen, San Diego, CA (US); Derek Lee, Los Angeles, CA (US)

(21) Appl. No.: 18/241,165

(22) Filed: Aug. 31, 2023

Related U.S. Application Data
(60) Provisional application No. 63/403,348, filed on Sep. 2, 2022.

(51) Int. Cl. C12N 9/78; C12N 9/96

(52) U.S. Cl. CPC C12N 9/78; C12N 9/96; C12Y 305/03006

(57) ABSTRACT

Provided are modified arginine deiminase (ADI) proteins, including ADI proteins that comprise one or more substitutions which increase expression in bacteria as insoluble and refoldable inclusion bodies. Also provided are methods of producing the modified ADI proteins, compositions comprising the ADI proteins, and related methods of treating arginine-dependent and related diseases such as cancer.

This application claims benefit and priority to U.S. Provisional Application No. 63/403,348 filed on Sep. 2, 2022 which is incorporated herein by reference in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on Nov. 27, 2023, is named “2845-8 US.xml” and is 292,501 bytes in size. The sequence listing contained in this XML file is part of the specification and is hereby incorporated by reference herein in its entirety.

### TECHNICAL FIELD

The present disclosure relates generally to modified arginine deiminase (ADI) proteins, including ADI proteins that comprise one or more substitutions which increase expression in bacteria as insoluble and refoldable inclusion bodies, methods of producing the modified ADI proteins, compositions comprising the ADI proteins, and related methods of treating arginine-dependent and related diseases such as cancer.

### BACKGROUND

Arginine depletion therapy can be an effective treatment of certain forms of cancer, among other diseases. For instance, pegylated arginine deiminase (ADI-PEG) can be used to deplete the bloodstream supply of arginine by converting it to citrulline and ammonia. ADI-PEG 20 is an exemplary ADI-PEG that is being investigated in the clinic for tumors deficient in the key enzyme argininosuccinate synthetase-1 (ASS1), which is involved in the conversion of citrulline to arginine. ADI-PEG 20 has been well-tolerated and showed promise in clinical studies (see, e.g., Qiu et al., Cancer Lett. 2015 Aug. 1; 364(1):1-7; Phillips et al., Cancer Res Treat. 2013 December; 45(4):251-62; Feun et al., Curr Pharm Des. 2008; 14(11):1049-57; Feun and Savaraj, Expert Opin Investig Drugs. 2006 July; 15(7):815-22; Feun et al., Curr Opin Clin Nutr Metab Care. 2015 January; 18(1):78-82).

Soluble, active, high level expression of a protein of interest is typically the ultimate goal of biotechnology. However, soluble expression of ADI proteins poses a unique problem. Because some ADIs are soluble, highly-active, and have low Km's for substrate (<10 uM), these enzymes are capable of consuming the intracellular arginine of the host cells in which they are expressed. E. coli can convert the citrulline back into arginine via the argininosuccinate synthetase and argininosuccinate lyase enzymes, but this competitive process puts undue stress on the host cell as evidenced by the low cellular density and low level of protein expression. Also, the constant arginine turnover reduces the tRNA pools for arginine, and thereby limits the levels of overexpressed ADI protein in the host cell.

The expression of recombinant proteins in Escherichia coli often results in the formation of insoluble precipitates known as inclusion bodies (see, e.g., Taylor et al., Bio/Technology 4, 553, 1986). A significant body of research has been developed over the years to try and shift the insoluble expression into soluble expression for functional characterization of the protein of interest up to commercial scale production for industrial, biotechnical or pharmaceutical use (see, e.g., Misawa and Kumagai, Biopolymers 51:297-307, 1999). That said, when all else fails, the inclusion bodies themselves may be used in an attempt to denature and refold the aggregated protein into a properly folded functional state. This process can be very inefficient, difficult, and expensive in time and resources. Each protein is unique and requires the right combination of denaturant, dilution ratios, dilution speed, dialysis conditions, solid phase support systems, temperature of incubation, pH, salt concentration, time of incubation, excipients such as glycerol, glycols, solvents, arginine, detergents, oxidizing and reducing agents, cofactors, etc. in order to achieve a proper renaturation of the protein (see Id., and Middleberg, Trends in Biotech. 20:437-443, 2002).

With these challenges in mind, there is a need to increase the conversion of soluble ADI proteins into insoluble/refoldable inclusion bodies, and thereby minimize the stress of soluble ADI expression on host cells and optimize the recombinant production of ADI proteins. Such an approach could allow ADI proteins to be produced at a scale necessary for drug development, and enhance the commercial potential of ADI proteins as therapeutic enzymes.

### BRIEF SUMMARY

Certain embodiments include an isolated arginine deiminase (ADI), comprising, consisting, or consisting essentially of an amino acid sequence that is at least 90, 95, 96, 97, 98, 99, or 100% identical to an amino sequence selected from Table A1.1 and Table A1.2, excluding SEQ ID NO:1.

In some embodiments, the isolated ADI is recombinantly expressed (or expressible) in a bacterial host cell, optionally E. coli, as insoluble and refoldable inclusion bodies. In some embodiments, at least about 10-100% or at least about 10, 20, 30, 40, 50, 60, 70, 80, 90, or 100% of the ADI is recombinantly expressed (or expressible) in the bacterial host cell as insoluble and refoldable inclusion bodies.

In some embodiments, the isolated ADI has ADI activity under physiological conditions, optionally of temperature, salinity, and pH. In some embodiments, the isolated ADI has at least about 50, 60, 70, 80, 90, 100, 110, or 120% of the ADI activity relative to an isolated ADI that consists of SEQ ID NO:1 (wild-type M. columbinum) under comparable physiological conditions.

Certain isolated ADIs comprise, consist, or consist essentially of an amino acid sequence that is at least 90, 95, 96, 97, 98, 99, or 100% identical to an amino acid sequence selected from SEQ ID NOs:2-178, which retains one or more lysine substitutions selected from K2G, K13E, K63N, K82S, K90T, K90V, K101D, K106L, K108R, K108A, K131R, K170R, K175R, K192V, K192C, K216N, K216V, K229L, K237N, K238N, K240V, K243T, K246E, K248R, K249R, K273R, K275A, K287A, K287Q, K287C, K295A, K2951, K304L, K317R, K326A, and K400A (relative to SEQ ID NO: 1). Certain isolated ADIs retain 1, 2, 3, 4, 5, 6, 7, 8, 9, 10, 11, 12, 13, 14, 15, 16, 17, 18, 19, 20, 21, 22, 23, 24, 25, 26, 27, 28, or 29 of the lysine substitutions selected from K2G, K13E, K63N, K82S, K90T, K90V, K101 D, K106L, K108R, K108A, K131R, K170R, K175R, K192V, K192C, K216N, K216V, K229L, K237N, K238N, K240V, K243T, K246E, K248R, K249R, K273R, K275A, K287Q, K287C, K287A, K295A, K2951, K304L, K317R, K326A, and K400A (relative to SEQ ID NO: 1), for example, all or a portion of the lysine substitutions indicated in Table A2 and Table 3 for the selected sequence.

In some embodiments, the isolated ADI is covalently bonded via a linker to at least one PEG molecule. In some embodiments, the isolated ADI is covalently bonded to about 1 to about 10 PEG molecules. In some embodiments, the isolated ADI is covalently bonded to about 2 to about 8 PEG molecules. In some embodiments, the PEG molecules are straight chain or branch chain PEG molecules. In some embodiments, the PEG has a total weight average molecular weight of from about 1,000 to about 40,000, optionally from about 2,000 to about 20,000, optionally from about 2,000 to about 10,000, optionally about 5,000.

In some embodiments, the linker is a succinyl group, an amide group, an imide group, a carbamate group, an ester group, an epoxy group, a carboxyl group, a hydroxyl group, a carbohydrate, a tyrosine group, a cysteine group, a histidine group, a methylene group, or any combinations thereof. In some embodiments, the source of the succinyl group is a succinimidyl carboxymethyl ester (SCM) or N-hydroxy succinimide (NHS).

Also included are compositions, including therapeutic composition, comprising an isolated arginine deiminase (ADI) described herein, and a pharmaceutically acceptable carrier.

In some embodiments, the composition has a purity of at least about 80%, 85%, 90%, 95%, 98%, or 99% on a protein basis or a weight-weight basis and is substantially aggregate-free. Certain compositions are substantially endotoxin-free.

Also included are methods of treating, ameliorating the symptoms of, or inhibiting the progression of, a cancer in a subject in need thereof, comprising administering to the subject a therapeutic composition or isolated ADI, as described herein.

In some embodiments, the cancer is selected from one or more of hepatocellular carcinoma (HCC), melanoma, metastatic melanoma, pancreatic cancer, prostate cancer, small cell lung cancer, mesothelioma, lymphocytic leukemia, chronic myelogenous leukemia, lymphoma, hepatoma, sarcoma, leukemia, acute myeloid leukemia, relapsed acute myeloid leukemia, B-cell malignancy, breast cancer, ovarian cancer, colorectal cancer, gastric cancer, glioma (e.g., astrocytoma, oligodendroglioma, ependymoma, or a choroid plexus papilloma), glioblastoma multiforme (e.g., giant cell glioblastoma or a gliosarcoma), meningioma, pituitary adenoma, vestibular schwannoma, primary CNS lymphoma, primitive neuroectodermal tumor (medulloblastoma), non-small cell lung cancer (NSCLC), kidney cancer, bladder cancer, uterine cancer, esophageal cancer, brain cancer, head and neck cancers, cervical cancer, testicular cancer, and stomach cancer. In some embodiments, the cancer exhibits reduced expression of argininosuccinate synthetase-1.

Certain embodiments include isolated polynucleotides encoding one or more isolated arginine deiminases (ADIs) described herein, or a vector comprising the polynucleotide. Some polynucleotides or vectors comprise at least one AAC codon that encodes a lysine residue, for example, the K317 residue. Some polynucleotides or vectors comprise at least about 1, 2, 3, 4, 5, 6, 7, 8, 9, 10, 11, 12, 13, 14, 15, 16, 17, 18, 19, or 20 AAG codons that encode a lysine residue.

Also included are recombinant bacterial host cells, for example, E. coli, comprising a polynucleotide or vector described herein.

Some embodiments include methods for recombinantly-producing an isolated arginine deiminase (ADI), comprising
(a) expressing the ADI in a recombinant bacterial host cell described herein, wherein the ADI is expressed in the host cell as insoluble and refoldable inclusion bodies;
(b) removing the insoluble inclusion bodies from the host cell;
(c) purifying the ADI from the insoluble inclusion bodies;
(d) re-folding the ADI in a re-folding buffer; and
(e) purifying the ADI from the re-folding buffer, thereby producing an isolated ADI.

In some embodiments, at least about 10-100% or at least about 10, 20, 30, 40, 50, 60, 70, 80, 90, or 100% of the ADI is expressed in the bacterial host cell as insoluble and refoldable inclusion bodies. Certain embodiments further comprise measuring the ADI activity of the isolated ADI under physiological conditions, optionally of temperature, salinity, and pH, wherein the isolated ADI has ADI activity under the physiological conditions.

In some embodiments, the isolated ADI has at least about 50, 60, 70, 80, 90, 100, 110, or 120% of the ADI activity relative to an isolated ADI that consists of SEQ ID NO:1 (wild-type M. columbinum) under comparable physiological conditions.

Certain embodiments further comprise preparing a therapeutic composition that comprises the isolated ADI, for example, wherein the composition has a purity of at least about 80%, 85%, 90%, 95%, 98%, or 99% on a protein basis or a weight-weight basis, and/or wherein the composition is substantially aggregate-free and substantially endotoxin-free.

### DETAILED DESCRIPTION

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

The practice of the present disclosure will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Current Protocols in Protein Science, Current Protocols in Molecular Biology or Current Protocols in Immunology, John Wiley & Sons, New York, N.Y. (2009); Ausubel et al., Short Protocols in Molecular Biology, 3^{rd} ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art.

Standard techniques may be used for recombinant technology, molecular biological, microbiological, chemical syntheses, chemical analyses, pharmaceutical preparation, formulation, and delivery, and treatment of patients.

For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

As used herein, the term “amino acid” is intended to mean both naturally occurring and non-naturally occurring amino acids as well as amino acid analogs and mimetics. Naturally occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics Arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

“Biocompatible” refers to materials or compounds which are generally not injurious to biological functions and which will not result in any degree of unacceptable toxicity, including allergenic and disease states.

By “coding sequence” is meant any nucleic acid sequence that contributes to the code for the polypeptide product of a gene. By contrast, the term “non-coding sequence” refers to any nucleic acid sequence that does not directly contribute to the code for the polypeptide product of a gene.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “endotoxin free” or “substantially endotoxin free” relates generally to compositions, solvents, and/or vessels that contain at most trace amounts (e.g., amounts having no clinically adverse physiological effects to a subject) of endotoxin, and preferably undetectable amounts of endotoxin. Endotoxins are toxins associated with certain micro-organisms, such as bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art.

Endotoxins can be detected using routine techniques known in the art. For example, the Limulus Amoebocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/mg of active compound. Typically, 1 ng lipopolysaccharide (LPS) corresponds to about 1-10 EU.

The “half-life” of a polypeptide can refer to the time it takes for the polypeptide to lose half of its pharmacologic, physiologic, or other activity, relative to such activity at the time of administration into the serum or tissue of an organism, or relative to any other defined time-point. “Half-life” can also refer to the time it takes for the amount or concentration of a polypeptide to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and ranges in between e.g., 1.5, 1.6, 1.7. 1.8, etc.) the amount produced by no composition (e.g., the absence of agent) or a control composition. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease (including all integers and ranges in between) in the amount produced by no composition (e.g., the absence of an agent) or a control composition. Examples of comparisons and “statistically significant” amounts are described herein.

The terms “polypeptide,” “protein” and “peptide” are used interchangeably and mean a polymer of amino acids not limited to any particular length. The term “enzyme” includes polypeptide or protein catalysts, and with respect to ADI is used interchangeably with protein, polypeptide, or peptide. The terms include modifications such as myristoylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. The terms “polypeptide” and “protein” specifically encompass the ADI enzymes/proteins described herein, or sequences that have deletions from, additions to, and/or substitutions of one or more amino acid of the ADI proteins. In certain embodiments, the polypeptide is a “recombinant” polypeptide, which is produced by recombinant cell that comprises one or more recombinant DNA molecules, which are typically made of heterologous polynucleotide sequences or combinations of polynucleotide sequences that would not otherwise be found in the cell.

The term “isolated” polypeptide or protein referred to herein means that a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or non-covalent interaction) with portions of a protein with which the “isolated protein” is associated in nature, (6) is operably associated (by covalent or non-covalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is substantially free from proteins or polypeptides or other contaminants that are found in its natural environment that would interfere with its use (therapeutic, diagnostic, prophylactic, research or otherwise).

In certain embodiments, the “purity” of any given agent (e.g., ADI or pegylated ADI) in a composition may be specifically defined. For instance, certain compositions may comprise an agent that is at least 70, 75 80, 85, 90, 91, 92, 93, 94, 95, 96, 97, 98, 99, or 100% pure (for example, on a protein basis), including all decimals and ranges in between, as measured, for example, by high performance liquid chromatography (HPLC), a well-known form of column chromatography used frequently in biochemistry and analytical chemistry to separate, identify, and quantify compounds.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997.

The term “solubility” refers to the property of an agent (e.g., ADI or pegylated ADI) provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, pH 7.4, pH 7.6, pH 7.8, or pH 8.0 (e.g., about pH 5-8). In certain embodiments, solubility is measured in water or a physiological buffer such as PBS or NaCl (with or without NaP). In specific embodiments, solubility is measured at relatively lower pH (e.g., pH 6.0) and relatively higher salt (e.g., 500 mM NaCl and 10 mM NaPO₄). In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/ml at room temperature or at 37° C.

A “subject” or a “subject in need thereof” or a “patient” or a “patient in need thereof” includes a mammalian subject such as a human subject.

“Substantially” or “essentially” means nearly totally or completely, for instance, 95%, 96%, 97%, 98%, 99% or greater of some given quantity.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Therapeutic response” refers to improvement of symptoms (whether or not sustained) based on administration of one or more therapeutic agents.

As used herein, “treatment” of a subject (e.g., a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied mutatis mutandis to every other embodiment unless expressly stated otherwise.

Throughout the present disclosure, the following abbreviations may be used: PEG, polyethylene glycol; ADI, arginine deiminase; SS, succinimidyl succinate; SSA, succinimidyl succinimide; SPA, succinimidyl propionate; NHS, N-hydroxy-succinimide; ASS-1, argininosuccinate synthetase-1.

Modified Arginine Deiminases

Certain embodiments relate to isolated arginine deiminases (“ADIs”; or “ADI proteins”, including variants/fragments and pegylated versions thereof), derived from the wild-type ADI from M. columbinum, which are modified to increase expression in bacteria as insoluble and refoldable inclusion bodies. In some instances, an ADI protein is modified to comprise one or more amino acid substitutions of solvent-accessible residues. In some instances, an ADI protein is modified to comprise one or more substitutions of wild-type lysine residues. In some instances, the coding sequence of the ADI protein is modified to comprise one or more non-preferred codons that encode lysine residue(s), for example, AAG codon(s) rather than the preferred AAA codon(s).

In some embodiments, an ADI protein is recombinantly expressed (or expressible) in a bacterial host cell as insoluble and refoldable inclusion bodies. For instance, in some embodiments, at least about 10-100% or 50-100% or at least about 10, 20, 30, 40, 50, 60, 70, 80, 90, or 100% of an ADI protein is recombinantly expressed (or expressible) in the bacterial host cell as insoluble and refoldable inclusion bodies. “Inclusion bodies” refer generally to nuclear or cytoplasmic aggregates of stable substances, usually proteins, and often contain very little host protein, ribosomal components or DNA/RNA fragments. Inclusion bodies can be seen as dense electron-retractile particles of aggregated protein found in both the cytoplasmic and periplasmic spaces of bacteria such as E. coli during high-level expression of heterologous proteins. Inclusion bodies have higher density than many of the cellular components, and thus can be easily separated by high-speed centrifugation after cell disruption. Despite being dense particles, inclusion bodies are highly hydrated and have a porous architecture (see, e.g., Sing and Panda, Journal of Bioscience and Bioengineering, 99: 303-310, 2005). In certain embodiments, inclusion bodies are substantially composed of overexpressed ADI proteins. In particular embodiments, the bacterial host cell is E coil.

In certain embodiments, as noted above, an ADI protein has an “ADI activity”, that is, the ability to convert or metabolize arginine into citrulline and ammonia. ADI or “arginine deiminase” activity can be measured according to routine techniques in the art. For instance, the amount of L-citrulline can be detected by a colorimetric endpoint assay (see, for example, Knipp and Vasak, Analytical Biochem. 286:257-264, 2000) and compared to a standard curve of known amounts of L-citrulline in order to calculate the specific activity of ADI, which can be expressed, for example, as IU/mg of protein. In some embodiments, one IU of ADI enzyme activity is defined as the amount of enzyme that produces 1 μmol of citrulline per minute at the pH and temperature being tested. In some embodiments, an isolated ADI protein has ADI activity under physiological conditions, for example, under physiological conditions of temperature, salinity (for example, solution of a salt or salts that is substantially isotonic with tissue fluids or blood), and pH, for example, about 37° C. and about pH 7.2-7.6, or about pH 7.4. In certain embodiments, an ADI protein described herein has at least about 50, 60, 70, 80, 90, 100, 110, or 120% of the ADI activity relative to an ADI protein that consists of SEQ ID NO:1 (wild-type M. columbinum) under comparable physiological conditions.

The amino acid sequences of exemplary modified ADIs are provided in Table A1.1-A1.2 below, excluding SEQ ID NO:1. Also indicated in Table A1.1 is the % ADI activity relative to the wild-type ADI from M. columbinum (SEQ ID NO:1), as described in the Examples.

TABLE A1.1
Modified ADIs

| | | % ADI | |
|---|---|---|---|
| | | activity | SEQ |
| | | relative | ID |
| Name | Sequence | to SEQ: 1 | NO: |
| M. | SKINVYSEIGELKEVLVHTPGDEIRRISPSRLDELLFSAILEPNEAI | N/A | 1 |
| columbinum | KEHKGFLKILQDKGIKVIQLSDLVAETYTYHATQKEREAFIEKWLDE | | |
| | AEPALTKDIRAKVKSYVLSKEGTPVAMVRIMMAGVSKQELNVESETE | | |
| | LVVDPMPNLYFTRDPFASAGNGISINNMKYVIRKRETIFAEFIFATH | | |
| | PDYKITPHWFDRLDEGNIEGGDVFIYNKDILVIGVSERINKEAILTI | | |
| | AKKIKNNKEAKFKKIVAINVPPMPNLMHLDIWLTMVDKDKFLYSPNM | | |
| | LSVLKVWEIDLSKEIEMVETNKPLADVLESIIGVKPVLIPIAGKGAT | | |
| | QLDIDIETAFDGTNYLTIAPGVVVGYSRNIKTEAALRAAGVTVLSFE | | |
| | GNQLSLGMGSARCMSMPLVREDVK | | |
| McolM1 | SGINVYSEIGELEEVLVHTPGDEIRRISPSRLDELLFSAILEPNEAI | ~17 | 2 |
| Refold | KEHKGFLKILQDKGINVIQLSDEVAETYTYHATQSEREAFIETWLDE | | |
| | AEPALTDDLRALVRSYVISKEGTPVAMVRIMMAGVSRQELNVESETE | | |
| | LVVDPMPNLYFTRDPFASAGNGISLNNMRYVTRRRETIFAEFIFATH | | |
| | PDYVTTPHWFDRLDEGNIEGGDVFIYNNDTLVIGVSERINLEAILTI | | |
| | ANNIVNNTEAEFRRIVAINVPPMPNLMALDIWLTMVDRDAFLYSPNM | | |
| | LSVLQVWEIDLSAEIEMVETNIPLADVLESIIGVRPVLIPIAGAGAT | | |
| | QLDIDIETHEDGTNYLTIAPGVVVGYSRNIKTEAALRAAGVTVLSFE | | |
| | GNQLSLGMGSARCMSMPLVREDVA | | |
| McolM2 | SGINVYSEIGELKEVEVHIPGDEIRRISPSRLDELLFSAILEPNEAI | <80 | 3 |
| Refold | KEHKGFLKILQDKGINVIQLSDLVAETYTYHATQKEREAFIETWLDE | | |
| | AEPALTKDIRALVKSYVLSKEGIPVAMVRIMMAGVSRQELNVESETE | | |
| | LVVDPMPNLYFTRDPFASAGNGISLNNMKYVIRRRETIFAEFIFATH | | |
| | PDYKITPHWFDRLDEGNIEGGDVFIYNNDILVIGVSERINKEAILTI | | |
| | ANKIVNNKEAEFKRIVAINVPPMPNLMHLDTWLTMVDKDAFLYSPNM | | |
| | ISVLKVWEIDLSAEIEMVETNKPLADVLESIIGVRPVLIPIAGKGAT | | |
| | QLDIDIETHFDGTNYLTIAPGVVVGYSRNIKTEAALRAAGVIVISFE | | |
| | GNQLSLGMGSARCMSMPLVREDVA | | |
| McolM3 | SGINVYSEIGELEEVLVHTPGDEIRRISPSRLDELLFSAILEPNEAI | <80 | 4 |
| Refold | KEHKGFLKILQDKGIKVIQLSDLVAETYTYHATQSEREAFIEKWIDE | | |
| | AEPALTDDLRAKVRSYVLSKEGIPVAMVRIMMAGVSKQELNVESETE | | |
| | LVVDPMPNLYFTRDPFASAGNGISLNNMRYVIRKRETIFAEFIFATH | | |
| | PDYVTTPHWEDRLDEGNIEGGDVFIYNKDTLVIGVSERINLEAILTI | | |
| | AKNIKNNTEAKFRKIVAINVPPMPNLMALDIWLTMVDRDKFLYSPNM | | |
| | LSVLQVWEIDISKEIEMVETNIPLADVLESIIGVKPVLIPIAGAGAT | | |
| | QLDIDIETHEDGTNYLTIAPGVVVGYSRNIKTEAALRAAGVIVLSFE | | |
| | GNQLSLGMGSARCMSMPLVREDVK | | |
| McolM4 | SGINVYSEIGELKEVLVHIPGDEIRRISPSRLDELLFSAILEPNEAI | >80 | 5 |
| Soluble | KEHKGFLKILQDKGIKVIQLSDLVAETYTYHATQKEREAFIETWLDE | | |
| | AEPALTKDLRAKVKSYVLSKEGTPVAMVRIMMAGVSRQELNVESETE | | |
| | LVVDPMPNLYFTRDPFASAGNGISLNNMKYVTRKRETIFAEFIFATH | | |
| | PDYKTTPHWFDRLDEGNIEGGDVFIYNNDTLVIGVSERINKEAILTI | | |
| | AKKIVNNKEAKFKRIVAINVPPMPNLMHLDIWLTMVDKDKFLYSPNM | | |
| | LSVLKVWEIDLSAEIEMVETNKPLADVLESIIGVKPVLIPIAGKGAT | | |
| | QLDIDIETHEDGTNYLTIAPGVVVGYSRNIKTEAALRAAGVTVLSFE | | |
| | GNQLSLGMGSARCMSMPLVREDVA | | |
| McolM5 | SGINVYSEIGELKEVLVHIPGDEIRRISPSRLDELLFSAILEPNEAI | <80 | 6 |
| Refold | KEHKGFLKILQDKGINVIQLSDLVAETYTYHATQKEREAFIEKWLDE | | |
| | AEPALTKDIRALVKSYVLSKEGTPVAMVRIMMAGVSKQELNVESETE | | |
| | LVVDPMPNLYFTRDPFASAGNGISINNMKYVIRRRETIFAEFIFATH | | |
| | PDYKITPHWFDRLDEGNIEGGDVFIYNKDILVIGVSERINKEAILTI | | |
| | ANKIKNNKEAEFKKIVAINVPPMPNLMHLDTWLTMVDKDAFLYSPNM | | |
| | LSVLKVWEIDLSKEIEMVETNKPLADVLESIIGVRPVLIPIAGKGAT | | |
| | QLDIDIETAFDGTNYLTIAPGVVVGYSRNIKTEAALRAAGVTVLSFE | | |
| | GNQLSLGMGSARCMSMPLVREDVK | | |
| McolM6 | SGINVYSEIGELEEVLVHTPGDEIRRISPSRLDELLFSAILEPNEAI | <80 | 7 |
| Refold | KEHKGFLKILQDKGIKVIQLSDLVAETYTYHATQKEREAFIEKWLDE | | |
| | AEPALTDDLRAKVKSYVLSKEGIPVAMVRIMMAGVSKQELNVESETE | | |
| | LVVDPMPNLYFTRDPFASAGNGISLNNMRYVTRKRETIFAEFIFATH | | |
| | PDYKITPHWFDRLDEGNIEGGDVFIYNKDTLVIGVSERINLEAILTI | | |
| | AKKIKNNTEAKFKKIVAINVPPMPNLMALDIWLTMVDRDKFLYSPNM | | |
| | LSVLKVWEIDISKEIEMVETNIPLADVLESIIGVKPVLIPIAGKGAT | | |
| | QLDIDIETHEDGTNYLTIAPGVVVGYSRNIKTEAALRAAGVTVLSFE | | |
| | GNQLSLGMGSARCMSMPLVREDVK | | |
| McoLM7 | SGINVYSEIGELKEVLVHIPGDEIRRISPSRLDELLFSAILEPNEAI | >80 | 8 |
| Soluble | KEHKGFLKILQDKGIKVIQLSDLVAETYTYHATQSEREAFIEKWLDE | | |
| | AEPALTKDLRAKVRSYVLSKEGTPVAMVRIMMAGVSKQELNVESETE | | |
| | LVVDPMPNLYFTRDPFASAGNGISLNNMKYVIRKRETIFAEFIFATH | | |
| | PDYVTTPAWEDRLDEGNIEGGDVFIYNKDTLVIGVSERINKEAILTI | | |
| | AKNIKNNKEAKFRKIVAINVPPMPNLMHIDTWLTMVDKDKFLYSPNM | | |
| | LSVLQVWEIDLSKEIEMVETNKPLADVLESIIGVKPVLIPIAGAGAT | | |
| | QLDIDIETHEDGTNYETIAPGVVVGYSRNIKTEAALRAAGVTVLSFE | | |
| | GNQLSLGMGSARCMSMPLVREDVK | | |
| McolM8 | SGINVYSEIGELKEVLVHIPGDEIRRISPSRLDELLFSAILEPNEAI | >80 | 9 |
| Soluble | KEHKGFLKILQDKGIKVIQLSDLVAETYTYHATQSEREAFIETWLDE | | |
| | AEPALTKDLRAKVRSYVLSKEGIPVAMVRIMMAGVSRQELNVESETE | | |
| | LVVDPMPNLYFTRDPFASAGNGISLNNMKYVIRKRETIFAEFIFATH | | |
| | PDYVTTPHWEDRLDEGNIEGGDVFIYNNDILVIGVSERINKEAILTI | | |
| | AKNIVNNKEAKFRRIVAINVPPMPNLMHLDIWLTMVDKDKFLYSPNM | | |
| | ISVLQVWEIDLSAEIEMVETNKPLADVLESIIGVKPVLIPIAGAGAT | | |
| | QLDIDIETHEDGTNYLTIAPGVVVGYSRNIKTEAALRAAGVTVLSFE | | |
| | GNQLSLGMGSARCMSMPLVREDVA | | |
| McolM9 | SGINVYSEIGELKEVLVHIPGDEIRRISPSRLDELLFSAILEPNEAI | >80 | 10 |
| Refold | KEHKGFLKILQDKGIKVIQLSDEVAETYTYHATQSEREAFIETWLDE | | |
| | AEPALTKDLRAKVRSYVISKEGTPVAMVRIMMAGVSRQELNVESETE | | |
| | LVVDPMPNLYFTRDPFASAGNGISLNNMKYVTRRRETIFAEFIFATH | | |
| | PDYVTTPHWFDRLDEGNIEGGDVFIYNNDILVIGVSERINKEAILTI | | |
| | AKNIVNNKEAEFRRIVAINVPPMPNLMHLDIWLTMVDKDKFLYSPNM | | |
| | LSVLQVWEIDLSAEIEMVETNKPLADVLESIIGVRPVLIPIAGAGAT | | |
| | QLDIDIETHEDGTNYLTIAPGVVVGYSRNIKTEAALRAAGVTVLSFE | | |
| | GNQLSLGMGSARCMSMPLVREDVA | | |
| McolM10 | SGINVYSEIGELKEVLVHIPGDEIRRISPSRLDELLFSAILEPNEAI | >80 | 11 |
| Refold | KEHKGFLKILQDKGIKVIQLSDLVAETYTYHATQSEREAFIETWLDE | | |
| | AEPALTKDLRALVRSYVLSKEGIPVAMVRIMMAGVSRQELNVESETE | | |
| | LVVDPMPNLYFTRDPFASAGNGISLNNMKYVTRKRETIFAEFIFATH | | |
| | PDYVTTPHWFDRLDEGNIEGGDVFIYNNDTLVIGVSERINKEAILTI | | |
| | ANNIVNNKEAKFRRIVAINVPPMPNLMHLDTWLTMVDKDAFLYSPNM | | |
| | LSVIQVWEIDLSAEIEMVETNKPLADVLESIIGVKPVLIPIAGAGAT | | |
| | QLDIDIETHEDGTNYLTIAPGVVVGYSRNIKTEAALRAAGVTVLSFE | | |
| | GNQLSLGMGSARCMSMPLVREDVA | | |
| McolM11 | SGINVYSEIGELKEVLVHIPGDEIRRISPSRLDELLFSAILEPNEAI | <80 | 12 |
| Refold | KEHKGELKILQDKGIKVIQLSDIVAETYTYHATQSEREAFIETWLDE | | |
| | AEPALTKDLRAKVRSYVLSKEGTPVAMVRTMMAGVSRQELNVESETE | | |
| | LVVDPMPNLYFTRDPFASAGNGISLNNMRYVIRKRETIFAEFIFATH | | |
| | PDYVTTPHWFDRIDEGNIEGGDVFIYNNDILVIGVSERINKEAILTI | | |
| | AKNIVNNTEAKFRRIVAINVPPMPNLMHLDTWLTMVDKDKFLYSPNM | | |
| | LSVIQVWEIDLSAEIEMVETNLPLADVLESIIGVKPVLIPIAGAGAT | | |
| | QLDIDIETHEDGTNYLTIAPGVVVGYSRNIKTEAALRAAGVTVLSFE | | |
| | GNQLSLGMGSARCMSMPLVREDVA | | |
| McolM12 | SGINVYSEIGELKEVLVHTPGDEIRRISPSRLDELLFSAILEPNEAI | <80 | 13 |
| Refold | KEHKGFLKILQDKGIKVIQLSDLVAETYTYHATQSEREAFIETWLDE | | |
| | AEPALTDDLRAKVRSYVLSKEGIPVAMVRIMMAGVSRQELNVESETE | | |
| | LVVDPMPNLYFTRDPFASAGNGISLNNMKYVIRKRETIFAEFIFATH | | |
| | PDYVTTPHWFDRIDEGNIEGGDVFIYNNDILVIGVSERINLEAILTI | | |
| | AKNIVNNKEAKFRRIVAINVPPMPNLMHLDIWLTMVDRDKFLYSPNM | | |
| | LSVLQVWEIDLSAEIEMVETNKPLADVLESIIGVKPVLIPIAGAGAT | | |
| | QLDIDIETHEDGTNYLTIAPGVVVGYSRNIKTEAALRAAGVTVLSFE | | |
| | GNQLSLGMGSARCMSMPLVREDVA | | |
| McolM29 | SGINVYSEIGELKEVLVHIPGDEIRRISPSRLDELLFSAILEPNEAI | >80 | 14 |
| Refold | KEHKGFLKILQDKGIKVIQLSDLVAETYTYHATQSEREAFIETWLDE | | |
| | AEPALTKDIRAKVRSYVLSKEGIPVAMVRIMMAGVSRQELNVESETE | | |
| | LVVDPMPNLYFTRDPFASAGNGISLNNMKYVIRKRETIFAEFIFATH | | |
| | PDYVTTPHWFDRLDEGNIEGGDVFIYNNDTLVIGVSERINKEAILTI | | |
| | AKNIVNNKEAEFRRIVAINVPPMPNLMHIDTWLTMVDKDKFLYSPNM | | |
| | LSVLQVWEIDLSAEIEMVETNKPLADVLESIIGVRPVLIPIAGAGAT | | |
| | QLDIDIETHEDGTNYLTIAPGVVVGYSRNIKTEAALRAAGVTVLSFE | | |
| | GNQLSLGMGSARCMSMPLVREDVA | | |
| McolM30 | SGINVYSEIGELKEVLVHIPGDEIRRISPSRLDELLFSAILEPNEAI | <80 | 15 |
| Refold | KEHKGELKILQDKGIKVIQLSDIVAETYTYHATQSEREAFIETWLDE | | |
| | AEPALTKDLRAKVRSYVLSKEGIPVAMVRIMMAGVSRQELNVESETE | | |
| | LVVDPMPNLYFTRDPFASAGNGISLNNMKYVIRRRETIFAEFIFATH | | |
| | PDYVTTPHWFDRLDEGNIEGGDVFIYNNDTLVIGVSERINKEAILTI | | |
| | AKNIVNNKEAKFRRIVAINVPPMPNLMALDIWLTMVDKDKFLYSPNM | | |
| | LSVLQVWEIDISAEIEMVETNKPLADVLESIIGVRPVLIPIAGAGAT | | |
| | QLDIDIETHEDGTNYLTIAPGVVVGYSRNIKTEAALRAAGVTVLSFE | | |
| | GNQLSLGMGSARCMSMPLVREDVA | | |
| McoLM31 | SGINVYSEIGELKEVIVHIPGDEIRRISPSRLDELLFSAILEPNEAI | >80 | 16 |
| Refold | KEHKGFLKILQDKGIKVIQLSDLVAETYTYHATQSEREAFIETWLDE | | |
| | AEPALTKDLRAKVRSYVLSKEGTPVAMVRTMMAGVSRQELNVESETE | | |
| | LVVDPMPNLYFTRDPFASAGNGISLNNMKYVIRRRETIFAEFIFATH | | |
| | PDYVTTPHWFDRLDEGNIEGGDVFIYNNDTLVIGVSERINKEAILTI | | |
| | AKNIVNNKEAEFRRIVAINVPPMPNLMHLDIWLTMVDKDKFLYSPNM | | |
| | LSVLQVWEIDLSAEIEMVETNKPLADVLESIIGVKPVLIPIAGAGAT | | |
| | QLDIDIETHEDGTNYLTIAPGVVVGYSRNIKTEAALRAAGVTVLSFE | | |
| | GNQLSLGMGSARCMSMPLVREDVA | | |
| McolM32 | SGINVYSEIGELKEVLVHIPGDEIRRISPSRLDELLFSAILEPNEAI | <80 | 17 |
| Refold | KEHKGFLKILQDKGIKVIQLSDIVAETYTYHATQSEREAFIETWLDE | | |
| | AEPALTKDIRAKVRSYVLSKEGTPVAMVRIMMAGVSRQELNVESETE | | |
| | LVVDPMPNLYFTRDPFASAGNGISINNMKYVIRRRETIFAEFIFATH | | |
| | PDYVITPHWFDRLDEGNIEGGDVFIYNNDILVIGVSERINKEAILTI | | |
| | AKNIVNNKEAKFRRIVAINVPPMPNLMHLDIWLTMVDKDKFLYSPNM | | |
| | LSVLQVWEIDLSAEIEMVETNKPLADVLESIIGVKPVLIPIAGAGAT | | |
| | QLDIDIETAFDGTNYLTIAPGVVVGYSRNIKTEAALRAAGVTVLSFE | | |
| | GNQLSLGMGSARCMSMPLVREDVA | | |
| McolM33 | SGINVYSEIGELKEVLVHIPGDEIRRISPSRLDELLFSAILEPNEAI | >80 | 18 |
| Refold | KEHKGFLKILQDKGIKVIQLSDLVAETYTYHATQSEREAFIETWLDE | | |
| | AEPALTKDLRAKVRSYVISKEGTPVAMVRIMMAGVSRQELNVESETE | | |
| | LVVDPMPNLYFTRDPFASAGNGISLNNMKYVIRKRETIFAEFIFATH | | |
| | PDYVTTPHWFDRLDEGNIEGGDVFIYNNDILVIGVSERINKEAILTI | | |
| | AKNIVNNKEAEFRRIVAINVPPMPNLMALDIWLTMVDKDKFLYSPNM | | |
| | LSVLQVWEIDLSAEIEMVETNKPLADVLESIIGVKPVLIPIAGAGAT | | |
| | QLDIDIETHEDGTNYLTIAPGVVVGYSRNIKTEAALRAAGVTVLSFE | | |
| | GNQLSLGMGSARCMSMPLVREDVA | | |
| McoLM34 | SGINVYSEIGELKEVLVHIPGDEIRRISPSRLDELLFSAILEPNEAI | >80 | 19 |
| Refold | KEHKGFLKILQDKGIKVIQLSDLVAETYTYHATQSEREAFIETWLDE | | |
| | AEPALTKDERAKVRSYVLSKEGTPVAMVRIMMAGVSRQELNVESETE | | |
| | LVVDPMPNLYFTRDPFASAGNGISLNNMKYVTRKRETIFAEFIFATH | | |
| | PDYVTTPHWEDRLDEGNIEGGDVFIYNNDTLVIGVSERINKEAILTI | | |
| | AKNIVNNKEAKFRRIVAINVPPMPNLMHIDTWLTMVDKDKFLYSPNM | | |
| | LSVLQVWEIDLSAEIEMVETNKPLADVLESIIGVRPVLIPIAGAGAT | | |
| | QLDIDIETHEDGTNYLTIAPGVVVGYSRNIKTEAALRAAGVTVLSFE | | |
| | GNQLSLGMGSARCMSMPLVREDVA | | |
| McolM35 | SGINVYSEIGELKEVLVHIPGDEIRRISPSRLDELLFSAILEPNEAI | >80 | 20 |
| Refold | KEHKGFLKILQDKGIKVIQLSDLVAETYTYHATQSEREAFIETWLDE | | |
| | AEPALTKDLRAKVRSYVLSKEGTPVAMVRIMMAGVSRQELNVESETE | | |
| | LVVDPMPNLYFTRDPFASAGNGISLNNMKYVIRKRETIFAEFIFATH | | |
| | PDYCTTPHWEDRLDEGNIEGGDVEIYNNDILVIGVSERINKEAILTI | | |
| | AKNIVNNKEAKFRRIVAINVPPMPNLMHLDIWLTMVDKDKFLYSPNM | | |
| | LSVLQVWEIDLSAEIEMVETNKPLADVLESIIGVKPVLIPIAGAGAT | | |
| | QLDIDIETAFDGTNYLTIAPGVVVGYSRNIKTEAALRAAGVTVLSFE | | |
| | GNQLSLGMGSARCMSMPLVREDVA | | |
| McolM36 | SGINVYSEIGELKEVLVHIPGDEIRRISPSRLDELLFSAILEPNEAI | >80 | 21 |
| Refold | KEHKGFLKILQDKGIKVIQLSDEVAETYTYHATQSEREAFIETWLDE | | |
| | AEPALTKDLRAKVRSYVISKEGTPVAMVRIMMAGVSRQELNVESETE | | |
| | LVVDPMPNLYFTRDPFASAGNGISLNNMKYVTRKRETIFAEFIFATH | | |
| | PDYVTTPHWFDRLDEGNIEGGDVFIYNNDILVIGVSERINKEAILTI | | |
| | AKNIVNNKEAKFRRIVAINVPPMPNLMALDIWLTMVDKDKFLYSPNM | | |
| | LSVLCVWEIDLSAEIEMVETNKPLADVLESIIGVKPVLIPIAGAGAT | | |
| | QLDIDIETHEDGTNYLTIAPGVVVGYSRNIKTEAALRAAGVTVLSFE | | |
| | GNQLSLGMGSARCMSMPLVREDVA | | |
| McolM39 | SGINVYSEIGELKEVLVHIPGDEIRRISPSRLDELLFSAILEPNEAI | >80 | 22 |
| Refold | KEAKGFLKILQDKGIKVIQLSDIVAETYTYHATQSEREAFIETWLDE | | |
| | AEPALTKDLRAKVRSYVLSKEGTPVAMVRIMMAGVSRQELNVESETE | | |
| | LVVDPMPNLYFTRDPFASAGNGISLNNMKYVTRKRETIFAEFIFATH | | |
| | PDYCTTPHWFDRLDEGNIEGGDVFIYNNDTLVIGVSERINKEAILTI | | |
| | AKNIVNNKEAKFRRIVAINVPPMPNLMHLDTWLTMVDKDKFLYSPNM | | |
| | LSVLCVWEIDLSAEIEMVETNKPLADVLESIIGVKPVLIPIAGAGAT | | |
| | QLDIDIETHEDGTNYLTIAPGVVVGYSRNIKTEAALRAAGVTVLSFE | | |
| | GNQLSLGMGSARCMSMPLVREDVA | | |
| McolM46 | SGINVYSEIGELKEVLVHTPGDEIRRISPSRLDELLFSAILEPNEAI | >80 | 23 |
| Refold | KEHKGFLKILQDKGIKVIQLSDLVAETYTYHATQSEREAFIETWIDE | | |
| | AEPALTKDLRAKVRSYVLSKEGIPVAMVRIMMAGVSRQELNVESETE | | |
| | LVVDPMPNLYFTRDPFASAGNGISLNNMKYVIRKRETIFAEFIFATH | | |
| | PDYVTTPHWFDRLDEGNIEGGDVFIYNNDILVIGVSERINKEAILTI | | |
| | AKNIVNNKEAKFRRIVAINVPPMPNLMHLDIWLTMVDKDKFLYSPNM | | |
| | ISVLQVWEIDLSAEIEMVEINKPLADVLESIIGVKPVLIPIAGAGAT | | |
| | QLDIDIETHEDGTNYLTIAPGVVVGYSRNIKTEAALRAAGVTVLSFE | | |
| | GNQLSLGMGSARCMSMPLVREDVA | | |
| McolM56 | SRINVYSEIGELKEVIVHIPGDEIRRISPSRLDELLFSAILEPNEAI | ND | 24 |
| Soluble | KEHKGFLKILQDKGIKVIQLSDEVAETYTYHATQREREAFIERWLDE | | |
| | AEPALTKDLRAKVRSYVLSKEGTPVAMVRTMMAGVSRQELNVESETE | | |
| | LVVDPMPNLYFTRDPFASAGNGISLNNMKYVTRKRETIFAEFIFATH | | |
| | PDYRTTPHWFDRLDEGNIEGGDVFIYNRDTLVIGVSERINKEAILTI | | |
| | AKRIRNNKEAEFRRIVAINVPPMPNLMHLDTWLTMVDKDKFLYSPNM | | |
| | LSVLRVWEIDLSREIEMVETNKPLADVLESIIGVKPVLIPIAGRGAT | | |
| | QLDIDIETHEDGTNYLTIAPGVVVGYSRNIKTEAALRAAGVTVLSFE | | |
| | GNQLSLGMGSARCMSMPLVREDVR | | |
| McolM59 | SKINVYSEIGELKEVLVHIPGDEIRRISPSRLDELLFSAILEPNEAI | >80 | 25 |
| Refold | KEHKGFLKILQDKGIKVIQLSDIVAETYTYHATQSEREAFIETWLDE | | |
| | AEPALTKDLRAKVRSYVLSKEGTPVAMVRIMMAGVSRQELNVESETE | | |
| | LVVDPMPNLYFTRDPFASAGNGISLNNMKYVTRKRETIFAEFIFATH | | |
| | PDYVTTPHWFDRLDEGNIEGGDVFIYNNDILVIGVSERINKEAILTI | | |
| | AKNIVNNKEAEFRRIVAINVPPMPNLMHLDTWLTMVDKDKFLYSPNM | | |
| | LSVLQVWEIDLSAEIEMVETNKPLADVLESIIGVKPVLIPIAGAGAT | | |
| | QLDIDIETHFDGTNYLTIAPGVVVGYSRNIKTEAALRAAGVTVLSFE | | |
| | GNQLSLGMGSARCMSMPLVREDVK | | |
| McolM60 | SKINVYSEIGELKEVLVHIPGDEIRRISPSRLDELLFSAILEPNEAI | >80 | 26 |
| Refold | KEHKGFLKILQDKGIKVIQLSDLVAETYTYHATQSEREAFIETWIDE | | |
| | AEPALTKDLRAKVRSYVLSKEGIPVAMVRIMMAGVSRQELNVESETE | | |
| | LVVDPMPNLYFTRDPFASAGNGISLNNMKYVIRKRETIFAEFIFATH | | |
| | PDYVTTPHWFDRLDEGNIEGGDVFIYNNDTLVIGVSERINKEAILTI | | |
| | AKNIVNNKEAEFRRIVAINVPPMPNLMALDIWLIMVDKDKFLYSPNM | | |
| | LSVIQVWEIDLSAEIEMVEINKPLADVLESIIGVKPVLIPIAGKGAT | | |
| | QLDIDIETHEDGTNYLTIAPGVVVGYSRNIKTEAALRAAGVTVLSFE | | |
| | GNQLSLGMGSARCMSMPLVREDVK | | |
| McolM61 | SKINVYSEIGELKEVIVHIPGDEIRRISPSRLDELLFSAILEPNEAI | >80 | 27 |
| 50/50 | KEHKGFLKILQDKGIKVIQLSDLVAETYTYHATQSEREAFIETWLDE | | |
| | AEPALTKDLRAKVRSYVLSKEGTPVAMVRIMMAGVSRQELNVESETE | | |
| | LVVDPMPNLYFTRDPFASAGNGISLNNMKYVTRKRETIFAEFIFATH | | |
| | PDYVTTPHWFDRLDEGNIEGGDVFIYNNDILVIGVSERINKEAILTI | | |
| | AKNIVNNKEAEFRRIVAINVPPMPNLMHLDTWLTMVDKDKFLYSPNM | | |
| | LSVLQVWEIDLSKEIEMVETNKPLADVLESIIGVKPVLIPIAGKGAT | | |
| | QLDIDIETHEDGTNYLTIAPGVVVGYSRNIKTEAALRAAGVTVLSFE | | |
| | GNQLSLGMGSARCMSMPLVREDVK | | |
| McolM62 | SKINVYSEIGELKEVLVHIPGDEIRRISPSRLDELLFSAILEPNEAI | ND | 28 |
| Soluble | KEHKGFLKILQDKGIKVIQLSDIVAETYTYHATQSEREAFIETWLDE | | |
| | AEPALTKDLRAKVRSYVLSKEGTPVAMVRIMMAGVSRQELNVESETE | | |
| | LVVDPMPNLYFTRDPFASAGNGISINNMKYVIRKRETIFAEFIFATH | | |
| | PDYVTTPHWFDRLDEGNIEGGDVFIYNNDILVIGVSERINKEAILTI | | |
| | AKNIVNNKEAEFRRIVAINVPPMPNLMHLDTWLTMVDKDKFLYSPNM | | |
| | LSVLKVWEIDLSKEIEMVETNKPLADVLESIIGVKPVLIPIAGKGAT | | |
| | QLDIDIETAFDGTNYLTIAPGVVVGYSRNIKTEAALRAAGVTVLSFE | | |
| | GNQLSLGMGSARCMSMPLVREDVK | | |
| McolM63 | SKINVYSEIGELKEVLVHIPGDEIRRISPSRLDELLFSAILEPNEAI | ND | 29 |
| Soluble | KEHKGFLKILQDKGIKVIQLSDLVAETYTYHATQKEREAFIETWLDE | | |
| | AEPALTKDLRAKVRSYVLSKEGIPVAMVRIMMAGVSRQELNVESETE | | |
| | LVVDPMPNLYFTRDPFASAGNGISLNNMKYVTRKRETIFAEFIFATH | | |
| | PDYVTTPHWFDRLDEGNIEGGDVFIYNNDTLVIGVSERINKEAILTI | | |
| | AKNIVNNKEAEFRRIVAINVPPMPNLMALDIWLTMVDKDKFLYSPNM | | |
| | LSVLKVWEIDISKEIEMVETNKPLADVLESIIGVKPVLIPIAGKGAT | | |
| | QLDIDIETHEDGTNYLTIAPGVVVGYSRNIKTEAALRAAGVIVLSFE | | |
| | GNQLSLGMGSARCMSMPLVREDVK | | |
| McolM64 | SKINVYSEIGELKEVLVHIPGDEIRRISPSRLDELLFSAILEPNEAI | TBD | 30 |
| | KEHKGFLKILQDKGIKVIQLSDLVAETYTYHATQKEREAFIETWLDE | | |
| | AEPALTKDLRAKVRSYVLSKEGTPVAMVRIMMAGVSRQELNVESETE | | |
| | LVVDPMPNLYFTRDPFASAGNGISLNNMKYVTRKRETIFAEFIFATH | | |
| | PDYVTTPAWFDRLDEGNIEGGDVFIYNNDTLVIGVSERINKEAILTI | | |
| | AKNIVNNKEAEFRRIVAINVPPMPNLMHIDTWLTMVDKDKFLYSPNM | | |
| | LSVLQVWEIDLSAEIEMVETNKPLADVLESIIGVKPVLIPIAGKGAT | | |
| | QLDIDIETHEDGTNYLTIAPGVVVGYSRNIKTEAALRAAGVTVLSFE | | |
| | GNQLSLGMGSARCMSMPLVREDVK | | |
| McolM65 | SKINVYSEIGELKEVLVHIPGDEIRRISPSRLDELLFSAILEPNEAI | TBD | 31 |
| | KEHKGFLKILQDKGIKVIQLSDLVAETYTYHATQSEREAFIEKWLDE | | |
| | AEPALTKDLRAKVRSYVLSKEGTPVAMVRIMMAGVSRQELNVESETE | | |
| | LVVDPMPNLYFTRDPFASAGNGISINNMKYVIRKRETIFAEFIFATH | | |
| | PDYVTTPHWFDRLDEGNIEGGDVFIYNNDILVIGVSERINKEAILTI | | |
| | AKNIVNNKEAEFRRIVAINVPPMPNLMHLDTWLTMVDKDKFLYSPNM | | |
| | LSVLQVWEIDLSAEIEMVETNKPLADVLESIIGVKPVLIPIAGKGAT | | |
| | QLDIDIETAFDGTNYLTIAPGVVVGYSRNIKTEAALRAAGVTVLSFE | | |
| | GNQLSLGMGSARCMSMPLVREDVK | | |
| McolM66 | SKINVYSEIGELKEVLVHTPGDEIRRISPSRLDELLFSAILEPNEAI | TBD | 32 |
| | KEHKGFLKILQDKGIKVIQLSDLVAETYTYHATQSEREAFIETWIDE | | |
| | AEPALTKDLRAKVKSYVISKEGTPVAMVRIMMAGVSRQELNVESETE | | |
| | LVVDPMPNLYFTRDPFASAGNGISLNNMKYVTRKRETIFAEFIFATH | | |
| | PDYVTTPHWFDRLDEGNIEGGDVFIYNNDTLVIGVSERINKEAILTI | | |
| | AKNIVNNKEAEFRRIVAINVPPMPNLMALDIWLTMVDKDKFLYSPNM | | |
| | LSVLQVWEIDISAEIEMVETNKPLADVLESIIGVKPVLIPIAGKGAT | | |
| | QLDIDIETHFDGINYLTIAPGVVVGYSRNIKTEAALRAAGVIVLSFE | | |
| | GNQLSLGMGSARCMSMPLVREDVK | | |
| McolM67 | SKINVYSEIGELKEVLVHIPGDEIRRISPSRLDELLESAILEPNEAI | TBD | 33 |
| | KEHKGFLKILQDKGIKVIQLSDLVAETYTYHATQSEREAFIETWLDE | | |
| | AEPALTKDLRAKVRSYVLSKEGTPVAMVRIMMAGVSKQELNVESETE | | |
| | LVVDPMPNLYFTRDPFASAGNGISLNNMKYVTRKRETIFAEFIFATH | | |
| | PDYVTTPHWEDRLDEGNIEGGDVFIYNNDTLVIGVSERINKEAILTI | | |
| | AKNIVNNKEAEFRRIVAINVPPMPNLMHIDTWLTMVDKDKFLYSPNM | | |
| | LSVLQVWEIDLSAEIEMVETNKPLADVLESIIGVKPVLIPIAGKGAT | | |
| | QLDIDIETHEDGTNYLTIAPGVVVGYSRNIKTEAALRAAGVTVLSFE | | |
| | GNQLSLGMGSARCMSMPLVREDVK | | |
| McolM68 | SKINVYSEIGELKEVLVHIPGDEIRRISPSRLDELLFSAILEPNEAI | TBD | 34 |
| | KEHKGFLKILQDKGIKVIQLSDIVAETYTYHATQSEREAFIETWLDE | | |
| | AEPALTKDIRAKVRSYVLSKEGTPVAMVRIMMAGVSRQELNVESETE | | |
| | LVVDPMPNLYFTRDPFASAGNGISLNNMKYVTRKRETIFAEFIFATH | | |
| | PDYKITPHWFDRLDEGNIEGGDVFIYNNDILVIGVSERINKEAILTI | | |
| | AKNIVNNKEAEFRRIVAINVPPMPNLMHLDTWLTMVDKDKFLYSPNM | | |
| | LSVLQVWEIDLSAEIEMVETNKPLADVLESIIGVKPVLIPIAGKGAT | | |
| | QLDIDIETHEDGTNYLTIAPGVVVGYSRNIKTEAALRAAGVTVISFE | | |
| | GNQLSLGMGSARCMSMPLVREDVK | | |
| McolM69 | SKINVYSEIGELKEVLVHTPGDEIRRISPSRLDELLFSAILEPNEAI | TBD | 35 |
| | KEHKGFLKILQDKGIKVIQLSDLVAETYTYHATQSEREAFIETWLDE | | |
| | AEPALTKDLRAKVRSYVLSKEGIPVAMVRIMMAGVSRQELNVESETE | | |
| | LVVDPMPNLYFTRDPFASAGNGISLNNMKYVTRKRETIFAEFIFATH | | |
| | PDYVTTPHWFDRLDEGNIEGGDVFIYNKDILVIGVSERINKEAILTI | | |
| | AKNIVNNKEAEFRRIVAINVPPMPNLMALDIWLIMVDKDKFLYSPNM | | |
| | LSVLQVWEIDISAEIEMVETNKPLADVLESIIGVKPVLIPIAGKGAT | | |
| | QLDIDIETHEDGTNYLTIAPGVVVGYSRNIKTEAALRAAGVIVLSFE | | |
| | GNQLSLGMGSARCMSMPLVREDVK | | |
| McolM70 | SKINVYSEIGELKEVLVHIPGDEIRRISPSRLDELLFSAILEPNEAI | TBD | 36 |
| | KEHKGFLKILQDKGIKVIQLSDLVAETYTYHATQSEREAFIETWLDE | | |
| | AEPALTKDLRAKVRSYVLSKEGTPVAMVRTMMAGVSRQELNVESETE | | |
| | LVVDPMPNLYFTRDPFASAGNGISLNNMKYVTRKRETIFAEFIFATH | | |
| | PDYVTTPHWFDRLDEGNIEGGDVFIYNNDTLVIGVSERINKEAILTI | | |
| | AKKIVNNKEAEFRRIVAINVPPMPNLMHLDTWLTMVDKDKFLYSPNM | | |
| | LSVLQVWEIDLSAEIEMVETNKPLADVLESIIGVKPVLIPIAGKGAT | | |
| | QLDIDIETHEDGTNYETIAPGVVVGYSRNIKTEAALRAAGVTVLSFE | | |
| | GNQLSLGMGSARCMSMPLVREDVK | | |
| McolM71 | SKINVYSEIGELKEVEVHIPGDEIRRISPSRLDELLFSAILEPNEAI | TBD | 37 |
| | KEHKGFLKILQDKGIKVIQLSDLVAETYTYHATQSEREAFIETWLDE | | |
| | AEPALTKDIRAKVRSYVLSKEGTPVAMVRIMMAGVSRQELNVESETE | | |
| | LVVDPMPNLYFTRDPFASAGNGISINNMKYVIRKRETIFAEFIFATH | | |
| | PDYVTTPHWFDRLDEGNIEGGDVFIYNNDILVIGVSERINKEAILTI | | |
| | AKNIKNNKEAEFRRIVAINVPPMPNLMHLDTWLTMVDKDKFLYSPNM | | |
| | LSVLQVWEIDLSAEIEMVETNKPLADVLESIIGVKPVLIPIAGKGAT | | |
| | QLDIDIETAFDGTNYLTIAPGVVVGYSRNIKTEAALRAAGVTVLSFE | | |
| | GNQLSLGMGSARCMSMPLVREDVK | | |
| McolM72 | SKINVYSEIGELKEVIVHTPGDEIRRISPSRLDELLFSAILEPNEAI | TBD | 38 |
| | KEHKGFLKILQDKGIKVIQLSDLVAETYTYHATQSEREAFIETWLDE | | |
| | AEPALTKDLRAKVRSYVLSKEGTPVAMVRIMMAGVSRQELNVESETE | | |
| | LVVDPMPNLYFTRDPFASAGNGISLNNMKYVTRKRETIFAEFIFATH | | |
| | PDYVTTPHWFDRLDEGNIEGGDVFIYNNDTLVIGVSERINKEAILTI | | |
| | AKNIVNNKEAEFKRIVAINVPPMPNLMALDIWLTMVDKDKFLYSPNM | | |
| | LSVLQVWEIDISAEIEMVETNKPLADVLESIIGVKPVLIPIAGKGAT | | |
| | QLDIDIETHEDGTNYLTIAPGVVVGYSRNIKTEAALRAAGVTVLSFE | | |
| | GNQLSLGMGSARCMSMPLVREDVK | | |
| McoLM73 | SKINVYSEIGELKEVLVHIPGDEIRRISPSRLDELLESAILEPNEAI | TBD | 39 |
| | KEHKGFLKILQDKGIKVIQLSDLVAETYTYHATQSEREAFIETWLDE | | |
| | AEPALTKDLRAKVRSYVLSKEGTPVAMVRIMMAGVSRQELNVESETE | | |
| | LVVDPMPNLYFTRDPFASAGNGISLNNMKYVTRKRETIFAEFIFATH | | |
| | PDYVTTPAWFDRLDEGNIEGGDVFIYNNDTLVIGVSERINKEAILTI | | |
| | AKNIVNNKEAEFRKIVAINVPPMPNLMHLDIWLTMVDKDKFLYSPNM | | |
| | LSVLQVWEIDLSAEIEMVETNKPLADVLESIIGVKPVLIPIAGKGAT | | |
| | QLDIDIETHEDGTNYLTIAPGVVVGYSRNIKTEAALRAAGVTVLSFE | | |
| | GNQLSLGMGSARCMSMPLVREDVK | | |
| McolM74 | SKINVYSEIGELKEVLVHIPGDEIRRISPSRLDELLFSAILEPNEAI | TBD | 40 |
| | KEHKGFLKILQDKGIKVIQLSDIVAETYTYHATQSEREAFIETWLDE | | |
| | AEPALTKDLRAKVRSYVLSKEGIPVAMVRIMMAGVSRQELNVESETE | | |
| | LVVDPMPNLYFTRDPFASAGNGISLNNMKYVIRKRETIFAEFIFATH | | |
| | PDYVTTPHWFDRLDEGNIEGGDVFIYNNDILVIGVSERINKEAILTI | | |
| | AKNIVNNKEAEFRRIVAINVPPMPNLMHLDTWLTMVDKDKFLYSPNM | | |
| | ISVLKVWEIDLSAEIEMVETNKPLADVLESIIGVKPVLIPIAGKGAT | | |
| | QLDIDIETHEDGTNYLTIAPGVVVGYSRNIK | | |

TABLE A1.2

| | |
|---|---|
| McolM75 | SKINVYSEIGELKEVLVHIPGDEIRRISPSRLDELLFSAILEPNEAIKEHKGFLKILQD |
| SEQ ID NO: 41 | KGIKVIQLSDLVAETYTYHATQKEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVITPHWFDRLDEGNIEGGDVFIYNNDTLVIGVSERINKEAILTIA |
| | KKIVNNKEAEFRRIVAINVPPMPNLMHLDTWLTMVDKDKFLYSPNMLSVLQVWEIDLSA |
| | EIEMVEINKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM76 | SKINVYSEIGELKEVLVHTPGDEIRRISPSRLDELLFSAILEPNEAIKEHKGFLKILQD |
| SEQ ID NO: 42 | KGIKVIQLSDLVAETYTYHATQSEREAFIETWLDEAEPALTKDLRAKVRSYVLSKEGTP |
| | VAMVRTMMAGVSKQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KKIVNNKEAEFRRIVAINVPPMPNLMHLDTWLIMVDKDKFLYSPNMLSVLQVWEIDLSA |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM77 | SKINVYSEIGELKEVLVHIPGDEIRRISPSRLDELLFSAILEPNEAIKEHKGFLKILQD |
| SEQ ID NO: 43 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KKIVNNKEAKFRRIVAINVPPMPNLMHLDTWLTMVDKDKFLYSPNMLSVLQVWEIDLSA |
| | EIEMVETNKPLADVLESIIGVKPVLIPIAGKGATQLDIDIETHFDGINYLTIAPGVVVG |
| | YSRNIKTEAALRAAGVTVLSFEGNQLSLGMGSARCMSMPLVREDVK |
| McolM78 | SKINVYSEIGELKEVLVHTPGDEIRRISPSRLDELLFSAILEPNEAIKEHKGFLKILQD |
| SEQ ID NO: 44 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KKIVNNKEAEFKRIVAINVPPMPNLMHLDTWLTMVDKDKFLYSPNMLSVLQVWEIDLSA |
| | EIEMVETNKPLADVLESIIGVKPVLIPIAGKGATQLDIDIETHFDGINYLTIAPGVVVG |
| | YSRNIKTEAALRAAGVTVLSFEGNQLSLGMGSARCMSMPLVREDVK |
| McolM79 | SKINVYSEIGELKEVLVHTPGDEIRRISPSRLDELLFSAILEPNEAIKEHKGFLKILQD |
| SEQ ID NO: 45 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTIPHWFDRLDEGNIEGGDVFIYNNDILVIGVSERINKEAILTIA |
| | KKIVNNKEAEFRKIVAINVPPMPNLMHLDTWLTMVDKDKFLYSPNMLSVLQVWEIDLSA |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM80 | SKINVYSEIGELKEVLVHTPGDEIRRISPSRLDELLFSAILEPNEAIKEHKGFLKILQD |
| SEQ ID NO: 46 | KGIKVIQLSDLVAETYTYHATQSEREAFIETWLDEAEPALIKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDILVIGVSERINKEAILTIA |
| | KKIVNNKEAEFRRIVAINVPPMPNLMHLDTWLTMVDKDKFLYSPNMLSVLQVWEIDLSK |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM81 | SKINVYSEIGELKEVLVHTPGDEIRRISPSRLDELLFSAILEPNEAIKEHKGFLKILQD |
| SEQ ID NO: 47 | KGIKVIQLSDLVAETYTYHATQKEREAFIETWLDEAEPALTKDLRAKVRSYVLSKEGTP |
| | VAMVRTMMAGVSKQELNVESETELVVDPMPNLYFTRDPFASAGNGISLNNMKYVTRKRE |
| | TIFAEFIFATHPDYVITPHWFDRLDEGNIEGGDVFIYNNDILVIGVSERINKEAILTIA |
| | KKIVNNKEAEFRRIVAINVPPMPNLMHLDTWLTMVDKDKFLYSPNMLSVLQVWEIDLSA |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM82 | SKINVYSEIGELKEVLVHTPGDEIRRISPSRLDELLFSAILEPNEAIKEHKGFLKILQD |
| SEQ ID NO: 48 | KGIKVIQLSDLVAETYTYHATQSEREAFIETWLDEAEPALTKDLRAKVRSYVLSKEGTP |
| | VAMVRTMMAGVSKQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KKIVNNKEAKFRRIVAINVPPMPNLMHLDTWLIMVDKDKFLYSPNMLSVLQVWEIDLSA |
| | EIEMVEINKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM83 | SKINVYSEIGELKEVLVHTPGDEIRRISPSRLDELLFSAILEPNEAIKEHKGFLKILQD |
| SEQ ID NO: 49 | KGIKVIQLSDLVAETYTYHATQKEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVITPHWEDRLDEGNIEGGDVFIYNNDILVIGVSERINKEAILTIA |
| | KKIVNNKEAKFRRIVAINVPPMPNLMHLDTWLTMVDKDKFLYSPNMLSVLQVWEIDLSA |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM84 | SKINVYSEIGELKEVLVHTPGDEIRRISPSRLDELLFSAILEPNEAIKEHKGFLKILQD |
| SEQ ID NO: 50 | KGIKVIQLSDLVAETYTYHATQKEREAFIETWLDEAEPALTKDLRAKVRSYVLSKEGTP |
| | VAMVRTMMAGVSKQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KKIVNNKEAKFRRIVAINVPPMPNLMHLDTWLTMVDKDKFLYSPNMLSVLQVWEIDLSA |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM85 | SKINVYSEIGELKEVLVHIPGDEIRRISPSRLDELLFSAILEPNEAIKEHKGELKILQD |
| SEQ ID NO: 51 | KGIKVIQLSDLVAETYTYHATQKEREAFIETWLDEAEPALTKDLRAKVRSYVLSKEGTP |
| | VAMVRTMMAGVSKQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KKIVNNKEAKFKRIVAINVPPMPNLMHLDTWLIMVDKDKFLYSPNMLSVLQVWEIDLSA |
| | EIEMVETNKPLADVLESIIGVKPVLIPIAGKGATQLDIDIETHFDGINYLTIAPGVVVG |
| | YSRNIKTEAALRAAGVTVLSFEGNQLSLGMGSARCMSMPLVREDVK |
| McolM86 | SKINVYSEIGELKEVLVHTPGDEIRRISPSRLDELLFSAILEPNEAIKEHKGFLKILQD |
| SEQ ID NO: 52 | KGIKVIQLSDLVAETYTYHATQKEREAFIETWLDEAEPALTKDLRAKVRSYVLSKEGTP |
| | VAMVRTMMAGVSKQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KKIVNNKEAKFKKIVAINVPPMPNLMHLDTWLTMVDKDKFLYSPNMLSVLQVWEIDLSA |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM94 | SGINVYSEIGELKEVLVHTPGDEIRRISPSRLDELLFSAILEPNEAIKEHKGFLKILQD |
| SEQ ID NO: 53 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYATTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHFDGTNYLTIAPGVVVG |
| | YSRNIKTEAALRAAGVTVLSFEGNQLSLGMGSARCMSMPLVREDVA |
| McolM95 | SGINVYSEIGELKEVLVHTPGDEIRRISPSRLDELLFSAILEPNEAIKEHKGFLKILQD |
| SEQ ID NO: 54 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYRTTPHWFDRLDEGNIEGGDVFIYNNDILVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM96 | SGINVYSEIGELKEVLVHTPGDEIRRISPSRLDELLFSAILEPNEAIKEHKGFLKILQD |
| SEQ ID NO: 55 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYNTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM97 | SGINVYSEIGELKEVLVHTPGDEIRRISPSRLDELLFSAILEPNEAIKEHKGFLKILQD |
| SEQ ID NO: 56 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYDTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHFDGINYLTIAPGVVVG |
| | YSRNIKTEAALRAAGVTVLSFEGNQLSLGMGSARCMSMPLVREDVA |
| McolM98 | SGINVYSEIGELKEVLVHTPGDEIRRISPSRLDELLFSAILEPNEAIKEHKGFLKILQD |
| SEQ ID NO: 57 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYQTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM99 | SGINVYSEIGELKEVLVHTPGDEIRRISPSRLDELLFSAILEPNEAIKEHKGFLKILQD |
| SEQ ID NO: 58 | KGIKVIQLSDLVAETYTYHATQSEREAFIETWLDEAEPALTKDLRAKVRSYVLSKEGTP |
| | VAMVRIMMAGVSRQELNVESETELVVDPMPNLYFTRDPFASAGNGISLNNMKYVTRKRE |
| | TIFAEFIFATHPDYETTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHFDGINYLTIAPGVVVG |
| | YSRNIKTEAALRAAGVTVLSFEGNQLSLGMGSARCMSMPLVREDVA |
| McolM100 | SGINVYSEIGELKEVLVHTPGDEIRRISPSRLDELLFSAILEPNEAIKEHKGFLKILQD |
| SEQ ID NO: 59 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYGTTPHWFDRLDEGNIEGGDVFIYNNDTLVIGVSERTNKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHFDGINYLTIAPGVVVG |
| | YSRNIKTEAALRAAGVTVLSFEGNQLSLGMGSARCMSMPLVREDVA |
| McolM101 | SGINVYSEIGELKEVLVHTPGDEIRRISPSRLDELLFSAILEPNEAIKEHKGFLKILQD |
| SEQ ID NO: 60 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYHTTPHWFDRLDEGNIEGGDVFIYNNDTLVIGVSERTNKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHFDGTNYLTIAPGVVVG |
| | YSRNIKTEAALRAAGVTVLSFEGNQLSLGMGSARCMSMPLVREDVA |
| McolM102 | SGINVYSEIGELKEVLVHTPGDEIRRISPSRLDELLFSAILEPNEAIKEHKGFLKILQD |
| SEQ ID NO: 61 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYITTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVEINKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM103 | SGINVYSEIGELKEVLVHTPGDEIRRISPSRLDELLFSAILEPNEAIKEHKGFLKILQD |
| SEQ ID NO: 62 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYLTTPHWFDRLDEGNIEGGDVFIYNNDTLVIGVSERTNKEAILTIA |
| | KNIVNNKEAKFRRIVAINVPPMPNLMHLDTWLIMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM104 | SGINVYSEIGELKEVLVHTPGDEIRRISPSRLDELLFSAILEPNEAIKEHKGFLKILQD |
| SEQ ID NO: 63 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYMTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM105 | SGINVYSEIGELKEVLVHTPGDEIRRISPSRLDELLFSAILEPNEAIKEHKGFLKILQD |
| SEQ ID NO: 64 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYSTTPHWFDRLDEGNIEGGDVFIYNNDTLVIGVSERTNKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHFDGINYLTIAPGVVVG |
| | YSRNIKTEAALRAAGVTVLSFEGNQLSLGMGSARCMSMPLVREDVA |
| McolM106 | SGINVYSEIGELKEVLVHTPGDEIRRISPSRLDELLFSAILEPNEAIKEHKGFLKILQD |
| SEQ ID NO: 65 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYTTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM107 | SGINVYSEIGELKEVLVHTPGDEIRRISPSRLDELLFSAILEPNEAIKEHKGFLKILQD |
| SEQ ID NO: 66 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLAVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM108 | SGINVYSEIGELKEVLVHTPGDEIRRISPSRLDELLFSAILEPNEAIKEHKGFLKILQD |
| SEQ ID NO: 67 | KGIKVIQLSDLVAETYTYHATQSEREAFIETWLDEAEPALTKDLRAKVRSYVLSKEGTP |
| | VAMVRIMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERTNKEAILTIA |
| | KNIVNNKEAKFRRIVAINVPPMPNLMHLDTWLTMVDKDKFLYSPNMLSVLRVWEIDLSA |
| | EIEMVETNKPLADVLESIIGVRPVLIPIAGAGATQLDIDIETHFDGINYLTIAPGVVVG |
| | YSRNIKTEAALRAAGVTVLSFEGNQLSLGMGSARCMSMPLVREDVA |
| McolM109 | SGINVYSEIGELKEVLVHTPGDEIRRISPSRLDELLFSAILEPNEAIKEHKGFLKILQD |
| SEQ ID NO: 68 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERTNKEAILTIA |
| | KNIVNNKEAKFRRIVAINVPPMPNLMHLDTWLIMVDKDKFLYSPNMLSVLNVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM110 | SGINVYSEIGELKEVLVHTPGDEIRRISPSRLDELLFSAILEPNEAIKEHKGFLKILQD |
| SEQ ID NO: 69 | KGIKVIQLSDLVAETYTYHATQSEREAFIETWLDEAEPALIKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTIPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLDVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM111 | SGINVYSEIGELKEVLVHTPGDEIRRISPSRLDELLFSAILEPNEAIKEHKGFLKILQD |
| SEQ ID NO: 70 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLEVWEIDLSA |
| | EIEMVETNKPLADVLESIIGVRPVLIPIAGAGATQLDIDIETHFDGTNYLTIAPGVVVG |
| | YSRNIKTEAALRAAGVTVLSFEGNQLSLGMGSARCMSMPLVREDVA |
| McolM112 | SGINVYSEIGELKEVLVHTPGDEIRRISPSRLDELLFSAILEPNEAIKEHKGFLKILQD |
| SEQ ID NO: 71 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLGVWEIDLSA |
| | EIEMVETNKPLADVLESIIGVRPVLIPIAGAGATQLDIDIETHFDGTNYLTIAPGVVVG |
| | YSRNIKTEAALRAAGVTVLSFEGNQLSLGMGSARCMSMPLVREDVA |
| McolM113 | SGINVYSEIGELKEVLVHTPGDEIRRISPSRLDELLFSAILEPNEAIKEHKGFLKILQD |
| SEQ ID NO: 72 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERTNKEAILTIA |
| | KNIVNNKEAKFRRIVAINVPPMPNLMHLDTWLTMVDKDKFLYSPNMLSVLHVWEIDLSA |
| | EIEMVETNKPLADVLESIIGVRPVLIPIAGAGATQLDIDIETHFDGTNYLTIAPGVVVG |
| | YSRNIKTEAALRAAGVTVLSFEGNQLSLGMGSARCMSMPLVREDVA |
| McolM114 | SGINVYSEIGELKEVLVHTPGDEIRRISPSRLDELLFSAILEPNEAIKEHKGFLKILQD |
| SEQ ID NO: 73 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDILVIGVSERINKEAILTIA |
| | KNIVNNKEAKFRRIVAINVPPMPNLMHLDTWLTMVDKDKFLYSPNMLSVLIVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM115 | SGINVYSEIGELKEVLVHTPGDEIRRISPSRLDELLFSAILEPNEAIKEHKGFLKILQD |
| SEQ ID NO: 74 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDILVIGVSERINKEAILTIA |
| | KNIVNNKEAKFRRIVAINVPPMPNLMHLDTWLTMVDKDKFLYSPNMLSVLLVWEIDLSA |
| | EIEMVETNKPLADVLESIIGVRPVLIPIAGAGATQLDIDIETHFDGINYLTIAPGVVVG |
| | YSRNIKTEAALRAAGVTVLSFEGNQLSLGMGSARCMSMPLVREDVA |
| McolM116 | SGINVYSEIGELKEVLVHTPGDEIRRISPSRLDELLFSAILEPNEAIKEHKGFLKILQD |
| SEQ ID NO: 75 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLMVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM117 | SGINVYSEIGELKEVLVHTPGDEIRRISPSRLDELLFSAILEPNEAIKEHKGFLKILQD |
| SEQ ID NO: 76 | KGIKVIQLSDLVAETYTYHATQSEREAFIETWLDEAEPALIKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDILVIGVSERINKEAILTIA |
| | KNIVNNKEAKFRRIVAINVPPMPNLMHLDTWLTMVDKDKFLYSPNMLSVLSVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM118 | SGINVYSEIGELKEVLVHIPGDEIRRISPSRLDELLFSAILEPNEAIKEHKGFLKILQD |
| SEQ ID NO: 77 | KGIKVIQLSDLVAETYTYHATQSEREAFIETWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLIVWEIDLSA |
| | EIEMVETNKPLADVLESIIGVRPVLIPIAGAGATQLDIDIETHEDGINYLTIAPGVVVG |
| | YSRNIKTEAALRAAGVTVLSFEGNQLSLGMGSARCMSMPLVREDVA |
| McolM119 | SGINVYSEIGELKEVLVHTPGDEIRRISPSRLDELLFSAILEPNEAIKEHKGFLKILQD |
| SEQ ID NO: 78 | KGIKVIQLSDLVAETYTYHATQSEREAFIETWLDEAEPALTKDLRAKVRSYVLSKEGTP |
| | VAMVRIMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERTNKEAILTIA |
| | KNIVNNKEAKFRRIVAINVPPMPNLMHLDTWLTMVDKDKFLYSPNMLSVLVVWEIDLSA |
| | EIEMVETNKPLADVLESIIGVRPVLIPIAGAGATQLDIDIETHFDGTNYLTIAPGVVVG |
| | YSRNIKTEAALRAAGVTVLSFEGNQLSLGMGSARCMSMPLVREDVA |
| McolM120 | SGINVYSEIGELKEVLVHTPGDEIRRISPSRLDELLFSAILEPNEAIKEHKGFLKILQD |
| SEQ ID NO: 79 | KGIKVIQLSDLVAETYTYHATQSEREAFIEAWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM121 | SGINVYSEIGELKEVLVHTPGDEIRRISPSRLDELLFSAILEPNEAIKEHKGFLKILQD |
| SEQ ID NO: 80 | KGIKVIQLSDLVAETYTYHATQSEREAFIERWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM122 | SGINVYSEIGELKEVLVHTPGDEIRRISPSRLDELLFSAILEPNEAIKEHKGELKILQD |
| SEQ ID NO: 81 | KGIKVIQLSDLVAETYTYHATQSEREAFIENWLDEAEPALTKDLRAKVRSYVLSKEGTP |
| | VAMVRIMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHFDGTNYLTIAPGVVVG |
| | YSRNIKTEAALRAAGVTVLSFEGNQLSLGMGSARCMSMPLVREDVA |
| McolM123 | SGINVYSEIGELKEVLVHTPGDEIRRISPSRLDELLFSAILEPNEAIKEHKGFLKILQD |
| SEQ ID NO: 82 | KGIKVIQLSDLVAETYTYHATQSEREAFIEDWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERTNKEAILTIA |
| | KNIVNNKEAKFRRIVAINVPPMPNLMHLDTWLIMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM124 | SGINVYSEIGELKEVLVHTPGDEIRRISPSRLDELLFSAILEPNEAIKEHKGFLKILQD |
| SEQ ID NO: 83 | KGIKVIQLSDLVAETYTYHATQSEREAFIEQWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVITPHWFDRLDEGNIEGGDVFIYNNDILVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM125 | SGINVYSEIGELKEVLVHTPGDEIRRISPSRLDELLFSAILEPNEAIKEHKGFLKILQD |
| SEQ ID NO: 84 | KGIKVIQLSDLVAETYTYHATQSEREAFIEEWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVITPHWFDRLDEGNIEGGDVFIYNNDILVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM126 | SGINVYSEIGELKEVLVHTPGDEIRRISPSRLDELLFSAILEPNEAIKEHKGFLKILQD |
| SEQ ID NO: 85 | KGIKVIQLSDLVAETYTYHATQSEREAFIEGWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLIMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM127 | SGINVYSEIGELKEVLVHIPGDEIRRISPSRLDELLFSAILEPNEAIKEHKGFLKILQD |
| SEQ ID NO: 86 | KGIKVIQLSDLVAETYTYHATQSEREAFIEHWLDEAEPALTKDLRAKVRSYVLSKEGTP |
| | VAMVRIMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERTNKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM128 | SGINVYSEIGELKEVLVHIPGDEIRRISPSRLDELLFSAILEPNEAIKEHKGFLKILQD |
| SEQ ID NO: 87 | KGIKVIQLSDLVAETYTYHATQSEREAFIEIWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLIMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM129 | SGINVYSEIGELKEVLVHTPGDEIRRISPSRLDELLFSAILEPNEAIKEHKGFLKILQD |
| SEQ ID NO: 88 | KGIKVIQLSDLVAETYTYHATQSEREAFIELWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLIMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM130 | SGINVYSEIGELKEVLVHTPGDEIRRISPSRLDELLFSAILEPNEAIKEHKGFLKILQD |
| SEQ ID NO: 89 | KGIKVIQLSDLVAETYTYHATQSEREAFIEMWLDEAEPALIKDLRAKVRSYVLSKEGTP |
| | VAMVRIMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM131 | SGINVYSEIGELKEVLVHTPGDEIRRISPSRLDELLFSAILEPNEAIKEHKGFLKILQD |
| SEQ ID NO: 90 | KGIKVIQLSDLVAETYTYHATQSEREAFIESWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDILVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM132 | SGINVYSEIGELKEVLVHTPGDEIRRISPSRLDELLFSAILEPNEAIKEHKGFLKILQD |
| SEQ ID NO: 91 | KGIKVIQLSDLVAETYTYHATQSEREAFIEVWLDEAEPALTKDLRAKVRSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM133 | SGINVYSEIGELKEVLVHTPGDEIRRISPSRLDELLFSAILEPNEAIKEHKGFLKILQD |
| SEQ ID NO: 92 | KGIKVIQLSDLVAETYTYHATQSEREAFIETWLDEAEPALTKDLRAKVASYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGINYLTIAPGVVVG |
| | YSRNIKTEAALRAAGVTVLSFEGNQLSLGMGSARCMSMPLVREDVA |
| McolM134 | SGINVYSEIGELKEVLVHTPGDEIRRISPSRLDELLFSAILEPNEAIKEHKGFLKILQD |
| SEQ ID NO: 93 | KGIKVIQLSDLVAETYTYHATQSEREAFIETWLDEAEPALTKDLRAKVNSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVEINKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM135 | SGINVYSEIGELKEVLVHTPGDEIRRISPSRLDELLFSAILEPNEAIKEHKGFLKILQD |
| SEQ ID NO: 94 | KGIKVIQLSDLVAETYTYHATQSEREAFIETWLDEAEPALTKDLRAKVDSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM136 | SGINVYSEIGELKEVLVHTPGDEIRRISPSRLDELLFSAILEPNEAIKEHKGFLKILQD |
| SEQ ID NO: 95 | KGIKVIQLSDLVAETYTYHATQSEREAFIETWLDEAEPALTKDLRAKVQSYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHFDGTNYLTIAPGVVVG |
| | YSRNIKTEAALRAAGVTVLSFEGNQLSLGMGSARCMSMPLVREDVA |
| McolM137 | SGINVYSEIGELKEVLVHTPGDEIRRISPSRLDELLFSAILEPNEAIKEHKGFLKILQD |
| SEQ ID NO: 96 | KGIKVIQLSDLVAETYTYHATQSEREAFIETWLDEAEPALTKDLRAKVESYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM138 | SGINVYSEIGELKEVLVHTPGDEIRRISPSRLDELLFSAILEPNEAIKEHKGFLKILQD |
| SEQ ID NO: 97 | KGIKVIQLSDLVAETYTYHATQSEREAFIETWLDEAEPALTKDLRAKVGSYVLSKEGTP |
| | VAMVRTMMAGVSRQELNVESETELVVDPMPNLYFTRDPFASAGNGISLNNMKYVTRKRE |
| | TIFAEFIFATHPDYVTIPHWFDRLDEGNIEGGDVFIYNNDILVIGVSERINKEAILTIA |
| | KNIVNNKEAKFRRIVAINVPPMPNLMHLDTWLIMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM139 | SGINVYSEIGELKEVLVHTPGDEIRRISPSRLDELLFSAILEPNEAIKEHKGFLKILQD |
| SEQ ID NO: 98 | KGIKVIQLSDLVAETYTYHATQSEREAFIETWLDEAEPALTKDLRAKVHSYVLSKEGTP |
| | VAMVRIMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM140 | SGINVYSEIGELKEVLVHTPGDEIRRISPSRLDELLFSAILEPNEAIKEHKGFLKILQD |
| SEQ ID NO: 99 | KGIKVIQLSDLVAETYTYHATQSEREAFIETWLDEAEPALTKDLRAKVISYVLSKEGTP |
| | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLIMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM141 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVLSYVLSKEGTP |
| 100 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM142 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVMSYVLSKEGTP |
| 101 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERTNKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM143 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVSSYVLSKEGTP |
| 102 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDILVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM144 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVTSYVLSKEGTP |
| 103 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLIMVDKDKFLYSPNMLSVLQVWEIDLSA |
| | EIEMVETNKPLADVLESIIGVRPVLIPIAGAGATQLDIDIETHFDGINYLTIAPGVVVG |
| | YSRNIKTEAALRAAGVTVLSFEGNQLSLGMGSARCMSMPLVREDVA |
| McolM145 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVVSYVLSKEGTP |
| 104 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM146 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 105 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNADTLVIGVSERINKEAILTIA |
| | KNIVNNKEAKFRRIVAINVPPMPNLMHLDTWLIMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM147 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 106 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNRDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM148 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 107 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNDDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM149 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 108 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNQDTLVIGVSERINKEAILTIA |
| | KNIVNNKEAKFRRIVAINVPPMPNLMHLDTWLIMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM150 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 109 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNEDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM151 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 110 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNGDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHFDGINYLTIAPGVVVG |
| | YSRNIKTEAALRAAGVTVLSFEGNQLSLGMGSARCMSMPLVREDVA |
| McolM152 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 111 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNHDTLVIGVSERINKEAILTIA |
| | KNIVNNKEAKFRRIVAINVPPMPNLMHLDTWLIMVDKDKFLYSPNMLSVLQVWEIDLSA |
| | EIEMVETNKPLADVLESIIGVRPVLIPIAGAGATQLDIDIETHFDGTNYLTIAPGVVVG |
| | YSRNIKTEAALRAAGVTVLSFEGNQLSLGMGSARCMSMPLVREDVA |
| McolM153 | SGINVYSEIGELKEVLVHIPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 112 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNIDILVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGINYLTIAPGVVVG |
| | YSRNIKTEAALRAAGVTVLSFEGNQLSLGMGSARCMSMPLVREDVA |
| McolM154 | SGINVYSEIGELKEVLVHIPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 113 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNLDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM155 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 114 | VAMVRIMMAGVSRQELNVESETELVVDPMPNLYFTRDPFASAGNGISLNNMKYVTRKRE |
| | TIFAEFIFATHPDYVTTPHWFDRLDEGNIEGGDVFIYNMDTLVIGVSERINKEAILTIA |
| | KNIVNNKEAKFRRIVAINVPPMPNLMHLDTWLIMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM156 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 115 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNSDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHFDGTNYLTIAPGVVVG |
| | YSRNIKTEAALRAAGVTVLSFEGNQLSLGMGSARCMSMPLVREDVA |
| McolM157 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALIKDLRAKVRSYVLSKEGTP |
| 116 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNTDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHFDGINYLTIAPGVVVG |
| | YSRNIKTEAALRAAGVTVLSFEGNQLSLGMGSARCMSMPLVREDVA |
| McolM158 | SGINVYSEIGELKEVLVHIPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 117 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNVDTLVIGVSERINKEAILTIA |
| | KNIVNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM159 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 118 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIANNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM160 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 119 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIRNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM161 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 120 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNINNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM162 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 121 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERTNKEAILTIA |
| | KNIDNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHFDGINYLTIAPGVVVG |
| | YSRNIKTEAALRAAGVTVLSFEGNQLSLGMGSARCMSMPLVREDVA |
| McolM163 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 122 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERTNKEAILTIA |
| | KNIQNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM164 | SGINVYSEIGELKEVLVHIPGDEIRRISPSRLDELLFSAILEPNEAIKEHKGFLKILQD |
| SEQ ID NO: | KGIKVIQLSDLVAETYTYHATQSEREAFIETWLDEAEPALIKDLRAKVRSYVLSKEGTP |
| 123 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERTNKEAILTIA |
| | KNIENNKEAKFRRIVAINVPPMPNLMHLDTWLIMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM165 | SGINVYSEIGELKEVLVHIPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 124 | VAMVRIMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIGNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHFDGTNYLTIAPGVVVG |
| | YSRNIKTEAALRAAGVTVLSFEGNQLSLGMGSARCMSMPLVREDVA |
| McolM166 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 125 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERTNKEAILTIA |
| | KNIHNNKEAKFRRIVAINVPPMPNLMHLDTWLIMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM167 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 126 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIINNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM168 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 127 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNILNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM169 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 128 | VAMVRTMMAGVSRQELNVESETELVVDPMPNLYFTRDPFASAGNGISLNNMKYVTRKRE |
| | TIFAEFIFATHPDYVTTPHWEDRLDEGNIEGGDVFIYNNDTLVIGVSERTNKEAILTIA |
| | KNIMNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM170 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALIKDLRAKVRSYVLSKEGTP |
| 129 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNISNNKEAKFRRIVAINVPPMPNLMHLDTWLTMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM171 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALIKDLRAKVRSYVLSKEGTP |
| 130 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNITNNKEAKFRRIVAINVPPMPNLMHLDTWLIMVDKDKFLYSPNMLSVLQVWEIDLSA |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM172 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 131 | VAMVRIMMAGVSRQELNVESETELVVDPMPNLYFTRDPFASAGNGISLNNMKYVTRKRE |
| | TIFAEFIFATHPDYVTIPHWFDRLDEGNIEGGDVFIYNNDILVIGVSERINKEAILTIA |
| | KNIVNNKEAKFRRIVAINVPPMPNLMHLDTWLTMVDKDKFLYSPNMLSVLQVWEIDLSR |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM173 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 132 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDILVIGVSERINKEAILTIA |
| | KNIVNNKEAKFRRIVAINVPPMPNLMHLDTWLTMVDKDKFLYSPNMLSVLQVWEIDLSN |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM174 | SGINVYSEIGELKEVLVHIPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 133 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLIMVDKDKFLYSPNMLSVLQVWEIDLSD |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM175 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 134 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSQ |
| | EIEMVETNKPLADVLESIIGVRPVLIPIAGAGATQLDIDIETHEDGINYLTIAPGVVVG |
| | YSRNIKTEAALRAAGVTVLSFEGNQLSLGMGSARCMSMPLVREDVA |
| McolM176 | SGINVYSEIGELKEVLVHIPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 135 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERTNKEAILTIA |
| | KNIVNNKEAKFRRIVAINVPPMPNLMHLDTWLTMVDKDKFLYSPNMLSVLQVWEIDLSE |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM177 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALIKDLRAKVRSYVLSKEGTP |
| 136 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERINKEAILTIA |
| | KNIVNNKEAKFRRIVAINVPPMPNLMHLDTWLTMVDKDKFLYSPNMLSVLQVWEIDLSG |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM178 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 137 | VAMVRTMMAGVSRQELNVESETELVVDPMPNLYFTRDPFASAGNGISLNNMKYVTRKRE |
| | TIFAEFIFATHPDYVITPHWFDRLDEGNIEGGDVFIYNNDILVIGVSERINKEAILTIA |
| | KNIVNNKEAKFRRIVAINVPPMPNLMHLDTWLTMVDKDKFLYSPNMLSVLQVWEIDLSH |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM179 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALIKDLRAKVRSYVLSKEGIP |
| 138 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDILVIGVSERINKEAILTIA |
| | KNIVNNKEAKFRRIVAINVPPMPNLMHLDTWLTMVDKDKFLYSPNMLSVLQVWEIDLSI |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM180 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 139 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDILVIGVSERINKEAILTIA |
| | KNIVNNKEAKFRRIVAINVPPMPNLMHLDTWLTMVDKDKFLYSPNMLSVLQVWEIDLSL |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM181 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 140 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDILVIGVSERINKEAILTIA |
| | KNIVNNKEAKFRRIVAINVPPMPNLMHLDTWLTMVDKDKFLYSPNMLSVLQVWEIDLSM |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM182 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 141 | VAMVRTMMAGVSRQELNVESETELVVDPMPNLYFTRDPFASAGNGISLNNMKYVTRKRE |
| | TIFAEFIFATHPDYVTTPHWFDRLDEGNIEGGDVFIYNNDTLVIGVSERTNKEAILTIA |
| | KNIVNNKEAKFRRIVAINVPPMPNLMHLDTWLTMVDKDKFLYSPNMLSVLQVWEIDLSS |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM183 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 142 | VAMVRTMMAGVSRQELNVESETELVVDPMPNLYFTRDPFASAGNGISLNNMKYVTRKRE |
| | TIFAEFIFATHPDYVTTPHWEDRLDEGNIEGGDVFIYNNDILVIGVSERINKEAILTIA |
| | KNIVNNKEAKFRRIVAINVPPMPNLMHLDTWLTMVDKDKFLYSPNMLSVLQVWEIDLST |
| | EIEMVETNKPLADVLESIIGVRPVLIPIAGAGATQLDIDIETHFDGTNYLTIAPGVVVG |
| | YSRNIKTEAALRAAGVTVLSFEGNQLSLGMGSARCMSMPLVREDVA |
| McolM184 | SGINVYSEIGELKEVLVHTPGDEIRRISPSRLDELLFSAILEPNEAIKEHKGFLKILQD |
| SEQ ID NO: | KGIKVIQLSDLVAETYTYHATQSEREAFIETWLDEAEPALTKDLRAKVRSYVLSKEGTP |
| 143 | VAMVRTMMAGVSRQELNVESETELVVDPMPNLYFTRDPFASAGNGISLNNMKYVTRKRE |
| | TIFAEFIFATHPDYVTTPHWEDRLDEGNIEGGDVFIYNNDILVIGVSERINKEAILTIA |
| | KNIVNNKEAKFRRIVAINVPPMPNLMHLDTWLTMVDKDKFLYSPNMLSVLQVWEIDLSV |
| | EIEMVETNKPLADVLESIIGVRPVLIPIAGAGATQLDIDIETHEDGTNYLTIAPGVVVG |
| | YSRNIKTEAALRAAGVTVLSFEGNQLSLGMGSARCMSMPLVREDVA |
| McolM188 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVASYVLSKEGTP |
| 144 | VAMVRTMMAGVSKQELNVESETELVVDPMPNLYFTRDPFASAGNGISLNNMKYVTRKRE |
| | TIFAEFIFATHPDYGITPHWFDRLDEGNIEGGDVFIYNVDTLVIGVSERINKEAILTIA |
| | KKIDNNKEAEFKKIVAINVPPMPNLMHLDTWLTMVDKDKFLYSPNMLSVLAVWEIDLSI |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM189 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEKWLDEAEPALIKDLRAKVASYVLSKEGTP |
| 145 | VAMVRTMMAGVSKQELNVESETELVVDPMPNLYFTRDPFASAGNGISLNNMKYVTRKRE |
| | TIFAEFIFATHPDYGTTPHWFDRLDEGNIEGGDVFIYNVDTLVIGVSERINKEAILTIA |
| | KKIDNNKEAEFKKIVAINVPPMPNLMHLDTWLTMVDKDKFLYSPNMLSVLAVWEIDLSI |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM190 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALIKDLRAKVKSYVLSKEGTP |
| 146 | VAMVRTMMAGVSKQELNVESETELVVDPMPNLYFTRDPFASAGNGISLNNMKYVTRKRE |
| | TIFAEFIFATHPDYGTTPHWFDRLDEGNIEGGDVFIYNVDTLVIGVSERINKEAILTIA |
| | KKIDNNKEAEFKKIVAINVPPMPNLMHLDTWLIMVDKDKFLYSPNMLSVLAVWEIDLSI |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM191 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVASYVLSKEGTP |
| 147 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNVDTLVIGVSERINKEAILTIA |
| | KKIDNNKEAEFKKIVAINVPPMPNLMHLDTWLTMVDKDKFLYSPNMLSVLAVWEIDLSI |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM192 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVASYVLSKEGTP |
| 148 | VAMVRTMMAGVSKQELNVESETELVVDPMPNLYFTRDPFASAGNGISLNNMKYVTRKRE |
| | TIFAEFIFATHPDYGTTPHWFDRLDEGNIEGGDVFIYNKDTLVIGVSERINKEAILTIA |
| | KKIDNNKEAEFKKIVAINVPPMPNLMHLDTWLTMVDKDKFLYSPNMLSVLAVWEIDLSI |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM193 | SKINVYSEIGELKEVLVHIPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVASYVLSKEGTP |
| 149 | VAMVRTMMAGVSKQELNVESETELVVDPMPNLYFTRDPFASAGNGISLNNMKYVTRKRE |
| | TIFAEFIFATHPDYGTTPHWFDRLDEGNIEGGDVFIYNVDTLVIGVSERINKEAILTIA |
| | KKIKNNKEAEFKKIVAINVPPMPNLMHLDTWLIMVDKDKFLYSPNMLSVLAVWEIDLSI |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM194 | SKINVYSEIGELKEVLVHIPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVASYVLSKEGTP |
| 150 | VAMVRTMMAGVSKQELNVESETELVVDPMPNLYFTRDPFASAGNGISLNNMKYVTRKRE |
| | TIFAEFIFATHPDYGITPHWFDRLDEGNIEGGDVFIYNVDILVIGVSERINKEAILTIA |
| | KKIDNNKEAEFKKIVAINVPPMPNLMHLDTWLTMVDKDKFLYSPNMLSVLKVWEIDLSI |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM195 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVASYVLSKEGTP |
| 151 | VAMVRTMMAGVSKQELNVESETELVVDPMPNLYFTRDPFASAGNGISLNNMKYVTRKRE |
| | TIFAEFIFATHPDYGITPHWFDRLDEGNIEGGDVFIYNVDTLVIGVSERINKEAILTIA |
| | KKIDNNKEAEFKKIVAINVPPMPNLMHLDTWLTMVDKDKFLYSPNMLSVLAVWEIDLSK |
| | EIEMVETNKPLADVLESIIGVKPVLIPIAGKGATQLDIDIETHFDGTNYLTIAPGVVVG |
| | YSRNIKTEAALRAAGVTVLSFEGNQLSLGMGSARCMSMPLVREDVK |
| McolM196 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEKWLDEAEPALTKDLRAKVKSYVLSKEGTP |
| 152 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNKDTLVIGVSERINKEAILTIA |
| | KKIKNNKEAEFKKIVAINVPPMPNLMHLDTWLIMVDKDKFLYSPNMLSVLKVWEIDLSK |
| | EIEMVETNKPLADVLESIIGVKPVLIPIAGKGATQLDIDIETHFDGINYLTIAPGVVVG |
| | YSRNIKTEAALRAAGVTVLSFEGNQLSLGMGSARCMSMPLVREDVK |
| McolM197 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVKSYVLSKEGTP |
| 153 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNKDTLVIGVSERTNKEAILTIA |
| | KKIKNNKEAEFKKIVAINVPPMPNLMHLDTWLIMVDKDKFLYSPNMLSVLKVWEIDLSK |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM198 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEKWLDEAEPALTKDLRAKVASYVLSKEGTP |
| 154 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNKDTLVIGVSERTNKEAILTIA |
| | KKIKNNKEAEFKKIVAINVPPMPNLMHLDTWLTMVDKDKFLYSPNMLSVLKVWEIDLSK |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM199 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEKWLDEAEPALTKDLRAKVKSYVLSKEGTP |
| 155 | VAMVRIMMAGVSKQELNVESETELVVDPMPNLYFTRDPFASAGNGISLNNMKYVTRKRE |
| | TIFAEFIFATHPDYGTTPHWFDRLDEGNIEGGDVFIYNKDTLVIGVSERTNKEAILTIA |
| | KKIKNNKEAEFKKIVAINVPPMPNLMHLDTWLTMVDKDKFLYSPNMLSVLKVWEIDLSK |
| | EIEMVETNKPLADVLESIIGVKPVLIPIAGKGATQLDIDIETHFDGTNYLTIAPGVVVG |
| | YSRNIKTEAALRAAGVTVLSFEGNQLSLGMGSARCMSMPLVREDVK |
| McolM200 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEKWLDEAEPALTKDLRAKVKSYVLSKEGTP |
| 156 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNVDTLVIGVSERINKEAILTIA |
| | KKIKNNKEAEFKKIVAINVPPMPNLMHLDTWLTMVDKDKFLYSPNMLSVLKVWEIDLSK |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM201 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEKWLDEAEPALTKDLRAKVKSYVLSKEGTP |
| 157 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNKDTLVIGVSERINKEAILTIA |
| | KKIDNNKEAEFKKIVAINVPPMPNLMHLDTWLIMVDKDKFLYSPNMLSVLKVWEIDLSK |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM202 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEKWLDEAEPALIKDLRAKVKSYVLSKEGTP |
| 158 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNKDTLVIGVSERINKEAILTIA |
| | KKIKNNKEAEFKKIVAINVPPMPNLMHLDTWLTMVDKDKFLYSPNMLSVLAVWEIDLSK |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM203 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEKWLDEAEPALTKDLRAKVKSYVLSKEGTP |
| 159 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNKDTLVIGVSERINKEAILTIA |
| | KKIKNNKEAEFKKIVAINVPPMPNLMHLDTWLTMVDKDKFLYSPNMLSVLKVWEIDLSI |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM204 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALIKDLRAKVASYVLSKEGTP |
| 160 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNKDTLVIGVSERINKEAILTIA |
| | KKIKNNKEAEFKKIVAINVPPMPNLMHLDTWLIMVDKDKFLYSPNMLSVLKVWEIDLSK |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM205 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVKSYVLSKEGTP |
| 161 | VAMVRTMMAGVSKQELNVESETELVVDPMPNLYFTRDPFASAGNGISLNNMKYVTRKRE |
| | TIFAEFIFATHPDYGITPHWFDRLDEGNIEGGDVFIYNKDTLVIGVSERINKEAILTIA |
| | KKIKNNKEAEFKKIVAINVPPMPNLMHLDTWLTMVDKDKFLYSPNMLSVLKVWEIDLSK |
| | EIEMVETNKPLADVLESIIGVKPVLIPIAGKGATQLDIDIETHFDGINYLTIAPGVVVG |
| | YSRNIKTEAALRAAGVTVLSFEGNQLSLGMGSARCMSMPLVREDVK |
| McolM206 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALIKDLRAKVKSYVLSKEGTP |
| 162 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNVDTLVIGVSERINKEAILTIA |
| | KKIKNNKEAEFKKIVAINVPPMPNLMHLDTWLTMVDKDKFLYSPNMLSVLKVWEIDLSK |
| | EIEMVEINKPLADVLESIIGVKPVLIPIAGKGATQLDIDIETHFDGINYLTIAPGVVVG |
| | YSRNIKTEAALRAAGVTVLSFEGNQLSLGMGSARCMSMPLVREDVK |
| McolM207 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVKSYVLSKEGTP |
| 163 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNKDTLVIGVSERINKEAILTIA |
| | KKIDNNKEAEFKKIVAINVPPMPNLMHLDTWLTMVDKDKFLYSPNMLSVLKVWEIDLSK |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM208 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVKSYVLSKEGTP |
| 164 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNKDTLVIGVSERTNKEAILTIA |
| | KKIKNNKEAEFKKIVAINVPPMPNLMHLDTWLIMVDKDKFLYSPNMLSVLAVWEIDLSK |
| | EIEMVETNKPLADVLESIIGVKPVLIPIAGKGATQLDIDIETHFDGINYLTIAPGVVVG |
| | YSRNIKTEAALRAAGVTVLSFEGNQLSLGMGSARCMSMPLVREDVK |
| McolM209 | SKINVYSEIGELKEVLVHIPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVKSYVLSKEGTP |
| 165 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNKDTLVIGVSERINKEAILTIA |
| | KKIKNNKEAEFKKIVAINVPPMPNLMHLDTWLIMVDKDKFLYSPNMLSVLKVWEIDLSI |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM210 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVASYVLSKEGTP |
| 166 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNKDTLVIGVSERINKEAILTIA |
| | KKIKNNKEAEFKKIVAINVPPMPNLMHLDTWLTMVDKDKFLYSPNMLSVLKVWEIDLSI |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM211 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVKSYVLSKEGTP |
| 167 | VAMVRTMMAGVSKQELNVESETELVVDPMPNLYFTRDPFASAGNGISLNNMKYVTRKRE |
| | TIFAEFIFATHPDYGTTPHWFDRLDEGNIEGGDVFIYNKDTLVIGVSERINKEAILTIA |
| | KKIKNNKEAEFKKIVAINVPPMPNLMHLDTWLTMVDKDKFLYSPNMLSVLKVWEIDLSI |
| | EIEMVETNKPLADVLESIIGVKPVLIPIAGKGATQLDIDIETHFDGINYLTIAPGVVVG |
| | YSRNIKTEAALRAAGVTVLSFEGNQLSLGMGSARCMSMPLVREDVK |
| McolM212 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVKSYVLSKEGTP |
| 168 | VAMVRTMMAGVSKQELNVESETELVVDPMPNLYFTRDPFASAGNGISLNNMKYVTRKRE |
| | TIFAEFIFATHPDYKITPHWEDRLDEGNIEGGDVFIYNVDILVIGVSERINKEAILTIA |
| | KKIKNNKEAEFKKIVAINVPPMPNLMHLDTWLTMVDKDKFLYSPNMLSVLKVWEIDLSI |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM213 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVKSYVLSKEGTP |
| 169 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNKDTLVIGVSERINKEAILTIA |
| | KKIDNNKEAEFKKIVAINVPPMPNLMHLDTWLTMVDKDKFLYSPNMLSVLKVWEIDLSI |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM214 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVKSYVLSKEGTP |
| 170 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNKDTLVIGVSERINKEAILTIA |
| | KKIKNNKEAEFKKIVAINVPPMPNLMHLDTWLTMVDKDKFLYSPNMLSVLAVWEIDLSI |
| | EIEMVETNKPLADVLESIIGVKPVLIPIAGKGATQLDIDIETHFDGTNYLTIAPGVVVG |
| | YSRNIKTEAALRAAGVTVLSFEGNQLSLGMGSARCMSMPLVREDVK |
| McolM215 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVASYVLSKEGTP |
| 171 | VAMVRTMMAGVSKQELNVESETELVVDPMPNLYFTRDPFASAGNGISLNNMKYVTRKRE |
| | TIFAEFIFATHPDYGTTPHWFDRLDEGNIEGGDVFIYNKDTLVIGVSERINKEAILTIA |
| | KKIKNNKEAEFKKIVAINVPPMPNLMHLDTWLTMVDKDKFLYSPNMLSVLKVWEIDLSI |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM216 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVASYVLSKEGTP |
| 172 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNVDILVIGVSERINKEAILTIA |
| | KKIKNNKEAEFKKIVAINVPPMPNLMHLDTWLTMVDKDKFLYSPNMLSVLKVWEIDLSI |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM217 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVASYVLSKEGTP |
| 173 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNKDTLVIGVSERINKEAILTIA |
| | KKIDNNKEAEFKKIVAINVPPMPNLMHLDTWLTMVDKDKFLYSPNMLSVLKVWEIDLSI |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM218 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVASYVLSKEGTP |
| 174 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNKDTLVIGVSERINKEAILTIA |
| | KKIKNNKEAEFKKIVAINVPPMPNLMHLDTWLTMVDKDKFLYSPNMLSVLAVWEIDLSI |
| | EIEMVETNKPLADVLESIIGVKPVLIPIAGKGATQLDIDIETHFDGINYLTIAPGVVVG |
| | YSRNIKTEAALRAAGVTVLSFEGNQLSLGMGSARCMSMPLVREDVK |
| McolM219 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALIKDLRAKVASYVLSKEGTP |
| 175 | VAMVRTMMAGVSKQELNVESETELVVDPMPNLYFTRDPFASAGNGISLNNMKYVTRKRE |
| | TIFAEFIFATHPDYGITPHWFDRLDEGNIEGGDVFIYNKDTLVIGVSERINKEAILTIA |
| | KKIKNNKEAEFKKIVAINVPPMPNLMHLDTWLTMVDKDKFLYSPNMLSVLAVWEIDLSI |
| | EIEMVEINKPLADVLESIIGVKPVLIPIAGKGATQLDIDIETHEDGINYLTIAPGVVVG |
| | YSRNIKTEAALRAAGVTVLSFEGNQLSLGMGSARCMSMPLVREDVK |
| McolM220 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALTKDLRAKVASYVLSKEGTP |
| 176 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNVDTLVIGVSERINKEAILTIA |
| | KKIKNNKEAEFKKIVAINVPPMPNLMHLDTWLTMVDKDKFLYSPNMLSVLAVWEIDLSI |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |
| McolM221 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEVWLDEAEPALIKDLRAKVASYVLSKEGTP |
| 177 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNKDTLVIGVSERINKEAILTIA |
| | KKIDNNKEAEFKKIVAINVPPMPNLMHLDTWLTMVDKDKFLYSPNMLSVLAVWEIDLSI |
| | EIEMVETNKPLADVLESIIGVKPVLIPIAGKGATQLDIDIETHFDGTNYLTIAPGVVVG |
| | YSRNIKTEAALRAAGVTVLSFEGNQLSLGMGSARCMSMPLVREDVK |
| McolM222 | SKINVYSEIGELKEVLVHTPGDEIRRISPSRLDELLFSAILEPNEAIKEHKGFLKILQD |
| SEQ ID NO: | KGIKVIQLSDLVAETYTYHATQKEREAFIEKWLDEAEPALTKDLRAKVASYVLSKEGTP |
| 178 | VAMVRTMMAGVSKQELNVESETELVVDPMPNLYFTRDPFASAGNGISLNNMKYVTRKRE |
| | TIFAEFIFATHPDYKTTPHWFDRLDEGNIEGGDVFIYNVDTLVIGVSERINKEAILTIA |
| | KKIKNNKEAEFKKIVAINVPPMPNLMHLDTWLTMVDKDKFLYSPNMLSVLKVWEIDLSI |
| | EIEMVETNKPLADVLESIIGVKPVLIPIAGKGATQLDIDIETHEDGTNYLTIAPGVVVG |
| | YSRNIKTEAALRAAGVTVLSFEGNQLSLGMGSARCMSMPLVREDVK |

Thus, in some embodiments, an ADI protein comprises, consists, or consists essentially of an amino acid sequence selected from Table A1.1 and A1.2 (e.g., SEQ ID NOs: 2-178), excluding SEQ ID NO:1. Also included are variants and/or fragments thereof having ADI activity. For example, in certain embodiments, an ADI protein comprises, consists, or consists essentially of an amino acid sequence that is at least 90, 95, 96, 97, 98, 99, or 100% identical to a reference amino sequence selected from Table A1.1 and A1.2 (e.g., SEQ ID NOs: 2-178), excluding SEQ ID NO:1.

A “variant” sequence refers to a polypeptide or polynucleotide sequence that differs from a reference sequence by one or more substitutions, deletions (e.g., truncations), additions, and/or insertions. Certain variants thus include fragments of a reference sequence described herein. Variant polypeptides are biologically active, that is, they continue to possess the enzymatic or binding activity of a reference polypeptide. Such variants may result from, for example, genetic polymorphism and/or from human manipulation.

In certain embodiments, a variant and/or fragment of an ADI from Table A1.1 or Table A1.2 retains one or more of the lysine substitutions selected from K2G, K13E, K63N, K82S, K90T, K90V, K101D, K106L, K108R, K108A, K131R, K170R, K175R, K192V, K192C, K216N, K216V, K229L, K237N, K238N, K24.0V, K243T, K246E, K248R, K249R, K273R, K275A, K287Q, K287C, K295A, K304L, K317R, K326A, and K400A (relative to SEQ ID NO: 1). In some embodiments, a variant and/or fragment of an ADI from Table A1 retains 1, 2, 3, 4, 5, 6, 7, 8, 9, 10, 11, 12, 13, 14, 15, 16, 17, 18, 19, 20, 21, 22, 23, 24, 25, 26, 27, 28, or 29 of the lysine substitutions selected from K2G, K13E, K63N, K82S, K90T, K90V, K101 D, K106L, K108R, K108A, K131R, K170R, K175R, K192V, K192C, K216N, K216V, K229L, K237N, K238N, K240V, K243T, K246E, K248R, K249R, K273R, K275A, K287Q, K287C, K287A, K295A, K2951,K304L, K317R, K326A, and K400A (relative to SEQ ID NO: 1).

In particular embodiments, a variant and/or fragment of an ADI from Table A1.1-A1.2 retains all or a portion of the lysine substitutions designated in Table A2 and A3 below (as indicated for that particular sequence).

TABLE A2
Exemplary Substitutions Relative to ADI from wildtype M. columbinum (SEQ ID NO: 1)

| | 2 | 13 | 63 | 82 | 90 | 101 | 106 | 108 | 131 | 170 | 175 | 192 | 216 | 229 | 237 | 238 | 240 | 243 | 246 | 248 | 249 | 273 | 275 | 287 | 295 | 304 | 317 | 326 | 400 |
|---|---|---|---|---|---|---|---|---|---|---|---|---|---|---|---|---|---|---|---|---|---|---|---|---|---|---|---|---|---|
| Mcol | G | E | N | S | T | D | L | R | R | R | R | V | N | L | N | N | V | T | E | R | R | R | A | Q | A | L | R | A | A |
| M1 | | | | | | | | | | | | | | | | | | | | | | | | | | | | | |
| Mcol | G | | N | | T | | L | | R | | R | | N | | N | | V | | E | | R | | A | | A | | R | | A |
| M2 | | | | | | | | | | | | | | | | | | | | | | | | | | | | | |
| Mcol | G | E | | S | | D | | R | | R | | V | | L | | N | | T | | R | | R | | Q | | L | | A | |
| M3 | | | | | | | | | | | | | | | | | | | | | | | | | | | | | |
| Mcol | G | | | | T | | | | R | | | | N | | | | V | | | | R | | | | A | | | | A |
| M4 | | | | | | | | | | | | | | | | | | | | | | | | | | | | | |
| Mcol | G | | N | | | | L | | | | R | | | | N | | | | E | | | | A | | | | R | | |
| M5 | | | | | | | | | | | | | | | | | | | | | | | | | | | | | |
| Mcol | G | E | | | | D | | | | R | | | | L | | | | T | | | | R | | | | L | | | |
| M6 | | | | | | | | | | | | | | | | | | | | | | | | | | | | | |
| Mcol | G | | | S | | | | R | | | | V | | | | N | | | | R | | | | Q | | | | A | |
| M7 | | | | | | | | | | | | | | | | | | | | | | | | | | | | | |
| Mcol | G | | | S | T | | | R | R | | | V | N | | | N | V | | | R | R | | | Q | A | | | A | A |
| M8 | | | | | | | | | | | | | | | | | | | | | | | | | | | | | |
| Mcol | G | | | S | T | | | R | R | | R | V | N | | | N | V | | E | R | R | | | Q | A | | R | A | A |
| M9 | | | | | | | | | | | | | | | | | | | | | | | | | | | | | |
| Mcol | G | | | S | T | | L | R | R | | | V | N | | N | N | V | | | R | R | | A | Q | A | | | A | A |
| M10 | | | | | | | | | | | | | | | | | | | | | | | | | | | | | |
| Mcol | G | | | S | T | | | R | R | R | | V | N | | | N | V | T | | R | R | | | Q | A | L | | A | A |
| M11 | | | | | | | | | | | | | | | | | | | | | | | | | | | | | |
| Mcol | G | | | S | T | D | | R | R | | | V | N | L | | N | V | | | R | R | R | | Q | A | | | A | A |
| M12 | | | | | | | | | | | | | | | | | | | | | | | | | | | | | |
| Mcol | G | | | S | T | | | R | R | | | V | N | | | N | V | | E | R | R | | | Q | A | | R | A | A |
| M29 | | | | | | | | | | | | | | | | | | | | | | | | | | | | | |
| Mcol | G | | | S | T | | | R | R | | R | V | N | | | N | V | | | R | R | | | Q | A | | R | A | A |
| M30 | | | | | | | | | | | | | | | | | | | | | | | | | | | | | |
| Mcol | G | | | S | T | | | R | R | | R | V | N | | | N | V | | E | R | R | | | Q | A | | | A | A |
| M31 | | | | | | | | | | | | | | | | | | | | | | | | | | | | | |
| Mcol | G | | | S | T | | | R | R | | R | V | N | | | N | V | | | R | R | | | Q | A | | | A | A |
| M32 | | | | | | | | | | | | | | | | | | | | | | | | | | | | | |
| Mcol | G | | | S | T | | | R | R | | | V | N | | | N | V | | E | R | R | | | Q | A | | | A | A |
| M33 | | | | | | | | | | | | | | | | | | | | | | | | | | | | | |
| Mcol | G | | | S | T | | | R | R | | | V | N | | | N | V | | | R | R | | | Q | .A | | R | A | A |
| M34 | | | | | | | | | | | | | | | | | | | | | | | | | | | | | |
| Mcol | G | | | S | T | | | R | R | | | C | N | | | N | V | | | R | R | | | Q | A | | | A | A |
| M35 | | | | | | | | | | | | | | | | | | | | | | | | | | | | | |
| Mcol | G | | | S | T | | | R | R | | | V | N | | | N | V | | | R | R | | | C | A | | | A | A |
| M36 | | | | | | | | | | | | | | | | | | | | | | | | | | | | | |
| Mcol | G | | | S | T | | | R | R | | | C | N | | | N | V | | | R | R | | | C | A | | | A | A |
| M39 | | | | | | | | | | | | | | | | | | | | | | | | | | | | | |
| Mcol | G | | | S | T | | | R | R | | | V | N | | | N | V | | | R | R | | | Q | A | | K* | A | A |
| M46 | | | | | | | | | | | | | | | | | | | | | | | | | | | | | |
| Mcol | R | | | R | R | | | R | R | | | R | R | | | R | R | | R | R | R | | | R | R | | | R | R |
| M56 | | | | | | | | | | | | | | | | | | | | | | | | | | | | | |
| Mcol | | | | S | T | | | R | R | | | V | N | | | N | V | | E | R | R | | | Q | A | | | A | |
| M59 | | | | | | | | | | | | | | | | | | | | | | | | | | | | | |
| Mcol | | | | S | T | | | R | R | | | V | N | | | N | V | | E | R | R | | | Q | A | | | | |
| M60 | | | | | | | | | | | | | | | | | | | | | | | | | | | | | |
| Mcol | | | | S | T | | | R | R | | | V | N | | | M | V | | E | R | R | | | Q | | | | | |
| M61 | | | | | | | | | | | | | | | | | | | | | | | | | | | | | |
| Mcol | | | | S | T | | | R | R | | | V | N | | | N | V | | E | R | R | | | | | | | | |
| M62 | | | | | | | | | | | | | | | | | | | | | | | | | | | | | |
| Mcol | | | | | T | | | R | R | | | V | N | | | N | V | | E | R | R | | | | | | | | |
| M63 | | | | | | | | | | | | | | | | | | | | | | | | | | | | | |
| Mcol | | | | | T | | | R | R | | | V | N | | | N | V | | E | R | R | | | Q | A | | | | |
| M64 | | | | | | | | | | | | | | | | | | | | | | | | | | | | | |
| Mcol | | | | S | | | | R | R | | | V | N | | | N | V | | E | R | R | | | Q | A | | | | |
| M65 | | | | | | | | | | | | | | | | | | | | | | | | | | | | | |
| Mcol | | | | S | T | | | | R | | | V | N | | | N | V | | E | R | R | | | Q | A | | | | |
| M66 | | | | | | | | | | | | | | | | | | | | | | | | | | | | | |
| Mcol | | | | S | T | | | R | | | | V | N | | | N | V | | E | R | R | | | Q | A | | | | |
| M67 | | | | | | | | | | | | | | | | | | | | | | | | | | | | | |
| Mcol | | | | S | T | | | R | R | | | | N | | | N | V | | E | R | R | | | Q | A | | | | |
| M68 | | | | | | | | | | | | | | | | | | | | | | | | | | | | | |
| Mcol | | | | S | T | | | R | R | | | V | | | | N | V | | E | R | R | | | Q | A | | | | |
| M69 | | | | | | | | | | | | | | | | | | | | | | | | | | | | | |
| Mcol | | | | S | T | | | R | R | | | V | N | | | | V | | E | R | R | | | Q | A | | | | |
| M70 | | | | | | | | | | | | | | | | | | | | | | | | | | | | | |
| Mcol | | | | S | T | | | R | R | | | V | N | | | N | | | E | R | R | | | Q | A | | | | |
| M71 | | | | | | | | | | | | | | | | | | | | | | | | | | | | | |
| Mcol | | | | S | T | | | R | R | | | V | N | | | N | V | | E | | R | | | Q | A | | | | |
| M72 | | | | | | | | | | | | | | | | | | | | | | | | | | | | | |
| Mcol | | | | S | T | | | R | R | | | V | N | | | N | V | | E | R | | | | Q | A | | | | |
| M73 | | | | | | | | | | | | | | | | | | | | | | | | | | | | | |
| Mcol | | | | S | T | | | R | R | | | V | | | | | | | E | R | R | | | | A | | | | |
| M74 | | | | | | | | | | | | | | | | | | | | | | | | | | | | | |

*AAG codon was used instead of preferred AAA

TABLE A3
Modified Protein Amino Acid Substitutions and Measured Activity

| | McolM1 | McolM2 | McolM3 | McolM4 | McolM5 | McolM6 | McolM7 | McolM8 |
|---|---|---|---|---|---|---|---|---|
| | Refold | Refold | Refold | Soluble | Refold | Refold | Soluble | Soluble |
| % relative | ~17% | <80% | <80% | >80% | <80% | <80% | >80% | >80% |
| activity to | | | | | | | | |
| WT | | | | | | | | |
| | K2G | K2G | K2G | K2G | K2G | K2G | K2G | K2G |
| | K13E | | K13E | | | K13E | | |
| | K63N | K63N | | | K63N | | | |
| | K82S | | K82S | | | | K82S | K82S |
| | K90T | K90T | | K90T | | | | K90T |
| | K101D | | K101D | | | K101D | | |
| | K106L | K106L | | | K106L | | | |
| | K108R | | K108R | | | | K108R | K108R |
| | K131R | K131R | | K131R | | | | K131R |
| | K170R | | K170R | | | K170R | | |
| | K175R | K175R | | | K175R | | | |
| | K192V | | K192V | | | | K192V | K192V |
| | K216N | K216N | | K216N | | | | K216N |
| | K229L | | K229L | | | K229L | | |
| | K237N | K237N | | | K237N | | | |
| | K238N | | K238N | | | | K238N | K238N |
| | K240V | K240V | | K240V | | | | K240V |
| | K243T | | K243T | | | K243T | | |
| | K246E | K246E | | | K246E | | | |
| | K248R | | K248R | | | | K248R | K248R |
| | K249R | K249R | | K249R | | | | K249R |
| | K273R | | K273R | | | K273R | | |
| | K275A | K275A | | | K275A | | | |
| | K287Q | | K287Q | | | | K287Q | K287Q |
| | K295A | K295A | | K295A | | | | K295A |
| | K304L | | K304L | | | K304L | | |
| | K317R | K317R | | | K317R | | | |
| | K326A | | K326A | | | | K326A | K326A |

| McolM9 | McolM10 | McolM11 | McolM12 | McolM29 | McolM30 | McolM31 | McolM32 |
|---|---|---|---|---|---|---|---|
| Refold | Refold | Refold | Refold | Refold | Refold | Refold | Refold |
| >80% | >80% | <80% | <80% | >80% | <80% | >80% | <80% |
| K2G | K2G | K2G | K2G | K2G | K2G | K2G | K2G |
| K82S | K82S | K82S | K82S | K82S | K82S | K82S | K82S |
| K90T | K90T | K90T | K90T | K90T | K90T | K90T | K90T |
| | | | K101D | | | | |
| | K106L | | | | | | |
| K108R | K108R | K108R | K108R | K108R | K108R | K108R | K108R |
| K131R | K131R | K131R | K131R | K131R | K131R | K131R | K131R |
| | | K170R | | | | | |
| K175R | | | | | K175R | K175R | K175R |
| K192V | K192V | K192V | K192V | K192V | K192V | K192V | K192V |
| K216N | K216N | K216N | K216N | K216N | K216N | K216N | K216N |
| | | | K229L | | | | |
| | K237N | | | | | | |
| K238N | K238N | K238N | K238N | K238N | K238N | K238N | K238N |
| K240V | K240V | K240V | K240V | K240V | K240V | K240V | K240V |
| | | K243T | | | | | |
| K246E | | | | K246E | | K246E | |
| K248R | K248R | K248R | K248R | K248R | K248R | K248R | K248R |
| K249R | K249R | K249R | K249R | K249R | K249R | K249R | K249R |
| | | | K273R | | | | |
| | K275A | | | | | | |
| K287Q | K287Q | K287Q | K287Q | K287Q | K287Q | K287Q | K287Q |
| K295A | K295A | K295A | K295A | K295A | K295A | K295A | K295A |
| | | K304L | | | | | |
| K317R | | | | K317R | K317R | | |
| K326A | K326A | K326A | K326A | K326A | K326A | K326A | K326A |
| K400A | K400A | K400A | K400A | K400A | K400A | K400A | K400A |
| McolM33 | McolM34 | McolM35 | McolM36 | McolM39 | McolM46 | McolM56 | McolM59 |
| Refold | Refold | Refold | Refold | Refold | Refold | Soluble | Refold |
| >80% | >80% | >80% | >80% | >80% | >80% | ND | >80% |
| K2G | K2G | K2G | K2G | K2G | K2G | K2R | |
| K82S | K82S | K82S | K82S | K82S | K82S | K82R | K82S |
| K90T | K90T | K90T | K90T | K90T | K90T | K90R | K90T |
| K108R | K108R | K108R | K108R | K108R | K108R | K108R | K108R |
| K131R | K131R | K131R | K131R | K131R | K131R | K131R | K131R |
| K192V | K192V | K192C | K192V | K192C | K192V | K192R | K192V |
| K216N | K216N | K216N | K216N | K216N | K216N | K216R | K216N |
| K238N | K238N | K238N | K238N | K238N | K238N | K238R | K238N |
| K240V | K240V | K240V | K240V | K240V | K240V | K2490R | K240V |
| K246E | | | | | | K246E | K246E |
| K248R | K248R | K248R | K248R | K248R | K248R | K248R | K248R |
| K249R | K249R | K249R | K249R | K249R | K249R | K249R | K249R |
| K287Q | K287Q | K287Q | K287C | K287C | K287Q | K287R | K287Q |
| K295A | K295A | K295A | K295A | K295A | K295A | K295R | K295A |
| | K317R | | | | AAG* | | |
| K326A | K326A | K326A | K326A | K326A | K326A | K326R | K326A |
| K400A | K400A | K400A | K400A | K400A | K400A | K400R | |

| McolM60 | McolM61 | McolM62 | McolM63 | McolM64 | McolM65 | McolM66 |
|---|---|---|---|---|---|---|
| Refold | 50/50 | Soluble | Soluble | Refold | Soluble | Soluble |
| >80% | >80% | ND | ND | >80% | ND | ND |
| K82S | K82S | K82S | | | K82S | K82S |
| K90T | K90T | K90T | K90T | K90T | | K90T |
| K108R | K108R | K108R | K108R | K108R | K108R | |
| K131R | K131R | K131R | K131R | K131R | K131R | K131R |
| K192V | K192V | K192V | K192V | K192V | K192V | K192V |
| K216N | K216N | K216N | K216N | K216N | K216N | K216N |
| K238N | K238N | K238N | K238N | K238N | K238N | K238N |
| K240V | K240V | K240V | K240V | K240V | K240V | K240V |
| K246E | K246E | K246E | K246E | K246E | K246E | K246E |
| K248R | K248R | K248R | K248R | K248R | K248R | K248R |
| K249R | K249R | K249R | K249R | K249R | K249R | K249R |
| K287Q | K287Q | | | K287Q | K287Q | K287Q |
| K295A | | | | K295A | K295A | K295A |

| McolM67 | McolM68 | McolM69 | McolM70 | McolM71 | McolM72 | McolM73 | McolM74 |
|---|---|---|---|---|---|---|---|
| Refold | Soluble | Soluble | Refold | Soluble | Refold | Refold | Soluble |
| >80% | ND | ND | >80% | ND | >80% | >80% | ND |
| K82S | K82S | K82S | K82S | K82S | K82S | K82S | K82S |
| K90T | K90T | K90T | K90T | K90T | K90T | K90T | K90T |
| K108R | K108R | K108R | K108R | K108R | K108R | K108R | K108R |
| | K131R | K131R | K131R | K131R | K131R | K131R | K131R |
| K192V | | K192V | K192V | K192V | K192V | K192V | K192V |
| K216N | K216N | | K216N | K216N | K216N | K216N | K216N |
| K238N | K238N | K238N | | K238N | K238N | K238N | K238N |
| K240V | K240V | K240V | K240V | | K240V | K240V | K240V |
| K246E | K246E | K246E | K246E | K246E | K246E | K246E | K246E |
| K248R | K248R | K248R | K248R | K248R | | K248R | K248R |
| K249R | K249R | K249R | K249R | K249R | K249R | | K249R |
| K287Q | K287Q | K287Q | K287Q | K287Q | K287Q | K287Q | |
| K295A | K295A | K295A | K295A | K295A | K295A | K295A | K295A |
| McolM75 | McolM76 | McolM77 | McolM78 | McolM79 | McolM80 | McolM81 | McolM82 |
| Refold | Refold | Refold | Refold | Refold | Refold | Refold | Refold |
| >80% | >80% | >80% | >80% | >80% | >80% | >80% | >80% |
| AAG* | K82S | K82S | K82S | K82S | K82S | AAG* | K82S |
| K90T | K90T | K90T | K90T | K90T | K90T | K90T | K90T |
| K108R | K108R | K108R | K108R | K108R | K108R | K108R | K108R |
| K131R | AAG* | K131R | K131R | K131R | K131R | AAG* | AAG* |
| K192V | K192V | K192V | K192V | K192V | K192V | K192V | K192V |
| K216N | K216N | K216N | K216N | K216N | K216N | K216N | K216N |
| K240V | K240V | K240V | K240V | K240V | K240V | K240V | K240V |
| K246E | K246E | AAG* | K246E | K246E | K246E | K246E | AAG* |
| K248R | K248R | K248R | AAG* | K248R | K248R | K248R | K248R |
| K249R | K249R | K249R | K249R | AAG* | K249R | K249R | K249R |
| K287Q | K287Q | K287Q | K287Q | K287Q | K287Q | K287Q | K287Q |
| K295A | K295A | K295A | K295A | K295A | AAG* | K295A | K295A |
| McolM83 | McolM84 | McolM85 | McolM86 | McolM94 | McolM95 | McolM96 | McolM97 |
| Refold | Refold | Refold | Refold | <50% | <50% | <50% | >50% |
| | | | | Insoluble | Insoluble | Insoluble | Insoluble |
| >80% | >80% | >80% | >80% | ND | ND | ND | ND |
| | | | | K2G | K2G | K2G | K2G |
| AAG* | AAG* | AAG* | AAG* | K82S | K82S | K82S | K82S |
| K90T | K90T | K90T | K90T | K90T | K90T | K90T | K90T |
| K108R | K108R | K108R | K108R | K108R | K108R | K108R | K108R |
| K131R | AAG* | AAG* | AAG* | K131R | K131R | K131R | K131R |
| K192V | K192V | K192V | K192V | K192A | K192R | K192N | K192D |
| K216N | K216N | K216N | K216N | K216N | K216N | K216N | K216N |
| | | | | K238N | K238N | K238N | K238N |
| K240V | K240V | K240V | K240V | K240V | K240V | K240V | K240V |
| AAG* | AAG* | AAG* | AAG* | | | | |
| K248R | K248R | AAG* | AAG* | K248R | K248R | K248R | K248R |
| K249R | K249R | K249R | AAG* | K249R | K249R | K249R | K249R |
| K287Q | K287Q | K287Q | K287Q | K287Q | K287Q | K287Q | K287Q |
| K295A | K295A | K295A | K295A | K295A | K295A | K295A | K295A |
| | | | | K317R | K317R | K317R | K317R |
| | | | | K326A | K326A | K326A | K326A |
| | | | | K400A | K400A | K400A | K400A |

| McolM98 | McolM99 | McolM100 | McolM101 | McolM102 | McolM103 | McolM104 |
|---|---|---|---|---|---|---|
| <50% | >50% | Refold | >50% | <50% | <50% | <50% |
| Insoluble | Insoluble | | Insoluble | Insoluble | Insoluble | Insoluble |
| ND | ND | >80% | >80% | ND | ND | ND |
| K2G | K2G | K2G | K2G | K2G | K2G | K2G |
| K82S | K82S | K82S | K82S | K82S | K82S | K82S |
| K90T | K90T | K90T | K90T | K90T | K90T | K90T |
| K108R | K108R | K108R | K108R | K108R | K108R | K108R |
| K131R | K131R | K131R | K131R | K131R | K131R | K131R |
| K192Q | K192E | K192G | K192H | K192I | K192L | K192M |
| K216N | K216N | K216N | K216N | K216N | K216N | K216N |
| K238N | K238N | K238N | K238N | K238N | K238N | K238N |
| K240V | K240V | K240V | K240V | K240V | K240V | K240V |
| K248R | K248R | K248R | K248R | K248R | K248R | K248R |
| K249R | K249R | K249R | K249R | K249R | K249R | K249R |
| K287Q | K287Q | K287Q | K287Q | K287Q | K287Q | K287Q |
| K295A | K295A | K295A | K295A | K295A | K295A | K295A |
| K317R | K317R | K317R | K317R | K317R | K317R | K317R |
| K326A | K326A | K326A | K326A | K326A | K326A | K326A |
| K400A | K400A | K400A | K400A | K400A | K400A | K400A |

| McolM105 | McolM106 | McolM107 | McolM108 | McolM109 | McolM110 |
|---|---|---|---|---|---|
| <50% | <50% | Refold | >50% | >50% | <50% |
| Insoluble | Insoluble | | Insoluble | Insoluble | Insoluble |
| ND | ND | >80% | ND | ND | ND |
| K2G | K2G | K2G | K2G | K2G | K2G |
| K82S | K82S | K82S | K82S | K82S | K82S |
| K90T | K90T | K90T | K90T | K90T | K90T |
| K108R | K108R | K108R | K108R | K108R | K108R |
| K131R | K131R | K131R | K131R | K131R | K131R |
| K192S | K192T | K192V | K192V | K192V | K192V |
| K216N | K216N | K216N | K216N | K216N | K216N |
| K238N | K238N | K238N | K238N | K238N | K238N |
| K240V | K240V | K240V | K240V | K240V | K240V |
| K248R | K248R | K248R | K248R | K248R | K248R |
| K249R | K249R | K249R | K249R | K249R | K249R |
| K287Q | K287Q | K287A | K287R | K287N | K287D |
| K295A | K295A | K295A | K295A | K295A | K295A |
| K317R | K317R | K317R | K317R | K317R | K317R |
| K326A | K326A | K326A | K326A | K326A | K326A |
| K400A | K400A | K400A | K400A | K400A | K400A |

| McolM111 | McolM112 | McolM113 | McolM114 | McolM115 | McolM116 | McolM117 |
|---|---|---|---|---|---|---|
| >50% | Insoluble | <50% | Insoluble | Insoluble | Insoluble | >50% |
| Insoluble | | Insoluble | | | | Insoluble |
| ND | ND | ND | ND | ND | ND | ND |
| K2G | K2G | K2G | K2G | K2G | K2G | K2G |
| K82S | K82S | K82S | K82S | K82S | K82S | K82S |
| K90T | K90T | K90T | K90T | K90T | K90T | K90T |
| K108R | K108R | K108R | K108R | K108R | K108R | K108R |
| K131R | K131R | K131R | K131R | K131R | K131R | K131R |
| K192V | K192V | K192V | K192V | K192V | K192V | K192V |
| K216N | K216N | K216N | K216N | K216N | K216N | K216N |
| K238N | K238N | K238N | K238N | K238N | K238N | K238N |
| K240V | K240V | K240V | K240V | K240V | K240V | K240V |
| K248R | K248R | K248R | K248R | K248R | K248R | K248R |
| K249R | K249R | K249R | K249R | K249R | K249R | K249R |
| K287E | K287G | K287H | K287I | K287L | K287M | K287S |
| K295A | K295A | K295A | K295A | K295A | K295A | K295A |
| K317R | K317R | K317R | K317R | K317R | K317R | K317R |
| K326A | K326A | K326A | K326A | K326A | K326A | K326A |
| K400A | K400A | K400A | K400A | K400A | K400A | K400A |
| McolM118 | McolM119 | McolM120 | McolM121 | McolM122 | McolM123 | McolM124 |
| <50% | Refold | >50% | <50% | >50% | Refold | <50% |
| Insoluble | | Insoluble | Insoluble | Insoluble | | Insoluble |
| ND | >80% | ND | ND | ND | >80% | ND |
| K2G | K2G | K2G | K2G | K2G | K2G | K2G |
| K82S | K82S | K82S | K82S | K82S | K82S | K82S |
| K90T | K90T | K90A | K90R | K90N | K90D | K90Q |
| K108R | K108R | K108R | K108R | K108R | K108R | K108R |
| K131R | K131R | K131R | K131R | K131R | K131R | K131R |
| K192V | K192V | K192V | K192V | K192V | K192V | K192V |
| K216N | K216N | K216N | K216N | K216N | K216N | K216N |
| K238N | K238N | K238N | K238N | K238N | K238N | K238N |
| K240V | K240V | K240V | K240V | K240V | K240V | K240V |
| K248R | K248R | K248R | K248R | K248R | K248R | K248R |
| K249R | K249R | K249R | K249R | K249R | K249R | K249R |
| K287T | K287V | K287Q | K287Q | K287Q | K287Q | K287Q |
| K295A | K295A | K295A | K295A | K295A | K295A | K295A |
| K317R | K317R | K317R | K317R | K317R | K317R | K317R |
| K326A | K326A | K326A | K326A | K326A | K326A | K326A |
| K400A | K400A | K400A | K400A | K400A | K400A | K400A |

| McolM125 | McolM126 | McolM127 | McolM128 | McolM129 | McolM130 |
|---|---|---|---|---|---|
| >50% | Refold | >50% | Insoluble | >50% | >50% |
| Insoluble | | Insoluble | | Insoluble | Insoluble |
| ND | <80% | ND | ND | ND | ND |
| K2G | K2G | K2G | K2G | K2G | K2G |
| K82S | K82S | K82S | K82S | K82S | K82S |
| K90E | K90G | K90H | K90I | K90L | K90M |
| K108R | K108R | K108R | K108R | K108R | K108R |
| K131R | K131R | K131R | K131R | K131R | K131R |
| K192V | K192V | K192V | K192V | K192V | K192V |
| K216N | K216N | K216N | K216N | K216N | K216N |
| K238N | K238N | K238N | K238N | K238N | K238N |
| K240V | K240V | K240V | K240V | K240V | K240V |
| K248R | K248R | K248R | K248R | K248R | K248R |
| K249R | K249R | K249R | K249R | K249R | K249R |
| K287Q | K287Q | K287Q | K287Q | K287Q | K287Q |
| K295A | K295A | K295A | K295A | K295A | K295A |
| K317R | K317R | K317R | K317R | K317R | K317R |
| K326A | K326A | K326A | K326A | K326A | K326A |
| K400A | K400A | K400A | K400A | K400A | K400A |

| McolM131 | McolM132 | McolM133 | McolM134 | McolM135 | McolM136 | McolM137 |
|---|---|---|---|---|---|---|
| >50% | Refold | Refold | Insoluble | Insoluble | Insoluble | Insoluble |
| Insoluble | | | | | | |
| ND | >80% | >80% | ND | ND | ND | ND |
| K2G | K2G | K2G | K2G | K2G | K2G | K2G |
| K82S | K82S | K82S | K82S | K82S | K82S | K82S |
| K90S | K90V | K90T | K90T | K90T | K90T | K90T |
| K108R | K108R | K108A | K108N | K108D | K108Q | K108E |
| K131R | K131R | K131R | K131R | K131R | K131R | K131R |
| K192V | K192V | K192V | K192V | K192V | K192V | K192V |
| K216N | K216N | K216N | K216N | K216N | K216N | K216N |
| K238N | K238N | K238N | K238N | K238N | K238N | K238N |
| K240V | K240V | K240V | K240V | K240V | K240V | K240V |
| K248R | K248R | K248R | K248R | K248R | K248R | K248R |
| K249R | K249R | K249R | K249R | K249R | K249R | K249R |
| K287Q | K287Q | K287Q | K287Q | K287Q | K287Q | K287Q |
| K295A | K295A | K295A | K295A | K295A | K295A | K295A |
| K317R | K317R | K317R | K317R | K317R | K317R | K317R |
| K326A | K326A | K326A | K326A | K326A | K326A | K326A |
| K400A | K400A | K400A | K400A | K400A | K400A | K400A |
| McolM138 | McolM139 | McolM140 | McolM141 | McolM142 | McolM143 | McolM144 |
| Insoluble | >50% | Insoluble | Insoluble | Insoluble | Insoluble | Insoluble |
| | Insoluble | | | | | |
| ND | ND | ND | ND | ND | ND | ND |
| K2G | K2G | K2G | K2G | K2G | K2G | K2G |
| K82S | K82S | K82S | K82S | K82S | K82S | K82S |
| K90T | K90T | K90T | K90T | K90T | K90T | K90T |
| K108G | K108H | K108I | K108L | K108M | K108S | K108T |
| K131R | K131R | K131R | K131R | K131R | K131R | K131R |
| K192V | K192V | K192V | K192V | K192V | K192V | K192V |
| K216N | K216N | K216N | K216N | K216N | K216N | K216N |
| K238N | K238N | K238N | K238N | K238N | K238N | K238N |
| K240V | K240V | K240V | K240V | K240V | K240V | K240V |
| K248R | K248R | K248R | K248R | K248R | K248R | K248R |
| K249R | K249R | K249R | K249R | K249R | K249R | K249R |
| K287Q | K287Q | K287Q | K287Q | K287Q | K287Q | K287Q |
| K295A | K295A | K295A | K295A | K295A | K295A | K295A |
| K317R | K317R | K317R | K317R | K317R | K317R | K317R |
| K326A | K326A | K326A | K326A | K326A | K326A | K326A |
| K400A | K400A | K400A | K400A | K400A | K400A | K400A |

| McolM145 | McolM146 | McolM147 | McolM148 | McolM149 | McolM150 |
|---|---|---|---|---|---|
| Refold | <50% | <50% | >50% | Soluble | >50% |
| | Insoluble | Insoluble | Insoluble | | Insoluble |
| >80% | ND | ND | ND | ND | ND |
| K2G | K2G | K2G | K2G | K2G | K2G |
| K82S | K82S | K82S | K82S | K82S | K82S |
| K90T | K90T | K90T | K90T | K90T | K90T |
| K108V | K108R | K108R | K108R | K108R | K108R |
| K131R | K131R | K131R | K131R | K131R | K131R |
| K192V | K192V | K192V | K192V | K192V | K192V |
| K216N | K216A | K216R | K216D | K216Q | K216E |
| K238N | K238N | K238N | K238N | K238N | K238N |
| K240V | K240V | K240V | K240V | K240V | K240V |
| K248R | K248R | K248R | K248R | K248R | K248R |
| K249R | K249R | K249R | K249R | K249R | K249R |
| K287Q | K287Q | K287Q | K287Q | K287Q | K287Q |
| K295A | K295A | K295A | K295A | K295A | K295A |
| K317R | K317R | K317R | K317R | K317R | K317R |
| K326A | K326A | K326A | K326A | K326A | K326A |
| K400A | K400A | K400A | K400A | K400A | K400A |
| McolM151 | McolM152 | McolM153 | McolM154 | McolM155 | McolM156 |
| >50% | <50% | >50% | >50% | <50% | <50% |
| Insoluble | Insoluble | Insoluble | Insoluble | Insoluble | Insoluble |
| ND | ND | ND | ND | ND | ND |
| K2G | K2G | K2G | K2G | K2G | K2G |
| K82S | K82S | K82S | K82S | K82S | K82S |
| K90T | K90T | K90T | K90T | K90T | K90T |
| K108R | K108R | K108R | K108R | K108R | K108R |
| K131R | K131R | K131R | K131R | K131R | K131R |
| K192V | K192V | K192V | K192V | K192V | K192V |
| K216G | K216H | K216I | K216L | K216M | K216S |
| K238N | K238N | K238N | K238N | K238N | K238N |
| K240V | K240V | K240V | K240V | K240V | K240V |
| K248R | K248R | K248R | K248R | K248R | K248R |
| K249R | K249R | K249R | K249R | K249R | K249R |
| K287Q | K287Q | K287Q | K287Q | K287Q | K287Q |
| K295A | K295A | K295A | K295A | K295A | K295A |
| K317R | K317R | K317R | K317R | K317R | K317R |
| K326A | K326A | K326A | K326A | K326A | K326A |
| K400A | K400A | K400A | K400A | K400A | K400A |
| McolM157 | McolM158 | McolM159 | McolM160 | McolM161 | McolM162 |
| <50% | Refold | <50% | <50% | <50% | Refold |
| Insoluble | | Insoluble | Insoluble | Insoluble | |
| ND | >80% | ND | ND | ND | >80% |
| K2G | K2G | K2G | K2G | K2G | K2G |
| K82S | K82S | K82S | K82S | K82S | K82S |
| K90T | K90T | K90T | K90T | K90T | K90T |
| K108R | K108R | K108R | K108R | K108R | K108R |
| K131R | K131R | K131R | K131R | K131R | K131R |
| K192V | K192V | K192V | K192V | K192V | K192V |
| K216T | K216V | K216N | K216N | K216N | K216N |
| K238N | K238N | K238N | K238N | K238N | K238N |
| K240V | K240V | K240A | K240R | K240N | K240D |
| K248R | K248R | K248R | K248R | K248R | K248R |
| K249R | K249R | K249R | K249R | K249R | K249R |
| K287Q | K287Q | K287Q | K287Q | K287Q | K287Q |
| K295A | K295A | K295A | K295A | K295A | K295A |
| K317R | K317R | K317R | K317R | K317R | K317R |
| K326A | K326A | K326A | K326A | K326A | K326A |
| K400A | K400A | K400A | K400A | K400A | K400A |
| McolM163 | McolM164 | McolM165 | McolM166 | McolM167 | McolM168 |
| <50% | Insoluble | >50% | <50% | >50% | >50% |
| Insoluble | | Insoluble | Insoluble | Insoluble | Insoluble |
| ND | ND | ND | ND | ND | ND |
| K2G | K2G | K2G | K2G | K2G | K2G |
| K82S | K82S | K82S | K82S | K82S | K82S |
| K90T | K90T | K90T | K90T | K90T | K90T |
| K108R | K108R | K108R | K108R | K108R | K108R |
| K131R | K131R | K131R | K131R | K131R | K131R |
| K192V | K192V | K192V | K192V | K192V | K192V |
| K216N | K216N | K216N | K216N | K216N | K216N |
| K238N | K238N | K238N | K238N | K238N | K238N |
| K240Q | K240E | K240G | K240H | K240I | K240L |
| K248R | K248R | K248R | K248R | K248R | K248R |
| K249R | K249R | K249R | K249R | K249R | K249R |
| K287Q | K287Q | K287Q | K287Q | K287Q | K287Q |
| K295A | K295A | K295A | K295A | K295A | K295A |
| K317R | K317R | K317R | K317R | K317R | K317R |
| K326A | K326A | K326A | K326A | K326A | K326A |
| K400A | K400A | K400A | K400A | K400A | K400A |
| McolM169 | McolM170 | McolM171 | McolM172 | McolM173 | McolM174 |
| >50% | <50% | <50% | <50% | <50% | <50% |
| Insoluble | Insoluble | Insoluble | Insoluble | Insoluble | Insoluble |
| ND | ND | ND | ND | ND | ND |
| K2G | K2G | K2G | K2G | K2G | K2G |
| K82S | K82S | K82S | K82S | K82S | K82S |
| K90T | K90T | K90T | K90T | K90T | K90T |
| K108R | K108R | K108R | K108R | K108R | K108R |
| K131R | K131R | K131R | K131R | K131R | K131R |
| K192V | K192V | K192V | K192V | K192V | K192V |
| K216N | K216N | K216N | K216N | K216N | K216N |
| K238N | K238N | K238N | K238N | K238N | K238N |
| K240M | K240S | K240T | K240V | K240V | K240V |
| K248R | K248R | K248R | K248R | K248R | K248R |
| K249R | K249R | K249R | K249R | K249R | K249R |
| K287Q | K287Q | K287Q | K287Q | K287Q | K287Q |
| K295A | K295A | K295A | K295R | K295N | K295D |
| K317R | K317R | K317R | K317R | K317R | K317R |
| K326A | K326A | K326A | K326A | K326A | K326A |
| K400A | K400A | K400A | K400A | K400A | K400A |
| McolM175 | McolM176 | McolM177 | McolM178 | McolM179 | McolM180 |
| <50% | <50% | >50% | >50% | Refold | >50% |
| Insoluble | Insoluble | Insoluble | Insoluble | | Insoluble |
| ND | ND | ND | ND | >80% | ND |
| K2G | K2G | K2G | K2G | K2G | K2G |
| K82S | K82S | K82S | K82S | K82S | K82S |
| K90T | K90T | K90T | K90T | K90T | K90T |
| K108R | K108R | K108R | K108R | K108R | K108R |
| K131R | K131R | K131R | K131R | K131R | K131R |
| K192V | K192V | K192V | K192V | K192V | K192V |
| K216N | K216N | K216N | K216N | K216N | K216N |
| K238N | K238N | K238N | K238N | K238N | K238N |
| K240V | K240V | K240V | K240V | K240V | K240V |
| K248R | K248R | K248R | K248R | K248R | K248R |
| K249R | K249R | K249R | K249R | K249R | K249R |
| K287Q | K287Q | K287Q | K287Q | K287Q | K287Q |
| K295Q | K295E | K295G | K295H | K295I | K295L |
| K317R | K317R | K317R | K317R | K317R | K317R |
| K326A | K326A | K326A | K326A | K326A | K326A |
| K400A | K400A | K400A | K400A | K400A | K400A |
| McoM181 | McolM182 | McolM183 | McolM184 | McolM188 | McolM189 |
| >50% | <50% | <50% | >50% | Refold | Refold |
| Insoluble | Insoluble | Insoluble | Insoluble | | |
| ND | ND | ND | ND | <80% | <80% |
| K2G | K2G | K2G | K2G | | |
| K82S | K82S | K82S | K82S | | |
| K90T | K90T | K90T | K90T | K90V | AAG* |
| K108R | K108R | K108R | K108R | K108A | K108A |
| K131R | K131R | K131R | K131R | | |
| K192V | K192V | K192V | K192V | K192G | K192G |
| K216N | K216N | K216N | K216N | K216V | K216V |
| K238N | K238N | K238N | K238N | | |
| K240V | K240V | K240V | K240V | K240D | K240D |
| | | | | K246E | K246E |
| K248R | K248R | K248R | K248R | | |
| K249R | K249R | K249R | K249R | | |
| K287Q | K287Q | K287Q | K287Q | K287A | K287A |
| K295M | K295S | K295T | K295V | K295I | K295I |
| K317R | K317R | K317R | K317R | | |
| K326A | K326A | K326A | K326A | | |
| K400A | K400A | K400A | K400A | | |

| McolM190 | McolM191 | McolM192 | McolM193 | McolM194 | McolM195 | McolM196 |
|---|---|---|---|---|---|---|
| Refold | Refold | Refold | Refold | Refold | Refold | Soluble |
| <80% | <80% | <80% | <80% | <80% | <80% | ND |
| K90V | K90V | K90V | K90V | K90V | K90V | |
| AAG* | K108A | K108A | K108A | K108A | K108A | |
| K192G | AAG* | K192G | K192G | K192G | K192G | |
| K216V | K216V | AAG* | K216V | K216V | K216V | |
| K240D | K240D | K240D | AAG* | K240D | K240D | |
| K246E | K246E | K246E | K246E | K246E | K246E | K246E |
| K287A | K287A | K287A | K287A | AAG* | K287A | |
| K295I | K295I | K295I | K295I | K295I | AAG* | |
| McolM197 | McolM198 | McolM199 | McolM200 | McolM201 | McolM202 | McolM203 |
| Soluble | Soluble | Soluble | Soluble | Soluble | Soluble | Soluble |
| ND | ND | ND | ND | ND | ND | ND |
| K90V | | | | | | |
| | K108A | | | | | |
| | | K192G | | | | |
| | | | K216V | | | |
| | | | | K240D | | |
| K246E | K246E | K246E | K246E | K246E | K246E | K246E |
| | | | | | K287A | |
| | | | | | | K295I |

| McolM204 | McolM205 | McolM206 | McolM207 | McolM208 | McolM209 |
|---|---|---|---|---|---|
| Soluble | Soluble | Soluble | Soluble | Soluble | Soluble |
| ND | ND | ND | ND | ND | ND |
| K90V | K90V | K90V | K90V | K90V | K90V |
| K108A | | | | | |
| | K192G | | | | |
| | | K216V | | | |
| | | | K240D | | |
| K246E | K246E | K246E | K246E | K246E | K246E |
| | | | | K287A | |
| | | | | | K295I |
| McolM210 | McolM211 | McolM212 | McolM213 | McolM214 | McolM215 |
| Refold | Soluble | Soluble | Refold | Refold | Refold |
| <80% | ND | ND | <80% | >80% | <80% |
| K90V | K90V | K90V | K90V | K90V | K90V |
| K108A | | | | | K108A |
| | K192G | | | | K192G |
| | | K216V | | | |
| | | | K240D | | |
| K246E | K246E | K246E | K246E | K246E | K246E |
| | | | | K287A | |
| K295I | K295I | K295I | K295I | K295I | K295I |

| MclM216 | McolM217 | McolM218 | McolM219 | McolM220 | McolM221 | McolM222 |
|---|---|---|---|---|---|---|
| Refold | Refold | Refold | Refold | Refold | Refold | Soluble |
| >80% | <80% | >80% | <80% | <80% | <80% | ND |
| K90V | K90V | K90V | K90V | K90V | K90V | |
| K108A | K108A | K108A | K108A | K108A | K108A | K108A |
| | | | K192G | | | |
| K216V | | | | K216V | | K216V |
| | K240D | | | | K240D | |
| K246E | K246E | K246E | K246E | K246E | K246E | K246E |
| | | K287A | K287A | K287A | K287A | |
| K295I | K295I | K295I | K295I | K295I | K295I | K295I |

*AAG codon was used instead of the preferred AAA.
ND Not Determined
K246E enhances enzyme activity by ~5%
K106L, K170R, K175R, K229L, K275A, K317R reduce enzyme activity from 5-20% each.

In some instances, a variant comprises one or more “conservative” changes or substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present disclosure and still obtain a functional molecule that encodes a variant or derivative polypeptide with desirable characteristics. When it is desired to alter the amino acid sequence of a polypeptide to create an equivalent, or even an improved, variant or portion of a polypeptide described herein, one skilled in the art will typically change one or more of the codons of the encoding DNA sequence.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their utility.

In making such changes, the hydropathic index of amino acids may be considered. The importance of the hydropathic amino acid index in conferring interactive biologic function on a protein is generally understood in the art (Kyte & Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte & Doolittle, 1982). These values are: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred.

It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his.

A variant may also, or alternatively, contain non-conservative changes. In some embodiments, variant polypeptides differ from a native or reference sequence by substitution, deletion or addition of about or fewer than about 10, 9, 8, 7, 6, 5, 4, 3, 2 amino acids, or even 1 amino acid. Variants may also (or alternatively) be modified by, for example, the deletion or addition of amino acids that have minimal influence on the immunogenicity, secondary structure, enzymatic activity, and/or hydropathic nature of the polypeptide.

In certain embodiments, a polypeptide sequence is about, at least about, or up to about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 800, 810, 820, 830, 840, 850, 860, 870, 880, 890, 900, 900, 910, 920, 930, 940, 950, 960, 970, 980, 990, 1000 or more contiguous amino acids in length, including all integers in between, and which may comprise all or a portion of a reference sequence (see, e.g., Table A1.1 and A1.2, Sequence Listing).

In some embodiments, a polypeptide sequence consists of about or no more than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800. 800, 810, 820, 830, 840, 850, 860, 870, 880, 890, 900, 900, 910, 920, 930, 940, 950, 960, 970, 980, 990, 1000 or more contiguous amino acids, including all integers in between, and which may comprise all or a portion of a reference sequence (see, e.g., Table A1 and A1.2, Sequence Listing).

In certain embodiments, a polypeptide sequence is about 10-1000, 10-900, 10-800, 10-700, 10-600, 10-500, 10-400, 10-300, 10-200, 10-100, 10-50, 10-40, 10-30, 10-20, 20-1000, 20-900, 20-800, 20-700, 20-600, 20-500, 20-400, 20-300, 20-200, 20-100, 20-50, 20-40, 20-30, 50-1000, 50-900, 50-800, 50-700, 50-600, 50-500, 50-400, 50-300, 50-200, 50-100, 100-1000, 100-900, 100-800, 100-700, 100-600, 100-500, 100-400, 100-300, 100-200, 200-1000, 200-900, 200-800, 200-700, 200-600, 200-500, 200-400, or 200-300 contiguous amino acids, including all ranges in between, and comprises all or a portion of a reference sequence (see, e.g., Table A1, Sequence Listing). In certain embodiments, the C-terminal or N-terminal region of any reference polypeptide may be truncated by about 1, 2, 3, 4, 5, 6, 7, 8, 9, 10, 15, 20, 25, 30, 35, 40, 45, 50, 60, 70, 80, 90, 100, 110, 120, 130, 140, 150, 160, 170, 180, 190, 200, 250, 300, 350, 400, 450, 500, 550, 600, 650, 700, 750, or 800 or more amino acids, or by about 10-50, 20-50, 50-100, 100-150, 150-200, 200-250, 250-300, 300-350, 350-400, 400-450, 450-500, 500-550, 550-600, 600-650, 650-700, 700-750, 750-800 or more amino acids, including all integers and ranges in between (e.g., 101, 102, 103, 104, 105), so long as the truncated polypeptide retains the binding properties and/or activity of the reference polypeptide (see, e.g., Table A1.1 and A1.2, Sequence Listing). Typically, the biologically active fragment has no less than about 1%, about 5%, about 10%, about 25%, or about 50% of an activity of the biologically-active reference polypeptide from which it is derived.

In general, variants will display at least about 30%, 40%, 50%, 55%, 60%, 65%, 70%, 75%, 80%, 85%, 90%, 91%, 92%, 93%, 94%, 95%, 96%, 97%, 98%, 99% similarity or sequence identity or sequence homology to a reference polypeptide sequence (see, e.g., Table A1 and A1.2, Sequence Listing). Moreover, sequences differing from the native or parent sequences by the addition (e.g., C-terminal addition, N-terminal addition, both), deletion, truncation, insertion, or substitution (e.g., conservative substitution) of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 amino acids (including all integers and ranges in between) but which retain the properties or activities of a parent or reference polypeptide sequence are contemplated (see, e.g., Table A1.1 and A1.2, Sequence Listing.

In some embodiments, variant polypeptides differ from reference sequence by at least one but by less than 50, 40, 30, 20, 15, 10, 8, 6, 5, 4, 3 or 2 amino acid residue(s). In certain embodiments, variant polypeptides differ from a reference sequence by at least 1% but less than 20%, 15%, 10% or 5% of the residues. (If this comparison requires alignment, the sequences should be aligned for maximum similarity. “Looped” out sequences from deletions or insertions, or mismatches, are considered differences.)

Calculations of sequence similarity or sequence identity between sequences (the terms are used interchangeably herein) are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch, (J. Mol. Biol. 48: 444-453, 1970) algorithm which has been incorporated into the GAP program in the GCG software package, using either a Blossum 82 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the COG software package, using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller (Cabios. 4:11-17, 1989) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to nucleic acid molecules described herein. BLAST protein searches can be performed with the XBLAST program, score=50, wordlength=3 to obtain amino acid sequences homologous to protein molecules described herein. To obtain gapped alignments for comparison purposes, Gapped BLAST can be utilized as described in Altschul et al., (Nucleic Acids Res. 25: 3389-3402, 1997). When utilizing BLAST and Gapped BLAST programs, the default parameters of the respective programs (e.g., XBLAST and NBLAST) can be used.

In some embodiments, as noted above, polynucleotides and/or polypeptides can be evaluated using a BLAST alignment tool. A local alignment consists simply of a pair of sequence segments, one from each of the sequences being compared. A modification of Smith-Waterman or Sellers algorithms will find all segment pairs whose scores cannot be improved by extension or trimming, called high-scoring segment pairs (HSPs). The results of the BLAST alignments include statistical measures to indicate the likelihood that the BLAST score can be expected from chance alone.

The raw score, S, is calculated from the number of gaps and substitutions associated with each aligned sequence wherein higher similarity scores indicate a more significant alignment. Substitution scores are given by a look-up table (see PAM, BLOSUM).

Gap scores are typically calculated as the sum of G, the gap opening penalty and L, the gap extension penalty. For a gap of length n, the gap cost would be G+Ln. The choice of gap costs, G and L is empirical, but it is customary to choose a high value for G (10-15), e.g., 11, and a low value for L (1-2) e.g., 1.

The bit score, S′, is derived from the raw alignment score S in which the statistical properties of the scoring system used have been taken into account. Bit scores are normalized with respect to the scoring system, therefore they can be used to compare alignment scores from different searches. The terms “bit score” and “similarity score” are used interchangeably. The bit score gives an indication of how good the alignment is; the higher the score, the better the alignment.

The E-Value, or expected value, describes the likelihood that a sequence with a similar score will occur in the database by chance. It is a prediction of the number of different alignments with scores equivalent to or better than S that are expected to occur in a database search by chance. The smaller the E-Value, the more significant the alignment. For example, an alignment having an E value of e⁻¹¹⁷ means that a sequence with a similar score is very unlikely to occur simply by chance. Additionally, the expected score for aligning a random pair of amino acids is required to be negative, otherwise long alignments would tend to have high score independently of whether the segments aligned were related. Additionally, the BLAST algorithm uses an appropriate substitution matrix, nucleotide or amino acid and for gapped alignments uses gap creation and extension penalties. For example, BLAST alignment and comparison of polypeptide sequences are typically done using the BLOSUM62 matrix, a gap existence penalty of 11 and a gap extension penalty of 1.

In some embodiments, sequence similarity scores are reported from BLAST analyses done using the BLOSUM62 matrix, a gap existence penalty of 11 and a gap extension penalty of 1.

In a particular embodiment, sequence identity/similarity scores provided herein refer to the value obtained using GAP Version 10 (GCG, Accelrys, San Diego, Calif.) using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix (Henikoff and Henikoff, PNAS USA. 89:10915-10919, 1992). GAP uses the algorithm of Needleman and Wunsch (J Mol Biol. 48:443-453, 1970) to find the alignment of two complete sequences that maximizes the number of matches and minimizes the number of gaps.

In particular embodiments, the variant polypeptide comprises an amino acid sequence that can be optimally aligned with a reference polypeptide sequence (see, e.g., Table A1.1 and A1.2, Sequence Listing) to generate a BLAST bit scores or sequence similarity scores of at least about 50, 60, 70, 80, 90, 100, 100, 110, 120, 130, 140, 150, 180, 170, 180, 190, 200, 210, 220,230, 240, 250, 260, 270, 280, 290, 300, 310, 320, 330, 340, 350, 360, 370, 380, 390, 400, 410,420, 430, 440, 450, 460, 470, 480, 490, 500, 510, 520, 530, 540, 550, 560, 570, 580, 590, 600,610, 620, 630, 640, 650, 660, 670, 680, 690, 700, 710, 720, 730, 740, 750, 760, 770, 780, 790,800, 810, 820, 830, 840, 850, 860, 870, 880, 890, 900, 910, 920, 930, 940, 950, 960, 970, 980, 990, 1000, or more, including all integers and ranges in between, wherein the BLAST alignment used the BLOSUM62 matrix, a gap existence penalty of 11, and a gap extension penalty of 1.

As noted above, a reference polypeptide may be altered in various ways including amino acid substitutions, deletions, truncations, additions, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (PNAS USA. 82: 488-492, 1985); Kunkel et al., (Methods in Enzymol. 154: 367-382, 1987), U.S. Pat. No. 4,873,192, Watson, J. D. et al., (“Molecular Biology of the Gene,” Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

Methods for screening gene products of combinatorial libraries made by such modifications, and for screening cDNA libraries for gene products having a selected property are known in the art. Such methods are adaptable for rapid screening of the gene libraries generated by combinatorial mutagenesis of reference polypeptides. As one example, recursive ensemble mutagenesis (REM), a technique which enhances the frequency of functional mutants in the libraries, can be used in combination with the screening assays to identify polypeptide variants (Arkin and Yourvan, PNAS USA 89: 7811-7815, 1992; Delgrave et al., Protein Engineering. 6: 327-331, 1993).

In some embodiments, an isolated ADI is covalently bonded via an optional linker to at least one PEG molecule. In some instances, such molecules can be referred to as “ADI-PEG”; however, the term “ADI”, as used herein, is inclusive of “ADI-PEG”. “Polyethylene glycol” or “PEG” refers to mixtures of condensation polymers of ethylene oxide and water, in a branched or straight chain, represented by the general formula H(OCH₂CH₂)ₙOH, wherein n is at least 4. “Polyethylene glycol” or “PEG” is used in combination with a numeric suffix to indicate the approximate weight average molecular weight thereof. For example, PEG5,000 refers to PEG having a total weight average molecular weight of about 5,000; PEG12,000 refers to PEG having a total weight average molecular weight of about 12,000; and PEG20,000 refers to PEG having a total weight average molecular weight of about 20,000.

In some embodiments, the PEG has a total weight average molecular weight of about 1,000 to about 50,000; about 3,000 to about 40,000; about 5,000 to about 30,000; about 8,000 to about 30,000; about 11,000 to about 30,000; about 12,000 to about 28,000; about 16,000 to about 24,000; about 18,000 to about 22,000; or about 19,000 to about 21,000. In some embodiments, the PEG has a total weight average molecular weight of about 1,000 to about 50,000; about 3,000 to about 30,000; about 3,000 to about 20,000; about 4,000 to about 12,000; about 4,000 to about 10,000; about 4,000 to about 8,000; about 4,000 to about 6,000; or about 5,000. In specific embodiments, the PEG has a total weight average molecular weight of about 5,000 or about 20,000. Generally, PEG with a molecular weight of 30,000 or more is difficult to dissolve and yields of the formulated product may be reduced. The PEG may be a branched or straight chain. Generally, increasing the molecular weight of the PEG decreases the immunogenicity of the ADI. The PEG may be a branched or straight chain, and in certain embodiments is a straight chain. The PEG having a molecular weight described herein may be used in conjunction with ADI, and optionally, a biocompatible linker.

Certain embodiments employ thiol, sulfhydryl, or cysteine-reactive PEG(s). In some embodiments, the thiol, sulfhydryl, or cysteine-reactive PEG(s) are attached to one or more naturally-occurring cysteine residues, one or more introduced cysteine residues (e.g., substitution of one or more wild-type residues with cysteine residue(s)), insertion of one or more cysteine residues), or any combination thereof (see, e.g., Doherty et al., Bioconjug Chem. 16:1291-98, 2005). In certain embodiments, certain of the wild-type ADI cysteines residues may be first substituted with another amino acid to prevent attachment of the PEG polymer to wild-type cysteines, for example, to prevent the PEG(s) from disrupting an otherwise desirable biological activity. Some embodiments employ one or more non-natural cysteine derivatives (e.g., homocysteine) instead of cysteine.

Non-limiting examples of thiol, sulfhydryl, or cysteine-reactive PEGs include Methoxy PEG Maleimides (M-PEG-MAL) (e.g., MW 2000, MW 5000, MW 10000, MW 20000, MW 30000, MW 40000). M-PEG-MALs react with the thiol groups on cysteine side chains in proteins and peptides to generate a stable 3-thiosuccinimidyl ether linkage. This reaction is highly selective and can take place under mild conditions at about pH 5.0-6.5 in the presence of other functional groups. Thus, in certain embodiments, an ADI enzyme is conjugated to any one or more of the thiol, sulfhydryl, or cysteine-reactive PEG molecules described herein.

ADI may be covalently bonded to a modifying agent, such as PEG, with or without a linker, although a preferred embodiment utilizes a linker. ADI may be covalently bonded to PEG via a biocompatible linker using methods known in the art, as described, for example, by Park et al, Anticancer Res., 1:373-376 (1981); and Zaplipsky and Lee, Polyethylene Glycol Chemistry: Biotechnical and Biomedical Applications, J. M. Harris, ed., Plenum Press, NY, Chapter 21 (1992), the disclosures of which are hereby incorporated by reference herein in their entirety. In some instances, ADI may be coupled directly (i.e., without a linker) to a modifying agent such as PEG, for example, through an amino group, a sulfhydryl group, a hydroxyl group, a carboxyl group, or other group.

The linker used to covalently attach ADI to a modifying agent (e.g., PEG) can be any biocompatible linker. As discussed above, “biocompatible” indicates that the compound or group is non-toxic and may be utilized in vitro or in vivo without causing injury, sickness, disease, or death. A modifying agent such as PEG can be bonded to the linker, for example, via an ether bond, a thiol bond, an amide bond, or other bond.

In some embodiments, suitable linkers can have an overall chain length of about 1-100 atoms, 1-80 atoms, 1-60 atoms, 1-40 atoms, 1-30 atoms, 1-20 atoms, or 1, 2, 3, 4, 5, 6, 7, 8, 9, 10, 11, 12, 13, 14, 15, 16, 17, 18, 19, or 20 atoms, for example, wherein the atoms in the chain comprise C, S, N, P, and/or O. In certain embodiments, a linker is optional, e.g., a PEG conjugated ADI enzyme does not comprise a linker. In some instances, a linker group includes, for example, a succinyl group, an amide group, an imide group, a carbamate group, an ester group, an epoxy group, a carboxyl group, a hydroxyl group, a carbohydrate, a tyrosine group, a cysteine group, a histidine group, a methylene group, and combinations thereof. Particular examples of stable linkers include succinimide, propionic acid, carboxymethylate linkages, ethers, carbamates, amides, amines, carbamides, imides, aliphatic C—C bonds, and thio ethers. In certain embodiments, the biocompatible linker is a succinimidyl carboxymethyl ester (SCM) or N-hydroxy succinimide (NHS) group.

Other suitable linkers include an oxycarbonylimidazole group (including, for example, carbonylimidazole (CDI)), a nitro phenyl group (including, for example, nitrophenyl carbonate (NCP) or trichlorophenyl carbonate (TCP)), a trysylate group, an aldehyde group, an isocyanate group, a vinylsulfone group, or a primary amine. In certain embodiments, the linker is derived from SS, SPA, SCM, or NHS; in certain embodiments, SS, SPA, or NHS are used, and in some embodiments, SS or SPA are used. Thus, in certain embodiments, potential linkers can be formed from methoxy-PEG succinimidyl succinate (SS), methoxy-PEG succinimidyl glutarate (SG), methoxy-PEG succinimidyl carbonate (SC), methoxy-PEG succinimidyl carboxymethyl ester (SCM), methoxy-PEG2 N-hydroxy succinimide (NHS), methoxy-PEG succinimidyl butanoate (SBA), methoxy-PEG succinimidyl propionate (SPA), methoxy-PEG succinimidyl glutaramide, and/or methoxy-PEG succinimidyl succinimide.

Additional examples of linkers include, but are not limited to, one or more of the following: —O—, —NH—, —S—, —C(O)—, C(O)—NH, NH—C(O)—NH, O—C(O)—NH, —C(S)—, —CH2-, —CH2-CH2-, —CH2-CH2-CH2-, —CH2-CH2-CH2-CH2-, —O—CH2-, —CH2-O—, —O—CH2-CH2-, —CH2-O—CH2-, —CH2-CH2-O—, —O—CH2-CH2-CH2-, —CH2-O—CH2-CH2-, —CH2-CH2-O—CH2-, —CH2-CH2-CH2-O—, —O—CH2-CH2-CH2-CH2-, —CH2-O—CH2-CH2-CH2-, —CH2-CH2-O—CH2-CH2-, —CH2-CH2-CH2-O—CH2-, —CH2-CH2-CH2-CH2-O—, —C(O)—NH—CH2-, —C(O)—NH—CH2-CH2-, —CH2-C(O)—NH—CH2-, —CH2-CH2-C(O)—NH—, —C(O)—NH—CH2-CH2-CH2-, —CH2-C(O)—NH—CH2-CH2-, —CH2-CH2-C(O)—NH—CH2-, —CH2-CH2-CH2-C(O)—NH—, —C(O)—NH—CH2-CH2-CH2-CH2-, CH2-C(O)—NH—CH2-CH2-CH2-, —CH2-CH2-C(O)—NH—CH2-CH2-, —CH2-CH2-CH2-C(O)—NH—CH2-, —CH2-CH2-CH2-C(O)—NH—CH2-CH2-, —CH2-CH2-CH2-CH2-C(O)—NH—, —NH—C(O)—CH2-, CH2-NH—C(O)—CH2-, —CH2-CH2-NH—C(O)—CH2-, —NH—C(O)—CH2-CH2-, —CH2-NH—C(O)—CH2-CH2, —CH2-CH2-NH—C(O)—CH2-CH2, —C(O)—NH—CH2-, —C(O)—NH—CH2-CH2-, —O—C(O)—NH—CH2-, —O—C(O)—NH—CH2-CH2-, —NH—CH2-, —NH—CH2-CH2-, —CH2-NH—CH2-, —CH2-CH2-NH—CH2-, —C(O)—CH2-, —C(O)—CH2-CH2-, —CH2-C(O)—CH2-, —CH2-CH2-C(O)—CH2-, —CH2-CH2-C(O)—CH2-CH2-, —CH2-CH2-C(O)—, —CH2-CH2-CH2-C(O)—NH— CH2-CH2-NH—, —CH2-CH2-CH2-C(O)—NH—CH2-CH2-NH—C(O)—, —CH2-CH2-CH2-C(O)—NH—CH2-CH2-NH—C(O)—CH2-, bivalent cycloalkyl group, —N(R6)-, R6 is H or an organic radical selected from the group consisting of alkyl, substituted alkyl, alkenyl, substituted alkenyl, alkynyl, substituted alkynyl, aryl and substituted aryl.

Additionally, any of the linker moieties described herein may further include an ethylene oxide oligomer chain comprising 1 to 20 ethylene oxide monomer units [i.e., —(CH₂CH₂O)₁₋₂₀—]. That is, the ethylene oxide oligomer chain can occur before or after the linker, and optionally in between any two atoms of a linker moiety comprised of two or more atoms. Also, the oligomer chain would not be considered part of the linker moiety if the oligomer is adjacent to a polymer segment and merely represent an extension of the polymer segment.

In certain embodiments, the ADI enzyme comprises one or more PEG molecules and/or linkers as described herein. In certain embodiments, the linker is a water-labile linker.

The attachment of PEG to ADI increases the circulating half-life of ADI. Generally, PEG is attached to a primary amine of ADI. Selection of the attachment site of PEG, or other modifying agent, on the ADI is determined by the role of each of the sites within the active domain of the protein, as would be known to the skilled artisan. PEG may be attached to the primary amines of ADI without substantial loss of enzymatic activity. For example, the lysine residues present in ADI are all possible points at which ADI as described herein can be attached to PEG via a biocompatible linker, such as SS, SPA, SCM, SSA and/or NHS. PEG may also be attached to other sites on ADI, as would be apparent to one skilled in the art in view of the present disclosure.

From 1 to about 30 PEG molecules may be covalently bonded to ADI. In certain embodiments, ADI is modified with (i.e., comprises) one PEG molecule. In some embodiments, the ADI is modified with more than one PEG molecule. In particular embodiments, the ADI is modified with about 1 to about 10, or from about 7 to about 15 PEG molecules, or from about 2 to about 8 or about 9 to about 12 PEG molecules. In some embodiments, the ADI is modified with about 2, 3, 4, 5, 6, 7, 8, 9, 10, 11, 12, 13, 14, or 15 PEG molecules. In specific embodiments, ADI is modified with 4.5-5.5 PEG molecules per ADI. In some embodiment, ADI is modified with 5±1.5 PEG molecules. In some embodiments, an isolated ADI is covalently bonded to about 1 to about 10 PEG molecules. In some embodiments, an isolated ADI is covalently bonded to about 2 to about 8 PEG molecules.

In certain embodiments, about 15% to about 70% of the primary amino groups in ADI are modified with PEG. For instance, in some embodiments about 15% to about 60%, about 15% to about 50%, about 15% to about 40%, about 15% to about 30%, about 15% to about 25%, about 15% to about 20%, about 20% to about 60%, about 20% to about 50%, about 20% to about 40%, about 20% to about 30%, or about 20% to about 25% of the primary amino groups in arginine deiminase are modified with PEG. In specific embodiments about 20% of the primary amino groups in arginine deiminase are modified with PEG. When PEG is covalently bonded to the end terminus of ADI, it may be desirable to have only 1 PEG molecule utilized. Increasing the number of PEG units on ADI increases the circulating half-life of the enzyme. However, increasing the number of PEG units on ADI decreases the specific activity of the enzyme. Thus, a balance needs to be achieved between the two, as would be apparent to one skilled in the art in view of the present disclosure.

In some embodiments, a common feature of biocompatible linkers is that they attach to a primary amine of arginine deiminase via a succinimide group. Once coupled with ADI, SS-PEG has an ester linkage next to the PEG, which may render this site sensitive to serum esterase, which may release PEG from ADI in the body. SPA-PEG and PEG2-NHS do not have an ester linkage, so they are not sensitive to serum esterase.

PEG which is attached to the protein may be either a straight chain, as with SS-PEG, SPA-PEG and SC-PEG, or a branched chain of PEG may be used, as with PEG2-NHS.

In some embodiments, for example, the amino acid substitutions employ non-natural amino acids for conjugation to PEG or other modifying agent (see, e.g., de Graaf et al., Bioconjug Chem. 20:1281-95, 2009). Certain embodiments thus include an ADI enzyme that is conjugated to one or more PEGs via one or more non-natural amino acids. In some embodiments the non-natural amino acid comprises a side chain having a functional group selected from the group consisting of: an alkyl, aryl, aryl halide, vinyl halide, alkyl halide, acetyl, ketone, aziridine, nitrile, nitro, halide, acyl, keto, azido, hydroxyl, hydrazine, cyano, halo, hydrazide, alkenyl, alkynyl, ether, thio ether, epoxide, sulfone, boronic acid, boronate ester, borane, phenylboronic acid, thiol, seleno, sulfonyl, borate, boronate, phospho, phosphono, phosphine, heterocyclic-, pyridyl, naphthyl, benzophenone, a constrained ring such as a cyclooctyne, thioester, enone, imine, aldehyde, ester, thioacid, hydroxylamine, amino, carboxylic acid, alpha-keto carboxylic acid, alpha or beta unsaturated acids and amides, glyoxyl amide, and an organosilane group. In some embodiments, the non-natural amino acid is selected from the group consisting of: p-acetyl-L-phenylalanine, O-methyl-L-tyrosine, L-3-(2-naphthyl)alanine, 3-methyl-phenylalanine, O-4-allyl-L-tyrosine, homocysteine, 4-propyl-L-tyrosine, tri-O-acetyl-GlcNAcß-serine, ß-O-GlcNAc-L-serine, tri-O-acetyl-GalNAc-a-threonine, a GalNAc-L-threonine, L-Dopa, a fluorinated phenylalanine, isopropyl-L-phenylalanine, p-azido-L-phenylalanine, p-acyl-L-phenylalanine, p-benzoyl-L-phenylalanine, L-phosphoserine, phosphonoserine, phosphonotyrosine, p-iodo-phenylalanine, p-bromophenylalanine, p-amino-L-phenylalanine, and isopropyl-L-phenylalanine.

While ADI-PEG is the illustrative modified ADI described herein, as would be recognized by the skilled person ADI may be modified with other polymers or appropriate molecules for the desired effect, in particular reducing antigenicity and increasing serum half-life.

Compositions and Methods of Use

Certain embodiments include therapeutic compositions comprising an ADI protein described herein, and methods of using the same for arginine depletion therapies, including the treatment of various cancers.

For example, certain embodiments include compositions, for example, therapeutic or pharmaceutical compositions, comprising an isolated ADI described herein, and a pharmaceutically-acceptable carrier. Certain compositions are substantially pure on a protein basis or weight-basis. For instance, certain compositions have a purity of at least about 80%, 85%, 90%, 95%, 98%, or 99% on a protein basis or a weight-weight basis and are substantially aggregate-free, for example, less than about 10, 9, 8, 7, 6, or 5% aggregated. Certain compositions are substantially endotoxin-free, as described herein.

The compositions may be prepared by methodology well known in the pharmaceutical art. For example, a composition intended to be administered by injection can be prepared by combining a composition that comprises an isolated ADI, as described herein, and optionally one or more of buffers or excipients, optionally with sterile, distilled water so as to form a solution. A surfactant can be added to facilitate the formation of a homogeneous solution or suspension. Surfactants are compounds that non-covalently interact with the ADI in the composition so as to facilitate dissolution or homogeneous suspension of the ADI in the aqueous delivery system.

Some embodiments comprise a pharmaceutically-acceptable buffer, for example, a buffer selected from one or more of histidine, sodium citrate, glycyl-glycine, sodium phosphate, Tris, and lysine. In certain embodiments, the buffer is at a concentration of about 0.10 mM to about 200 mM, or about 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.0, 1.5, 2.0, 2.5, 3.0, 3.5, 4.0, 4.5, 5.0, 5.5, 6.0, 6.5. 7.0, 7.5, 8.0, 8.5, 9.0, 9.5, 10, 11, 12, 13, 14, 15, 16, 17, 18, 19, 20, 25, 30, 35, 40, 45, 50, 55, 60, 65, 70, 75, 80, 85, 90, 95, 100, 105, 110, 120, 125, 130, 135, 140, 145, 150, 155, 160, 165, 170, 175, 180, 185, 190, 195, or 200 mM, including all integers and ranges in between. In particular embodiments, the buffer is at about 1 to about 50 mM, or about 10 to about 30 mM, or about 15 to about 25 mM, or about 20 mM, or about 10 mM.

Some embodiments comprise a pharmaceutically-acceptable excipient, for example, an excipient selected from one or more of a cryoprotectant, a lyoprotectant, a stabilizer, a bulking agent, a tonicity modifier, a surfactant, a pharmaceutical plasticizer, a chelator, and any combination of the foregoing.

In some embodiments, the cryoprotectant is present at about 0.001% to about 20% (wt %), including all integers and ranges in between. In some embodiments, the cryoprotectant is selected from one or more of sucrose, trehalose, ethylene glycol, propylene glycol, glycerol, and any combination of the foregoing.

In some embodiments, the lyoprotectant is present at about 0.001% to about 20% (wt %), including all integers and ranges in between. In some embodiments, the lyoprotectant is selected from one or more of sucrose, trehalose, mannitol, sorbitol, glycerol, and any combination of the foregoing.

In some embodiments, the stabilizer is present at about 0.001% to about 20% (wt %), including all integers and ranges in between. In certain embodiments, the stabilizer is selected from one or more of sucrose, mannitol, lactose, trehalose, maltose, sorbitol, gelatin, albumin, and any combination of the foregoing.

In certain embodiments, the bulking agent is present at about 0.001% to about 20% (wt %), including all integers and ranges in between. In certain embodiments, the bulking agent is selected from one or more of mannitol, sorbitol, lactose, glucose, sucrose, glycine, albumin, dextran 40.

In certain embodiments, the tonicity modifier is present at about 0.001% to about 20% (wt %), including all integers and ranges in between. In particular embodiments, the tonicity modifier is selected from one or more of sodium chloride, sucrose, mannitol, and any combination of the foregoing.

Particular compositions comprise a pharmaceutically-acceptable excipient selected from one or more of sucrose, trehalose, dextran, mannitol, proline, glycine, a surfactant, a pharmaceutical plasticizer, a chelator, and any combination of the foregoing.

Certain compositions comprise a chelator, for example, ethylenediaminetetraacetic acid (EDTA). In some embodiments, the chelator is present at about 0.001% to about 1% (wt %), or about 0.001, 0.002, 0.003, 0.004, 0.005, 0.006, 0.007, 0.008, 0.009, 0.010, 0.015, 0.020, 0.025, 0.030, 0.035, 0.040, 0.045, 0.050, 0.055, 0.060, 0.065, 0.070, 0.075, 0.080, 0.085, 0.090, 0.095, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, or 1.0%, including all integers and ranges in between.

Certain compositions are at a pharmaceutically-acceptable pH. For instance, in certain embodiments, the pharmaceutically-acceptable pH is about 5.0 to about 8.0 (±0.01 to ±0.1), or about 5.0, 5.1, 5.2, 5.3, 5.4, 5.5, 5.6, 5.7, 5.8, 5.9, 6.0, 6.1. 6.2. 6.3. 6.4. 6.5. 6.6. 6.7. 6.8. 6.9. 7.0, 7.1, 7.2, 7.3, 7.4, 7.5, 7.6, 7.7, 7.8, 7.9, or 8.0 (±0.01 to ±0.1), including all integers and ranges in between.

In certain embodiments, an ADI has ADI activity at a pH close to the physiological pH of human blood. Thus, in some embodiments, an ADI has ADI activity at a pH of about 4 to about 10.8, or about 6 to about 8, or about 6.5 to about 7.5. In certain embodiments, an ADI has good ADI enzyme activity at about pH 7.4.

In certain embodiments, an ADI has stability during long term storage and temperature and proteolytic stability during treatment in the human body. In some embodiments, an ADI does not require ions or cofactors for activity that are not already present in blood.

In some embodiments, an isolated ADI, or a composition comprising the same, has an osmolality of about 50 mOsm/kg to about 500 mOsm/kg, or about 50, 55, 60, 65, 70, 75, 80, 85, 90, 95, 100, 105, 110, 120, 125, 130, 135, 140, 145, 150, 155, 160, 165, 170, 175, 180, 185, 190, 195, 200, 205, 210, 220, 225, 230, 235, 240, 245, 250, 255, 260, 265, 270, 275, 280, 285, 290, 295, 300, 305, 310, 320, 325, 330, 335, 340, 345, 350, 355, 360, 365, 370, 375, 380, 385, 390, 395, 400, 405, 410, 420, 425, 430, 435, 440, 445, 450, 455, 460, 465, 470, 475, 480, 485, 490, 495, or about 500 mOsm/kg.

In some embodiments, the specific ADI enzyme activity of a composition is between about 5.0 and 120 IU/mg, where 1 IU is defined as the amount of enzyme that converts one μmol of arginine into one μmol of citrulline and 1 μmol of ammonia in one minute at 37° C. and the potency is 100±20 IU/mL. In certain embodiments, the specific enzyme activity is about 5.5, 6, 6.5, 7, 7.5, 8, 8.5, 9.0, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 35, 40, 45, 50, 55, 55, 60, 65, 70, 75, 80, 85, 90, 95, 100, 105, 110, 115, or 120 IU/mg.

In certain embodiments, the ADI protein concentration is between about 5-20 mg/mL, for example, about 5, 6, 7, 8, 9, 10, 11, 12, 13, 14, 15, 16, 17, 18, 19, or 20 mg/mL.

In specific embodiments, the composition has one or more of the following determinations of purity: less than about 1 EU endotoxin/mg protein, less that about 100 ng host cell protein/mg protein, less than about 10 pg host cell DNA/mg protein, and/or greater than about 95% single peak purity by SEC HPLC.

Also included are methods of treating, ameliorating the symptoms of, or inhibiting the progression of, a cancer in a subject in need thereof, comprising administering to the subject a composition comprising at least one isolated ADI, as described herein.

The methods and compositions described herein can be used in the treatment of any variety of cancers. In some embodiments, the cancer is selected from one or more of hepatocellular carcinoma (HCC), melanoma, metastatic melanoma, pancreatic cancer, prostate cancer, small cell lung cancer, mesothelioma, lymphocytic leukemia, chronic myelogenous leukemia, lymphoma, hepatoma, sarcoma, leukemia, acute myeloid leukemia, relapsed acute myeloid leukemia, B-cell malignancy, breast cancer, ovarian cancer, colorectal cancer, gastric cancer, glioma (e.g., astrocytoma, oligodendroglioma, ependymoma, or a choroid plexus papilloma), glioblastoma multiforme (e.g., giant cell gliobastoma or a gliosarcoma), meningioma, pituitary adenoma, vestibular schwannoma, primary CNS lymphoma, primitive neuroectodermal tumor (medulloblastoma), non-small cell lung cancer (NSCLC), kidney cancer, bladder cancer, uterine cancer, esophageal cancer, brain cancer, head and neck cancers, cervical cancer, testicular cancer, and stomach cancer.

In some embodiments, the cancer exhibits reduced expression and/or activity of argininosuccinate synthetase-1 (ASS-1), or is otherwise argininosuccinate synthetase-1-deficient. In some instances, reduced ASS-1 expression or activity is a reduction in expression and/or activity of about 5%, 10%, 15%, 20%, 25%, 30%, 35%, 40%, 45%, 50%, 55%, 60%, 65%, 70%, or more, relative to expression and/or activity in an appropriate control sample, for example, a normal cell or tissue. In certain embodiments, ASS or ASL expression or activity is reduced by at least two-fold relative to expression or activity in a control sample. Reduction in ASS-1 expression or activity can be measured according to routine techniques the art, including, for example, quantitative PCR, immunohistochemistry, enzyme activity assays (e.g., ADI activity assays to measure conversion of citrulline into argininosuccinate or conversion of argininosuccinate into arginine and fumarate), and the like.

Administration may be achieved by a variety of different routes. Modes of administration depend upon the nature of the condition to be treated or prevented. For example, an ADI can be administered orally, intranasally, intraperitoneally, parenterally, intravenously, intralymphatically, intratumorally, intramuscularly, interstitially, intra-arterially, subcutaneously, intraocularly, intrasynovial, transepithelial, and/or transdermally. Particular embodiments include administration by IV infusion. An amount that, following administration, reduces, inhibits, prevents, or delays the growth, progression, and/or metastasis of a cancer is considered effective.

In some embodiments, the methods or compositions described herein increase median survival time of a patient by 4 weeks, 5 weeks, 6 weeks, 7 weeks, 8 weeks, 9 weeks, 10 weeks, 15 weeks, 20 weeks, 25 weeks, 30 weeks, 40 weeks, or longer. In certain embodiments, the methods or compositions described herein increase median survival time of a patient by 1 year, 2 years, 3 years, or longer. In some embodiments, the methods or compositions described herein increase progression-free survival by 2 weeks, 3 weeks, 4 weeks, 5 weeks, 6 weeks, 7 weeks, 8 weeks, 9 weeks, 10 weeks or longer. In certain embodiments, the methods or compositions described herein increase progression-free survival by 1 year, 2 years, 3 years, or longer.

In certain embodiments, the composition administered is sufficient to result in tumor regression, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the composition administered is sufficient to result in stable disease. In certain embodiments, the composition administered is sufficient to result in stabilization or clinically relevant reduction in symptoms of a particular disease indication known to the skilled clinician.

The methods or compositions for treating cancers can be combined with other therapeutic modalities. For example, a composition described herein can be administered to a subject before, during, or after other therapeutic interventions, including symptomatic care, radiotherapy, surgery, transplantation, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

Methods for identifying subjects with one or more of the diseases or conditions described herein are known in the art.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

In some embodiments, a therapeutically effective amount or therapeutic dosage of a composition described herein is an amount that is effective to reduce or stabilize tumor growth.

In certain instances, treatment is initiated with small dosages which can be increased by small increments until the optimum effect under the circumstances is achieved.

In some embodiments, a dosage is administered from about once a day to about once every two or three weeks. For example, in certain embodiments, a dosage is administered about once every 1, 2, 3, 4, 5, 6, or 7 days, or about once a week, or about twice a week, or about three times a week, or about once every two or three weeks.

In some embodiments, the dosage is from about 0.1 mg/kg to about 20 mg/kg, or to about 10 mg/kg, or to about 5 mg/kg, or to about 3 mg/kg. In some embodiments, the dosage is about 0.10 mg/kg, 0.15 mg/kg, 0.20 mg/kg, 0.25 mg/kg, 0.30 mg/kg, 0.35 mg/kg, 0.40 mg/kg, 0.45 mg/kg, 0.50 mg/kg, 0.55 mg/kg, 0.60 mg/kg, 0.65 mg/kg, 0.70 mg/kg, 0.75 mg/kg, 0.80 mg/kg, 0.85 mg/kg, 0.90 mg/kg, 0.95 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, 5.0 mg/kg, 5.5 mg/kg, 6.0 mg/kg, 6.5 mg/kg. 7.0 mg/kg, 7.5 mg/kg, 8.0 mg/kg, 8.5 mg/kg, 9.0 mg/kg, 9.5 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg, including all integers and ranges in between. In specific embodiments, the dosage is about 1 mg/kg once a week as a 2 ml intravenous injection to about 20 mg/kg once every 3 days.

In some embodiments, the dosage is from about 50 IU/m² to about 1000 IU/m². In particular embodiments, the dosage is about 50 IU/m², 60 IU/m², 70 IU/m², 80 IU/m², 90 IU/m², 100 IU/m², 110 IU/m², 120 IU/m², 130 IU/m², 140 IU/m², 150 IU/m², 160 IU/m², 170 IU/m², 180 IU/m², 190 IU/m², 200 IU/m², 210 IU/m², 220 IU/m², 230 IU/m², 240 IU/m², 250 IU/m², 260 IU/m², 270 IU/m², 280 IU/m², 290 IU/m², 300 IU/m², 310 IU/m², about 320 IU/m², about 330 IU/m², 340 IU/m² about 350 IU/m², 360 IU/m², 370 IU/m², 380 IU/m², 390 IU/m², 400 IU/m², 410 IU/m², 420 IU/m², 430 IU/m², 440 IU/m², 450 IU/m², 500 IU/m², 550 IU/m², 600 IU/m², 620 IU/m², 630 IU/m², 640 IU/m², 650 IU/m², 660 IU/m², 670 IU/m², 680 IU/m², 690 IU/m², 700 IU/m², 710 IU/m², 720 IU/m², 730 IU/m², 740 IU/m², 750 IU/m², 760 IU/m², 770 IU/m², 780 IU/m², 790 IU/m², 800 IU/m², 810 IU/m², 820 IU/m², 830 IU/m², 840 IU/m². 850 IU/m², 860 IU/m², 870 IU/m², 880 IU/m², 890 IU/m², 900 IU/m², 910 IU/m², 920 IU/m², 930 IU/m², 940 IU/m², 950 IU/m², 960 IU/m², 970 IU/m², 980 IU/m², 990 IU/m², or about 1000 IU/m², including all integers and ranges in between.

Also included are patient care kits, comprising one or more compositions or isolated ADIs described herein. Certain kits also comprise one or more pharmaceutically-acceptable diluents or solvents, such as water (e.g., sterile water). In some embodiments, the compositions or ADIs are stored in vials, cartridges, dual chamber syringes, and/or pre-filled mixing systems.

The kits herein may also include a one or more additional therapeutic agents or other components suitable or desired for the indication being treated, or for the desired diagnostic application. The kits herein can also include one or more syringes or other components necessary or desired to facilitate an intended mode of delivery (e.g., stents, implantable depots, etc.).

Polynucleotides, Expression Vectors, and Host Cells

Certain embodiments relate to polynucleotides that encode a modified ADI protein, as described herein. Thus, certain embodiments include a polynucleotide that encodes any one or more of the individual ADI polypeptides in Table A1.1 and A1.2 (excluding SEQ ID NO:1), including variants and/or fragments thereof. For instance, certain polynucleotides encode an ADI protein that comprises, consists, or consists essentially of an amino acid sequence that is at least 90, 95, 96, 97, 98, 99, or 100% identical to a reference amino sequence selected from Table A1.1 and A1.2 (e.g., SEQ ID NOs: 2-178), excluding SEQ ID NO:1.

In some embodiments, a polynucleotide comprises at least one AAG codon that encodes a lysine residue (rather than the preferred AAA codon), for example, an AAG codon that encodes the K317 residue. Some polynucleotides comprise at least about 1, 2, 3, 4, 5, 6, 7, 8, 9, 10, 11, 12, 13, 14, 15, 16, 17, 18, 19, or 20 AAG codons that encode a lysine residue ((rather than the preferred AAA codon).

Among other uses, these and related embodiments may be utilized to recombinantly produce an ADI protein in a host cell. It will be appreciated by those of ordinary skill in the art that, as a result of the degeneracy of the genetic code, there are many nucleotide sequences that encode a polypeptide described herein. Some of these polynucleotides may bear minimal homology to the nucleotide sequence of any native gene. Nonetheless, polynucleotides that vary due to differences in codon usage are specifically contemplated, for example, polynucleotides that are optimized for human, yeast or bacterial codon selection.

As will be recognized by the skilled artisan, polynucleotides may be single-stranded (coding or antisense) or double-stranded, and may be DNA (genomic, cDNA or synthetic) or RNA molecules. Polynucleotides may comprise a native sequence (i.e., an endogenous sequence that encodes an ADI protein) or may comprise a variant, or a biological functional equivalent of such a sequence. Polynucleotide variants may contain one or more substitutions, additions, deletions and/or insertions, as described herein, preferably such that the activity of the variant polypeptide is not substantially diminished relative to the unmodified polypeptide.

Additional coding or non-coding sequences may, but need not, be present within a polynucleotide, and a polynucleotide may, but need not, be linked to other molecules and/or support materials. Hence, the polynucleotides, regardless of the length of the coding sequence itself, may be combined with other DNA or RNA sequences, such as promoters, enhances, polyadenylation signals, additional restriction enzyme sites, multiple cloning sites, other coding segments, and the like, such that their overall length may vary considerably.

The polynucleotide sequences may also be of mixed genomic, cDNA, RNA, and that of synthetic origin. For example, a genomic or cDNA sequence encoding a leader peptide may be joined to a genomic or cDNA sequence encoding the polypeptide, after which the DNA or RNA sequence may be modified at a site by inserting synthetic oligonucleotides encoding the desired amino acid sequence for homologous recombination in accordance with well-known procedures or preferably generating the desired sequence by PCR using suitable oligonucleotides. In some embodiments a signal sequence can be included before the coding sequence. This sequence encodes a signal peptide N-terminal to the coding sequence which communicates to the host cell to direct the polypeptide to the cell surface or secrete the polypeptide into the media. Typically the signal peptide is clipped off by the host cell before the protein leaves the cell. Signal peptides can be found in variety of proteins in prokaryotes and eukaryotes.

One or multiple polynucleotides can encode an ADI protein described herein. Moreover, the polynucleotide sequence can be manipulated for various reasons. Examples include but are not limited to the incorporation of preferred codons to enhance the expression of the polynucleotide in various organisms (see generally Nakamura et al., Nuc. Acid. Res. 28:292, 2000).

Also included are expression vectors that comprise the polynucleotides, and host cells that comprise the polynucleotides and/or expression vectors. ADI proteins can be produced by expressing a DNA or RNA sequence encoding the polypeptide in a suitable host cell by well-known techniques. The term “host cell” is used to refer to a cell into which has been introduced, or which is capable of having introduced into it, a nucleic acid sequence encoding one or more of the polypeptides described herein, and which further expresses or is capable of expressing a polypeptide of interest, such as a polynucleotide encoding any herein described polypeptide. The term includes the progeny of the parent cell, whether or not the progeny are identical in morphology or in genetic make-up to the original parent, so long as the selected gene is present. Host cells may be chosen for certain characteristics, for instance, the expression of a formylglycine generating enzyme (FOE) to convert a cysteine or serine residue within a sulfatase motif into a formylglycine (FGly) residue, or the expression of aminoacyl tRNA synthetase(s) that can incorporate unnatural amino acids into the polypeptide, including unnatural amino acids with an azide side-chain, alkyne side-chain, or other desired side-chain, to facilitate chemical conjugation or modification.

In some instances, a polynucleotide or expression vector comprises additional non-coding sequences. For example, the “control elements” or “regulatory sequences” present in an expression vector are non-translated regions of the vector, including enhancers, promoters, 5′ and 3′ untranslated regions, which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used.

A variety of expression vector/host systems are known and may be utilized to contain and express polynucleotide sequences. These include, but are not limited to, microorganisms such as bacteria transformed with an expression vector, for example, a recombinant bacteriophage, plasmid, or cosmid DNA expression vector. Certain embodiments therefore include an expression vector, comprising a polynucleotide sequence that encodes a polypeptide described herein, for example, an ADI protein. Also included are host cells that comprise the polynucleotides and/or expression vectors.

Certain embodiments employ E. coli-based expression systems (see, e.g., Structural Genomics Consortium et al., Nature Methods. 5:135-146, 2008). These and related embodiments may optionally utilize ligation-independent cloning (LIC) to produce a suitable expression vector. In specific embodiments, protein expression may be controlled by a T7 RNA polymerase (e.g., pET vector series), or modified pET vectors with alternate promoters, including for example the TAC promoter. These and related embodiments may utilize the expression host strain BL21(DE3), a /∴DE3 lysogen of BL21 that supports T7-mediated expression and is deficient in Ion and ompT proteases for improved target protein stability. Also included are expression host strains carrying plasmids encoding tRNAs rarely used in E. coli, such as ROSETTA™ (DE3) and Rosetta 2 (DE3) strains. In some embodiments, other E. coli strains may be utilized, including other E. coli K-12 strains such as W3110 (F lambda⋅IN(rrnD-rrnE)1 rph-1), and UT5600 (F, araC14, leuB6(Am), secA206(aziR), lacY1, proC14, tsx67, .I\(ompTfepC)266, entA403, gInX44(AS), A, trpE38, rfbC1, rpsL109(strR), xylA5, rntl-1, thiE1), which can result in reduced levels of post-translational modifications during fermentation. Cell lysis and sample handling may also be improved using reagents sold under the trademarks BENZONASE® nuclease and BUGBUSTER® Protein Extraction Reagent. For cell culture, auto-inducing media can improve the efficiency of many expression systems, including high-throughput expression systems. Media of this type (e.g., OVERNIGHT EXPRESS™ Autoinduction System) gradually elicit protein expression through metabolic shift without the addition of artificial inducing agents such as IPTG.

Particular embodiments employ hexahistidine tags (such as those sold under the trademark HIS⋅TAG® fusions), followed by immobilized metal affinity chromatography (IMAC) purification, or related techniques. In certain aspects, however, clinical grade proteins can be isolated from E. coli inclusion bodies, without or without the use of affinity tags (see, e.g., Shimp et al., Protein Expr Purif. 50:58-67, 2006).

Also included are methods for recombinantly-producing an ADI protein, as described herein. In some embodiments, a polynucleotide encoding an ADI protein is introduced directly into a host cell, and the cell is incubated under conditions sufficient to induce expression of the encoded protein(s). The polypeptide sequences of this disclosure may be prepared using standard techniques well known to those of skill in the art in combination with the polypeptide and nucleic acid sequences provided herein.

Therefore, according to certain embodiments, there is provided a recombinant host cell that comprises a polynucleotide or a fusion polynucleotide which encodes an ADI protein described herein. Expression of an ADI protein in the host cell may be achieved by culturing under appropriate conditions recombinant host cells containing the polynucleotide. Following production by expression, the ADI protein may be isolated and/or purified using any suitable technique, and then used as desired. For instance, certain methods comprise: (a) expressing the ADI in a recombinant bacterial host cell, wherein the ADI is expressed in the host cell as insoluble and refoldable inclusion bodies; (b) removing the insoluble inclusion bodies from the host cell; (c) purifying the ADI from the insoluble inclusion bodies; (d) re-folding the ADI in a re-folding buffer; and (e) purifying the ADI from the re-folding buffer, thereby producing an isolated ADI (see the Examples).

In some embodiments, at least about 40, 50, 60, 70, 80, or 90% of the ADI is expressed in the bacterial host cell as insoluble and refoldable inclusion bodies. In particular embodiments, the host cell is E. coli.

The ADI proteins produced by a recombinant host cell can be purified and characterized according to a variety of techniques known in the art. Exemplary systems for performing protein purification and analyzing protein purity include fast protein liquid chromatography (FPLC) (e.g., AKTA and Bio-Rad FPLC systems), high-performance liquid chromatography (HPLC) (e.g., Beckman and Waters HPLC). Exemplary chemistries for purification include ion exchange chromatography (e.g., Q, S), size exclusion chromatography, salt gradients, affinity purification (e.g., Ni, Co, FLAG, maltose, glutathione, protein A/G), gel filtration, reverse-phase, ceramic HYPERD® ion exchange chromatography, and hydrophobic interaction columns (HIC), among others known in the art. See also the Examples.

Also included is assessing or measuring the ADI activity of the isolated ADI under physiological conditions, optionally of temperature and pH, wherein the isolated ADI has ADI activity under the physiological conditions. In some embodiments, the isolated ADI has at least about 50, 60, 70, 80, 90, 100, 110, or 120% of the ADI activity relative to an isolated ADI that consists of SEQ ID NO:1 (wild-type M. columbinum) under comparable physiological conditions.

Certain aspects further comprise preparing a composition that comprises the isolated ADI, for example, wherein the composition has a purity of at least about 80%, 85%, 90%, 95%, 98%, or 99% on a protein basis or a weight-weight basis, and wherein the composition is substantially aggregate-free and substantially endotoxin-free.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Modification of ADI from M. columbinum

Mutants of the ADI from M. columbinum were generated to identify those that increased expression of the protein as insoluble and refoldable inclusion bodies in E. coli. The ADI from M. columbinum (M.col ADI) is a 400 amino acid sequence with the amino terminal methionine removed during expression in E. coli. This protein has 35 lysine residues in its sequence. The attachment of polyethylene glycol (PEG) molecules to the surface of proteins often uses amine chemistry that links the PEG to lysines and the amino terminal amine. Thus, we initially sought to reduce the number of surface lysines in order to reduce the heterogeneity of PEGylation when these techniques are used. Further to that goal, the three-dimensional x-ray crystal structure of M.col ADI was solved to 2.4 angstroms and used to determine which of the 35 lysine residues were solvent-exposed and likely to be involved in PEGylation, and which residues were buried in the structure or involved in protein-protein interactions of the fully assembled homohexamer. Six of the lysines were identified as not being solvent-accessible.

The amino acid sequence of M.col ADI was then used to search the Genbank Database using the BLASTP program at blast.ncbi.nlm.nih.gov. Thirty-seven mycoplasma ADI enzymes were selected with amino acid identity ranging from 88%-53% and assembled into a multiple sequence alignment using AlignX from the VectorNTI Advance® 11.5.1 (Invitrogen) software suite. Each of the remaining 29 lysines in M.col ADI were compared to the 37 mycoplasma ADIs and any substitutions were listed.

Based on this analysis, a number of mutants were prepared. The sequence and activity information for each mutant discussed herein is summarized in Table A1.1-A1.2,Table A2 and Table A3.

Initially, all 29 lysines were mutated using the substitutions noted in the multiple sequence alignment. However, two lysines were absolutely conserved in all sequences and thus an alanine residue was chosen for the substitution at these positions. This mutant protein (McolM1) was expressed as insoluble inclusion bodies. The inclusion bodies were extracted, refolded, purified, and then tested for activity. McolM1 retained only ˜17% of the wild type enzyme activity. This low activity could result from improper folding of the mutant enzyme due to the large number of mutations introduced. Alternatively, certain of the lysine residues could be required for proper structure/function of the protein.

To identify lysine mutants that contribute to the insoluble phenotype and maintain most of the wild-type activity, the mutations were divided into two sets to create McolM2 and McolM3. These constructs were also insoluble and were refolded, purified and tested. McolM2 and McolM3 retained <80% of the wild type activity.

McolM2 was again split into McolM4 and McolM5, and McolM3 split into McolM6 and McolM7. McolM4 and McolM7 were expressed as soluble enzymes and retained >80% the wild type activity. McolM5 and McolM6 were insoluble and retained <80% activity. The mutants from McolM4 and McolM7 were then combined into McolM8, which expressed as a soluble enzyme and retained >80% of wild type activity.

The mutants from McolM5 and McolM6 were split and added to the McolM8 mutant set to create McolM9, McolM10, McolM11, and McolM12. All four mutants were insoluble, refolded, purified, and tested for activity with McolM9 and McolM10 being >80% active and McolM11 and McolM12<80% active. The McolM9 mutations demonstrated the first set of lysine mutations that allowed for high-level insoluble expression as an inclusion body and the ability to be refolded into an active enzyme with >80% of the wild type activity.

Because the only difference between McolM8 (soluble) and McolM9 (insoluble) was the addition of three mutants (K175R, K246E, K317R), each of these three mutations in McolM9 was converted back to lysine, one at a time, to create each pairwise combination and each single mutant (see McolM29-McolM34). All six mutants were expressed as insoluble inclusion bodies and refolded with most having activity >80%. McolM33 was chosen as a backbone sequence and lysine mutants were then selectively mutated back to lysine starting at the extreme carboxy and amino regions of the protein. McolM59-McolM63, respectively, had 2, 3, 4, 5, and 6 lysines mutants replaced with wild-type lysine. McolM59 and McolM60 were insoluble, refolded, purified, tested, and found to have >80% wild type activity. McolM61 had an intermediate phenotype with only ˜50% of protein expressed in an insoluble inclusion body. The insoluble portion was refolded, purified, tested, and found to have >80% of the wild type activity. McolM62 and McolM63 were soluble and were not tested.

Other factors that can affect solubility were also tested. For instance, codon usage for back translation from an amino acid sequence to a DNA sequence for protein expression can affect the level of protein expression in E. coli. In general, the more a protein sequence utilizes the preferred codons of E. coli, the higher the protein expression (see, e.g., Gouy and Gautier, Nucleic Acids Res. 10:7055-74, 1982). Tests were thus performed to determine whether codon usage could affect the solubility of M. col ADI expressed in E. coli, by varying the codon preference as measured by the codon adaptation index (CAI) (see, e.g., Sharp et al., Nucleic Acids Res. 15:1281-1295, 1987). The CAI is expressed as a number from 1.0 to <1 with 1.0 representing a perfect match for codon preference of the organism being used as an expression host and the gene of interest. Unexpectedly, it was found that a single change in McolM8 of one lysine codon (K317) from the preferred AAA to the non-preferred AAG rendered the resulting construct (McolM46) insoluble and refoldable.

Pegylation-related mutation strategies were also tested. For example, further to the random PEGylation on amines strategy, substitution of solvent accessible residues with cysteine can offer a mechanism for sight specific PEGylation on a molecule (see, e.g., Dozier and Distefano, Int J Mol Sci. 16:25831-25864, 2015). Because of the homohexameric structure of M. col ADI, a single cysteine mutation allows for up to six PEG molecules to be attached to the functional enzyme. A number of solvent-accessible residues were mutated with cysteine on the wild type M. col ADI sequence, the most effective being K192C and K287C, which had little if any impact on the structure/function of ADI. These mutations were made in the McolM8 soluble background, both as single mutants and as a double mutant (respectively, McolM35, McolM36, and McolM39). All three were expressed as insoluble/refoldable inclusion bodies and were capable of being pegylated with 20 kDa maleimide PEG, which specifically modifies solvent accessible cysteine residues. These results show that the current set of lysine mutations in the McolM8 soluble construct could be made insoluble/refoldable by merely changing the lysine-substituted amino acid(s) to cysteine, rather than adding more lysine mutations.

Overall, these results show that substitutions and/or codon usage changes to selected lysine residues can be employed to produce modified ADIs from M. columbinum that not only form insoluble and refoldable inclusion bodies in E co/i, but also retain significant ADI activity under physiological conditions.

### Materials and Methods:

Molecular Biology: Unless otherwise indicated, buffers salts and general reagents were purchased from Sigma-Aldrich. The amino acid sequence for Mycoplasma columbinum arginine deiminase (Sequence ID EGV00288 and mutant constructs of this sequence) was back translated to a DNA sequence using Vector NTI advance 11.5.1 software (Invitrogen) and the Escherichia coli standard codon preference table. A unique NdeI restriction site was included at the 5′ terminus encoding the starting methionine and a unique XhoI site was added to the 3′ terminus forming a fusion with the vector encoded hexahistidine tag.

This DNA sequence was synthesized by Invitrogen and supplied in their standard vector. The vector was restricted with NdeI and XhoI enzymes (New England Biolabs) and visualized on a 1% agarose E-gel (Invitrogen). The 1.2 kb NdeI-XhoI DNA fragment containing the M. columbinum arginine deiminase (M.col ADI) gene was excised from the gel under blue light Safe-Imager 2.0 (Invitrogen) and purified with a QIAquick gel extraction kit (Qiagen). The XhoI-NdeI digested expression vector pET21a (Novagen) was prepared in the same manor. Vector and insert DNA were ligated with a Quick Ligation Kit (New England Biolabs) and transformed into chemically competent Mach1-T1 cells (Invitrogen) and selected on LB agar with 100 ug/ml of Ampicillin (Teknova). Single colonies were inoculated into 5 mls of LB media supplemented with 100 ug/ml of Ampicillin (GIBCO) and grown to an OD600 of ˜1.

One ml aliquots with 20% glycerol, final concentration, were flash frozen in liquid nitrogen for storage and later use. Plasmid DNA was made from the remaining culture using QIAprep Spin Miniprep Kit (Qiagen). The insert sequence was verified by automated dideoxy chain termination sequencing (Retrogen, San Diego CA). Verified sequences were then transformed into the expression host BL21(DE3) (Invitrogen) and frozen stocks made as mentioned above. Site directed mutagenesis of mutant residues back to their wild type lysine was accomplished using QuickChange II XL Site-Directed Mutagenesis Kit (Agilent Technologies, San Diego CA) following the manufactures protocol.

All mutations were sequence-confirmed over the entire length of the gene sequence to rule out any secondary defects that could have been introduced by the mutagenesis procedure. Select insoluble mutants of M. col ADI were also mutated to remove the carboxy terminal hexahistidine tag by inserting a stop codon (TAG) just before the XhoI restriction site using site directed mutagenesis to confirm the insoluble/refoldable active phenotype.

Expression and Purification: The expression of the Mycoplasma columbinum ADI wild-type and mutant proteins was essentially the same for each construct. In general, frozen glycerol stocks of the expression host were inoculated into 5 ml of LB media with 100 ug/ml ampicillin and allowed to grow overnight at 37° C. A 1:1000 dilution was made from this starter culture into 1 liter of Terrific Broth auto induction media (see, e.g., Studier, Protein Expression and Purif. 41:207-234, 2005), with 100 pg/ml ampicillin, in baffled Erlenmeyer flasks. Cells were grown at 37° C. overnight at 250 rpm in a shaking incubator. Cell pellets were harvested by centrifugation at 10,000 rpm for 20 minutes and then stored at −80° C. for future purification.

Mycoplasma columbinum ADI wild-type: Cell pellets harboring expressed ADI were re-suspended in 20 mM NaPO₄ pH 8.5 and 20 mM imidazole at a ratio of 5 mls of buffer per gram of cell paste. The cells were then lysed using an M110L microfluidizer (Microfluidics, Westwood MA). Insoluble material was removed by centrifugation at 10,000 rpm. The supernatant was loaded onto a 50 ml NiNTA Superflow (Qiagen) column equilibrated with 20 mM NaPO₄ pH 8.5, 20 mM imidazole, using an AKTA FPLC (GE Amersham Pharmacia). The column was washed to baseline with 20 mM NaPO₄ pH 8.5, 20 mM imidazole and then the bound protein was eluted with a step gradient of 20 mM NaPO₄ pH 8.5 500 mM imidazole.

The eluted protein was then bound to a 50 ml Q-Sepharose Fast Flow (GE Amersham Pharmacia) column and washed to baseline with 20 mM NaPO₄ pH 8.5. The bound protein was eluted with a linear gradient of 20 mM NaPO₄ pH 8.5 to 20 mM NaPO₄ pH 8.5, 1M NaCl over 10 column volumes. Peak fractions were pooled, made 1 M with ammonium sulfate, and then loaded onto a 50 ml Phenyl Sepharose High Performance (GE Amersham Pharmacia) column. The column was washed to baseline with 20 mM NaPO₄ pH 8.5, 1 M ammonium sulfate then the bound protein was eluted with a linear gradient of 20 mM NaPO₄ pH 8.5, 1 M ammonium sulfate to 20 mM NaPO₄ pH 8.5 over 10 column volumes.

The peak fractions were pooled, concentrated to ˜20 ml or less in an Amicon stir cell concentrator with 10 kDa MWCO Ultracel® membrane (EMD Millipore, Billerica MA), sterile filtered through a 25 mm 0.2 micron Supor® syringe filter (Pall Life Sciences), then loaded onto a 500 ml Superdex 200 size exclusion column (GE Amersham Pharmacia). The column was developed at 1 ml/min in 10 mM HEPES pH 7.5 150 mM NaCl buffer. Peak fractions were pooled and concentrated to 1 mg/ml or more and stored at −80° C.

Mycoplasma columbinum ADI insoluble mutants: Insoluble mutants were purified in the same way as wild type protein with the following differences. After cell lysis in 20 mM NaPO₄ pH 8.5 buffer, the pellet formed from the 10,000 rpm centrifugation were suspended in several hundred mls of 20 mM NaPO₄ pH 8.5 wash buffer using a hand-held homogenizer (Power Gen 125, Fisher Scientific, Hampton NH) and centrifuged again. This wash step was repeated several times until a clear supernatant was formed.

The insoluble inclusion bodies in the pellet can then be refolded using the methods described in the literature (see, e.g., U.S. Pat. No. 6,132,713; Misawa et al., J Biotechnol. 36:145-55, 1994; and Noh et al., Molecules and Cells. 13:137-143, 2002). The refolded active ADI was then purified from the refold buffer starting with the Q-Sepharose Fast Flow step and following all subsequent steps as was done with the wild type protein.

Protein purity assessment: Typically, protein purity is assessed during the purification process by the chromatographic behavior of the protein followed by polyacrylamide gel electrophoresis (PAGE) followed by coomassie blue protein staining and gel densitometry analysis. Also, as a final test after protein PEGylation, reverse phase liquid chromatography (RPLC) is used to determine average peg number and purity. Proteins purified using the methods described have purity values >95%.

Activity Assay: ADI catalyzes the conversion of L-arginine to L-citrulline and ammonia. The amount of L-citrulline can be detected by a colorimetric endpoint assay (Knipp and Vasak, Anal Biochem. 286:257-64, 2000) and compared to a standard curve of known amounts of L-citrulline in order to calculate the specific activity of ADI expressed as IU/mg of protein. One IU of enzyme activity was defined as the amount of enzyme that produces 1 μmol of citrulline per minute at the pH and temperature being tested. Standard assay conditions were performed at 37° C. in Physiological HEPES Buffer (PHB), 50 mM HEPES, 160 mM NaCl pH 7.4 (Clin Chem Lab Med. 37:563-71, 1999) plus 0.1% BSA. All samples and standards were run in duplicate or triplicate where conditions permitted.

Km and Kcat values were determined by using a variation of the activity assay described above. As with the activity assay (or unless otherwise indicated), all reactions were run at 37° C. in PHB plus 0.1% BSA. Enzyme concentration, reaction time, and substrate concentration range were adjusted for each of the ADI constructs to account for their differences in activity. In general, 2 nM enzyme, 5-minute reaction time, and a 0-160 μM arginine were used as starting conditions. When optimizing the conditions, particular attention was paid towards the amount of substrate consumed as a percentage of total substrate added to the reaction. Typically, the lower limit of detection was about 1 μM of citrulline with the lower limit of quantitation being about 2 μM. A citrulline standard curve was run on every plate and used to quantify the citrulline produced by the enzymatic reaction.

Calculations: The citrulline concentration (μM) produced in each reaction well was calculated and averaged using the citrulline standard curve. The velocity of each reaction was then calculated in μM/min/50 nM ADI. Specific activity (IU/mg or μmols product/min/mg ADI) was calculated by multiplying this value by the “IU” factor (IU factor was calculated from the molecular weight of the ADI and the reaction volume).

### Example 2

### Modification of ADI from M. columbinum

Additional mutants of the ADI from M. columbinum were generated to identify those that increased expression of the protein as insoluble and refoldable inclusion bodies in E. coli. The sequence and activity information for each mutant discussed herein is summarized in Table A1.1-A1.2, Table A2 and Table A3. The additional mutants of the ADI were tested using the methods described above in Example 2.

Mutant ADIs M1-M56, as described above identified a subset of lysine mutations that confer the original desired phenotype, e.g., insoluble, refoldable protein with retained ADI activity. M34 was found to have the most desirable phenotype. Data generated from studies of mutant ADIs M59-M86 led to the identification of a subset of substitutions that were found to be important for the desired phenotype. These mutants were identified as K90T, K108R, K192V, K216N, K240V, K287Q and K295A. The first six substitutions are found to be important while the last one, K295A, is found to be of less importance. Accordingly, these seven mutants are found to be important for conferring the desired phenotype and are represented by M86.

Additional lysine codon changes were also included in the studies and found to be of varied importance. M94-M184 mutants were studied which changed each of the identified seven mutants to other amino acids using M34 as the background for these changes. Of the twenty amino acids available for substitution at each of the lysine positions, thirteen are left if you ignore the use of lysine (wild type) the original mutation (varies per site), proline (causes structural changes), cysteine (can form disulfides with other molecules, cysteine which can be used for site specific pegylation if desired, M35, M36 and M39.), bulky hydrophobic residues tryptophan, phenylalanine and tyrosine (Lysine is on the surface of the protein and large hydrophobic side chains are unfavorable on the surface of a protein and could cause aggregation). This leaves 13 other amino acids that can be substituted at each of the seven lysine positions, M94-M184. Each was assessed for insoluble expression of ADI from M. columbinum and a select few from each position were refolded, purified and activity compared to the wild type enzyme.

Seven changes were selected and incorporated into a single mutant, M188. This mutant did not have proper enzyme activity, so each position was converted back to wild type lysines, represented as M189-M195. No single change resulted in the desired phenotype, so each individual mutation was added to find the minimum necessary for the desired phenotype. The K246E substitution was added due to its ability to increase the activity of the enzyme by ˜5%. M196 to M122 show the results of the sequential addition. M214, has the desired phenotype and only requires three mutations in addition to the K246E substitution, i.e., K90V, K287A and K2951. M216 and M218 also have the desired phenotype but have four mutations each, in addition to the K246E substitution, with M218 sharing the same three from M214 and M216 sharing only 2 of the three from M214. While the data presented herein demonstrates a number of active mutant ADI proteins with desirable characteristics, it is likely that additional combinations of mutations will result in the same phenotypes and such combinations may be identified using the methods disclosed herein.

1. An isolated arginine deiminase (ADI), comprising, consisting, or consisting essentially of an amino acid sequence that is at least 90, 95, 96, 97, 98, 99, or 100% identical to an amino sequence selected from Table A1.1 and A1.2, excluding SEQ ID NO:1.

2. The isolated ADI of claim 1, wherein the isolated ADI is recombinantly expressed (or expressible) in a bacterial host cell, optionally E. coli, as insoluble and refoldable inclusion bodies.

3. The isolated ADI of claim 2, wherein at least about 10-100% or at least about 10, 20, 30, 40, 50, 60, 70, 80, 90, or 100% of the ADI is recombinantly expressed (or expressible) in the bacterial host cell as insoluble and refoldable inclusion bodies.

4. The isolated ADI of claim 1, wherein the isolated ADI has ADI activity under physiological conditions, optionally of temperature, salinity, and pH.

5. The isolated ADI of claim 4, which has at least about 50, 60, 70, 80, 90, 100, 110, or 120% of the ADI activity relative to an isolated ADI that consists of SEQ ID NO:1 (wild-type M. columbinum) under comparable physiological conditions.

6. The isolated ADI of claim 1, comprising, consisting, or consisting essentially of an amino acid sequence that is at least 90, 95, 96, 97, 98, 99, or 100% identical to an amino acid sequence selected from SEQ ID NOs:2-178, which retains one or more lysine substitutions selected from K2G, K13E, K63N, K82S, K90T, K90V, K101D, K106L, K108R, K108A, K131R, K170R, K175R, K192V, K192C, K216N, K216V, K229L, K237N, K238N, K240V, K243T, K246E, K248R, K249R, K273R, K275A, K287Q, K287C, K287A, K295A, K2951, K304L, K317R, K326A, and K400A
(relative to SEQ ID NO: 1).

7. The isolated ADI of claim 6, which retains 1, 2, 3, 4, 5, 6, 7, 8, 9, 10, 11, 12, 13, 14, 15, 16, 17, 18, 19, 20, 21, 22, 23, 24, 25, 26, 27, 28, or 29 of the lysine substitutions selected from K2G, K13E, K63N, K82S, K90T, K90V, K101D, K106L, K108R, K108A, K131R, K170R, K175R, K192V, K192C, K216N, K216V, K229L, K237N, K238N, K240V, K243T, K246E, K248R, K249R, K273R, K275A, K287Q, K287C, K287A, K295A, K2951, K304L, K317R, K326A, and K400A (relative to SEQ I NO: 1), optionally all or a portion of the lysine substitutions indicated in Table A2 and A3 for the selected sequence.

8. The isolated ADI of claim 1, wherein the isolated ADI is covalently bonded via a linker to at least one PEG molecule.

9. The isolated ADI of claim 8, wherein the isolated ADI is covalently bonded to about 1 to about 10 PEG molecules.

10. The isolated ADI of claim 8, wherein the isolated ADI is covalently bonded to about 2 to about 8 PEG molecules.

11. The isolated ADI of claim 8, wherein the PEG molecules are straight chain or branch chain PEG molecules.

12. The isolated ADI of claim 8, wherein the PEG has a total weight average molecular weight of from about 1,000 to about 40,000, optionally from about 2,000 to about 20,000, optionally from about 2,000 to about 10,000, optionally about 5,000.

13. The isolated ADI of claim 8, wherein the linker is a succinyl group, an amide group, an imide group, a carbamate group, an ester group, an epoxy group, a carboxyl group, a hydroxyl group, a carbohydrate, a tyrosine group, a cysteine group, a histidine group, a methylene group, or any combinations thereof.

14. The isolated arginine deiminase of claim 13, wherein the source of the succinyl group is a succinimidyl carboxymethyl ester (SCM) or N-hydroxy succinimide (NHS).

15. A therapeutic composition, comprising the isolated arginine deiminase (ADI) of claim 1, and a pharmaceutically-acceptable carrier.

16. The therapeutic composition of claim 15, wherein the composition has a purity of at least about 80%, 85%, 90%, 95%, 98%, or 99% on a protein basis or a weight-weight basis and is substantially aggregate-free.

17. The therapeutic composition of claim 15, which is substantially endotoxin-free.

18. A method of treating, ameliorating the symptoms of, or inhibiting the progression of, a cancer in a subject in need thereof, comprising administering to the subject the therapeutic composition of claim 15.

19. The method of claim 18, wherein the cancer is selected from one or more of hepatocellular carcinoma (HCC), melanoma, metastatic melanoma, pancreatic cancer, prostate cancer, small cell lung cancer, mesothelioma, lymphocytic leukemia, chronic myelogenous leukemia, lymphoma, hepatoma, sarcoma, leukemia, acute myeloid leukemia, relapsed acute myeloid leukemia, B-cell malignancy, breast cancer, ovarian cancer, colorectal cancer, gastric cancer, glioma (e.g., astrocytoma, oligodendroglioma, ependymoma, or a choroid plexus papilloma), glioblastoma multiforme (e.g., giant cell gliobastoma or a gliosarcoma), meningioma, pituitary adenoma, vestibular schwannoma, primary CNS lymphoma, primitive neuroectodermal tumor (medulloblastoma), non-small cell lung cancer (NSCLC), kidney cancer, bladder cancer, uterine cancer, esophageal cancer, brain cancer, head and neck cancers, cervical cancer, testicular cancer, and stomach cancer.

20. The method of claim 18, wherein the cancer exhibits reduced expression of argininosuccinate synthetase-1.

21. A polynucleotide encoding the isolated arginine deiminase (ADI) of claim 1, or a vector comprising the polynucleotide.

22. The polynucleotide or vector of claim 21, comprising at least one AAG codon that encodes a lysine residue, optionally the K317 residue.

23. The polynucleotide or vector of claim 22, comprising at least about 1, 2, 3, 4, 5, 6, 7, 8, 9, 10, 11, 12, 13, 14, 15, 16, 17, 18, 19, or 20 AAG codons that encode a lysine residue.

24. A recombinant bacterial host cell, optionally E. coli, comprising a polynucleotide or vector of claim 21.

25. A method for recombinantly-producing an isolated arginine deiminase (ADI), comprising
(a) expressing the ADI in a recombinant bacterial host cell of claim 24, wherein the ADI is expressed in the host cell as insoluble and refoldable inclusion bodies;
(b) removing the insoluble inclusion bodies from the host cell;
(c) purifying the ADI from the insoluble inclusion bodies;
(d) re-folding the ADI in a re-folding buffer; and
(e) purifying the ADI from the re-folding buffer, thereby producing an isolated ADI.

26. The method of claim 25, wherein at least about 10-100% or at least about 10, 20, 30, 40, 50, 60, 70, 80, 90, or 100% of the ADI is expressed in the bacterial host cell as insoluble and refoldable inclusion bodies.

27. The method of claim 26, further comprising measuring the ADI activity of the isolated ADI under physiological conditions, optionally of temperature, salinity, and pH, wherein the isolated ADI has ADI activity under the physiological conditions.

28. The method of claim 27, wherein the isolated ADI has at least about 50, 60, 70, 80, 90, 100, 110, or 120% of the ADI activity relative to an isolated ADI that consists of SEQ ID NO:1 (wild-type M. columbinum) under comparable physiological conditions.

29. The method of claim 25, further comprising preparing a therapeutic composition that comprises the isolated ADI, wherein the composition has a purity of at least about 80%, 85%, 90%, 95%, 98%, or 99% on a protein basis or a weight-weight basis, and wherein the composition is substantially aggregate-free and substantially endotoxin-free.


SEQUENCE LISTING (178 sequences)

SEQ ID NO: 1 - protein, 400 aa, Mycoplasma columbinum
SKINVYSEIGELKEVLVHTPGDEIRRISPSRLDELLFSAILEPNEAIKEHKGFLKILQDKGIKVIQLSDLVAETYTYHATQKEREAFIEKWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNKDTLVIGVSERTNKEAILTIAKKIKNNKEAKFKKIVAINVPPMPNLMHLDTWLTMVDKDKFLYSPNMLSVLKVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 2 - protein, 400 aa, synthetic construct
  REGION 1..400: note = Synthetic: McolM1 Refold
SGINVYSEIGELEEVLVHTPGDEIRRISPSRLDELLFSAILEPNEAIKEHKGFLKILQDKGINVIQLSDLVAETYTYHATQSEREAFIETWLDEAEPALTDDLRALVRSYVLSKEGTPVAMVRTMMAGVSRQELNVESETELVVDPMPNLYFTRDPFASAGNGISLNNMRYVTRRRETIFAEFIFATHPDYVTTPHWFDRLDEGNIEGGDVFIYNNDTLVIGVSERTNLEAILTIANNIVNNTEAEFRRIVAINVPPMPNLMHLDTWLTMVDRDAFLYSPNMLSVLQVWEIDLSAEIEMVETNLPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 3 - protein, 400 aa, synthetic construct
  REGION 1..400: note = Synthetic: McolM2 Refold
SGINVYSEIGELKEVLVHTPGDEIRRISPSRLDELLFSAILEPNEAIKEHKGFLKILQDKGINVIQLSDLVAETYTYHATQKEREAFIETWLDEAEPALTKDLRALVKSYVLSKEGTPVAMVRTMMAGVSRQELNVESETELVVDPMPNLYFTRDPFASAGNGISLNNMKYVTRRRETIFAEFIFATHPDYKTTPHWFDRLDEGNIEGGDVFIYNNDTLVIGVSERTNKEAILTIANKIVNNKEAEFKRIVAINVPPMPNLMHLDTWLTMVDKDAFLYSPNMLSVLKVWEIDLSAEIEMVETNKPLADVLESIIGVRPVLIPIAGKGATQLDIDIETHFDGTNYLTIAPGVVVGYSRNIKTEAALRAAGVTVLSFEGNQLSLGMGSARCMSMPLVREDVA

SEQ ID NO: 4 - protein, 400 aa, synthetic construct
  REGION 1..400: note = Synthetic: McolM3 Refold
SGINVYSEIGELEEVLVHTPGDEIRRISPSRLDELLFSAILEPNEAIKEHKGFLKILQDKGIKVIQLSDLVAETYTYHATQSEREAFIEKWLDEAEPALTDDLRAKVRSYVLSKEGTPVAMVRTMMAGVSKQELNVESETELVVDPMPNLYFTRDPFASAGNGISLNNMRYVTRKRETIFAEFIFATHPDYVTTPHWFDRLDEGNIEGGDVFIYNKDTLVIGVSERTNLEAILTIAKNIKNNTEAKFRKIVAINVPPMPNLMHLDTWLTMVDRDKFLYSPNMLSVLQVWEIDLSKEIEMVETNLPLADVLESIIGVKPVLIPIAGAGATQLDIDIETHFDGTNYLTIAPGVVVGYSRNIKTEAALRAAGVTVLSFEGNQLSLGMGSARCMSMPLVREDVK

SEQ ID NO: 5 - protein, 400 aa, synthetic construct
  REGION 1..400: note = Synthetic: McolM4 Soluble
SGINVYSEIGELKEVLVHTPGDEIRRISPSRLDELLFSAILEPNEAIKEHKGFLKILQDKGIKVIQLSDLVAETYTYHATQKEREAFIETWLDEAEPALTKDLRAKVKSYVLSKEGTPVAMVRTMMAGVSRQELNVESETELVVDPMPNLYFTRDPFASAGNGISLNNMKYVTRKRETIFAEFIFATHPDYKTTPHWFDRLDEGNIEGGDVFIYNNDTLVIGVSERTNKEAILTIAKKIVNNKEAKFKRIVAINVPPMPNLMHLDTWLTMVDKDKFLYSPNMLSVLKVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVA

SEQ ID NO: 6 - protein, 400 aa, synthetic construct
  REGION 1..400: note = Synthetic: McolM5 Refold
SGINVYSEIGELKEVLVHTPGDEIRRISPSRLDELLFSAILEPNEAIKEHKGFLKILQDKGINVIQLSDLVAETYTYHATQKEREAFIEKWLDEAEPALTKDLRALVKSYVLSKEGTPVAMVRTMMAGVSKQELNVESETELVVDPMPNLYFTRDPFASAGNGISLNNMKYVTRRRETIFAEFIFATHPDYKTTPHWFDRLDEGNIEGGDVFIYNKDTLVIGVSERTNKEAILTIANKIKNNKEAEFKKIVAINVPPMPNLMHLDTWLTMVDKDAFLYSPNMLSVLKVWEIDLSKEIEMVETNKPLADVLESIIGVRPVLIPIAGKGATQLDIDIETHFDGTNYLTIAPGVVVGYSRNIKTEAALRAAGVTVLSFEGNQLSLGMGSARCMSMPLVREDVK

SEQ ID NO: 7 - protein, 400 aa, synthetic construct
  REGION 1..400: note = Synthetic: McolM6 Refold
SGINVYSEIGELEEVLVHTPGDEIRRISPSRLDELLFSAILEPNEAIKEHKGFLKILQDKGIKVIQLSDLVAETYTYHATQKEREAFIEKWLDEAEPALTDDLRAKVKSYVLSKEGTPVAMVRTMMAGVSKQELNVESETELVVDPMPNLYFTRDPFASAGNGISLNNMRYVTRKRETIFAEFIFATHPDYKTTPHWFDRLDEGNIEGGDVFIYNKDTLVIGVSERTNLEAILTIAKKIKNNTEAKFKKIVAINVPPMPNLMHLDTWLTMVDRDKFLYSPNMLSVLKVWEIDLSKEIEMVETNLPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 8 - protein, 400 aa, synthetic construct
  REGION 1..400: note = Synthetic: McolM7 Soluble
SGINVYSEIGELKEVLVHTPGDEIRRISPSRLDELLFSAILEPNEAIKEHKGFLKILQDKGIKVIQLSDLVAETYTYHATQSEREAFIEKWLDEAEPALTKDLRAKVRSYVLSKEGTPVAMVRTMMAGVSKQELNVESETELVVDPMPNLYFTRDPFASAGNGISLNNMKYVTRKRETIFAEFIFATHPDYVTTPHWFDRLDEGNIEGGDVFIYNKDTLVIGVSERTNKEAILTIAKNIKNNKEAKFRKIVAINVPPMPNLMHLDTWLTMVDKDKFLYSPNMLSVLQVWEIDLSKEIEMVETNKPLADVLESIIGVKPVLIPIAGAGATQLDIDIETHFDGTNYLTIAPGVVVGYSRNIKTEAALRAAGVTVLSFEGNQLSLGMGSARCMSMPLVREDVK

SEQ ID NO: 9 - protein, 400 aa, synthetic construct
  REGION 1..400: note = Synthetic: McolM8 Soluble
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVKPVLIPIAGAGATQLDIDIETHFDGTNYLTIAPGVVVGYSRNIKTEAALRAAGVTVLSFEGNQLSLGMGSARCMSMPLVREDVA

SEQ ID NO: 10 - protein, 400 aa, synthetic construct
  REGION 1..400: note = Synthetic: McolM9 Refold
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRRRETIFAEFIFATHPDYVTTPHWFDRLDEGNIEGGDVFIYNNDTLVIGVSERTNKEAILTIAKNIVNNKEAEFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 11 - protein, 400 aa, synthetic construct
  REGION 1..400: note = Synthetic: McolM10 Refold
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRALVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIANNIVNNKEAKFRRIVAINVPPMPNLMHLDTWLTMVDKDAFLYSPNMLSVLQVWEIDLSAEIEMVETNKPLADVLESIIGVKPVLIPIAGAGATQLDIDIETHFDGTNYLTIAPGVVVGYSRNIKTEAALRAAGVTVLSFEGNQLSLGMGSARCMSMPLVREDVA

SEQ ID NO: 12 - protein, 400 aa, synthetic construct
  REGION 1..400: note = Synthetic: McolM11 Refold
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMRYVTRKRETIFAEFIFATHPDYVTTPHWFDRLDEGNIEGGDVFIYNNDTLVIGVSERTNKEAILTIAKNIVNNTEAKFRRIVAINVPPMPNLMHLDTWLTMVDKDKFLYSPNMLSVLQVWEIDLSAEIEMVETNLPLADVLESIIGVKPVLIPIAGAGATQLDIDIETHFDGTNYLTIAPGVVVGYSRNIKTEAALRAAGVTVLSFEGNQLSLGMGSARCMSMPLVREDVA

SEQ ID NO: 13 - protein, 400 aa, synthetic construct
  REGION 1..400: note = Synthetic: McolM12 Refold
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTDDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNLEAILTIAKNIVNNKEAKFRRIVAINVPPMPNLMHLDTWLTMVDRDKFLYSPNMLSVLQVWEIDLSAEIEMVETNKPLADVLESIIGVKPVLIPIAGAGATQLDIDIETHFDGTNYLTIAPGVVVGYSRNIKTEAALRAAGVTVLSFEGNQLSLGMGSARCMSMPLVREDVA

SEQ ID NO: 14 - protein, 400 aa, synthetic construct
  REGION 1..400: note = Synthetic: McolM29 Refold
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAEFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 15 - protein, 400 aa, synthetic construct
  REGION 1..400: note = Synthetic: McolM30 Refold
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRRRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 16 - protein, 400 aa, synthetic construct
  REGION 1..400: note = Synthetic: McolM31 Refold
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRRRETIFAEFIFATHPDYVTTPHWFDRLDEGNIEGGDVFIYNNDTLVIGVSERTNKEAILTIAKNIVNNKEAEFRRIVAINVPPMPNLMHLDTWLTMVDKDKFLYSPNMLSVLQVWEIDLSAEIEMVETNKPLADVLESIIGVKPVLIPIAGAGATQLDIDIETHFDGTNYLTIAPGVVVGYSRNIKTEAALRAAGVTVLSFEGNQLSLGMGSARCMSMPLVREDVA

SEQ ID NO: 17 - protein, 400 aa, synthetic construct
  REGION 1..400: note = Synthetic: McolM32 Refold
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRRRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVKPVLIPIAGAGATQLDIDIETHFDGTNYLTIAPGVVVGYSRNIKTEAALRAAGVTVLSFEGNQLSLGMGSARCMSMPLVREDVA

SEQ ID NO: 18 - protein, 400 aa, synthetic construct
  REGION 1..400: note = Synthetic: McolM33 Refold
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAEFRRIVAINVPPMPNLMHLDTWLTMVDKDKFLYSPNMLSVLQVWEIDLSAEIEMVETNKPLADVLESIIGVKPVLIPIAGAGATQLDIDIETHFDGTNYLTIAPGVVVGYSRNIKTEAALRAAGVTVLSFEGNQLSLGMGSARCMSMPLVREDVA

SEQ ID NO: 19 - protein, 400 aa, synthetic construct
  REGION 1..400: note = Synthetic: McolM34 Refold
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 20 - protein, 400 aa, synthetic construct
  REGION 1..400: note = Synthetic: McolM35 Refold
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYCTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVKPVLIPIAGAGATQLDIDIETHFDGTNYLTIAPGVVVGYSRNIKTEAALRAAGVTVLSFEGNQLSLGMGSARCMSMPLVREDVA

SEQ ID NO: 21 - protein, 400 aa, synthetic construct
  REGION 1..400: note = Synthetic: McolM36 Refold
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLCVWEIDLSAEIEMVETNKPLADVLESIIGVKPVLIPIAGAGATQLDIDIETHFDGTNYLTIAPGVVVGYSRNIKTEAALRAAGVTVLSFEGNQLSLGMGSARCMSMPLVREDVA

SEQ ID NO: 22 - protein, 400 aa, synthetic construct
  REGION 1..400: note = Synthetic: McolM39 Refold
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYCTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLCVWEIDLSAEIEMVETNKPLADVLESIIGVKPVLIPIAGAGATQLDIDIETHFDGTNYLTIAPGVVVGYSRNIKTEAALRAAGVTVLSFEGNQLSLGMGSARCMSMPLVREDVA

SEQ ID NO: 23 - protein, 400 aa, synthetic construct; identical to SEQ ID NO: 9
  REGION 1..400: note = Synthetic: McolM46 Refold

SEQ ID NO: 24 - protein, 400 aa, synthetic construct
  REGION 1..400: note = Synthetic: McolM56 Soluble
SRINVYSEIGELKEVLVHTPGDEIRRISPSRLDELLFSAILEPNEAIKEHKGFLKILQDKGIKVIQLSDLVAETYTYHATQREREAFIERWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYRTTPHWFDRLDEGNIEGGDVFIYNRDTLVIGVSERTNKEAILTIAKRIRNNKEAEFRRIVAINVPPMPNLMHLDTWLTMVDKDKFLYSPNMLSVLRVWEIDLSREIEMVETNKPLADVLESIIGVKPVLIPIAGRGATQLDIDIETHFDGTNYLTIAPGVVVGYSRNIKTEAALRAAGVTVLSFEGNQLSLGMGSARCMSMPLVREDVR

SEQ ID NO: 25 - protein, 400 aa, synthetic construct
  REGION 1..400: note = Synthetic: McolM59 Refold
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAEFRRIVAINVPPMPNLMHLDTWLTMVDKDKFLYSPNMLSVLQVWEIDLSAEIEMVETNKPLADVLESIIGVKPVLIPIAGAGATQLDIDIETHFDGTNYLTIAPGVVVGYSRNIKTEAALRAAGVTVLSFEGNQLSLGMGSARCMSMPLVREDVK

SEQ ID NO: 26 - protein, 400 aa, synthetic construct
  REGION 1..400: note = Synthetic: McolM60 Refold
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAEFRRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 27 - protein, 400 aa, synthetic construct
  REGION 1..400: note = Synthetic: McolM61 50/50
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAEFRRIVAINVPPMPNLMHLDTWLTMVDKDKFLYSPNMLSVLQVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 28 - protein, 400 aa, synthetic construct
  REGION 1..400: note = Synthetic: McolM62 Soluble
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAEFRRIVAINVPPMPNLMHLDTWLTMVDKDKFLYSPNMLSVLKVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 29 - protein, 400 aa, synthetic construct
  REGION 1..400: note = Synthetic: McolM63 Soluble
SKINVYSEIGELKEVLVHTPGDEIRRISPSRLDELLFSAILEPNEAIKEHKGFLKILQDKGIKVIQLSDLVAETYTYHATQKEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAEFRRIVAINVPPMPNLMHLDTWLTMVDKDKFLYSPNMLSVLKVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 30 - protein, 400 aa, synthetic construct
  REGION 1..400: note = Synthetic: McolM64
SKINVYSEIGELKEVLVHTPGDEIRRISPSRLDELLFSAILEPNEAIKEHKGFLKILQDKGIKVIQLSDLVAETYTYHATQKEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAEFRRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 31 - protein, 400 aa, synthetic construct
  REGION 1..400: note = Synthetic: McolM65
SKINVYSEIGELKEVLVHTPGDEIRRISPSRLDELLFSAILEPNEAIKEHKGFLKILQDKGIKVIQLSDLVAETYTYHATQSEREAFIEKWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAEFRRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 32 - protein, 400 aa, synthetic construct
  REGION 1..400: note = Synthetic: McolM66
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVKSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAEFRRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 33 - protein, 400 aa, synthetic construct
  REGION 1..400: note = Synthetic: McolM67
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSKQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAEFRRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 34 - protein, 400 aa, synthetic construct
  REGION 1..400: note = Synthetic: McolM68
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYKTTPHWFDRLDEGNIEGGDVFIYNNDTLVIGVSERTNKEAILTIAKNIVNNKEAEFRRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 35 - protein, 400 aa, synthetic construct
  REGION 1..400: note = Synthetic: McolM69
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNKDTLVIGVSERTNKEAILTIAKNIVNNKEAEFRRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 36 - protein, 400 aa, synthetic construct
  REGION 1..400: note = Synthetic: McolM70
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKKIVNNKEAEFRRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 37 - protein, 400 aa, synthetic construct
  REGION 1..400: note = Synthetic: McolM71
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIKNNKEAEFRRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 38 - protein, 400 aa, synthetic construct
  REGION 1..400: note = Synthetic: McolM72
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAEFKRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 39 - protein, 400 aa, synthetic construct
  REGION 1..400: note = Synthetic: McolM73
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAEFRKIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 40 - protein, 360 aa, synthetic construct
  REGION 1..360: note = Synthetic: McolM74
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAEFRRIVAINVPPMPNLMHLDTWLTMVDKDKFLYSPNMLSVLKVWEIDLSAEIEMVETNKPLADVLESIIGVKPVLIPIAGKGATQLDIDIETHFDGTNYLTIAPGVVVGYSRNIK

SEQ ID NO: 41 - protein, 400 aa, synthetic construct
  REGION 1..400: note = Synthetic: McolM75
SKINVYSEIGELKEVLVHTPGDEIRRISPSRLDELLFSAILEPNEAIKEHKGFLKILQDKGIKVIQLSDLVAETYTYHATQKEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKKIVNNKEAEFRRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 42 - protein, 400 aa, synthetic construct
  REGION 1..400: note = Synthetic: McolM76
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSKQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKKIVNNKEAEFRRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 43 - protein, 400 aa, synthetic construct
  REGION 1..400: note = Synthetic: McolM77
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKKIVNNKEAKFRRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 44 - protein, 400 aa, synthetic construct
  REGION 1..400: note = Synthetic: McolM78
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKKIVNNKEAEFKRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 45 - protein, 400 aa, synthetic construct
  REGION 1..400: note = Synthetic: McolM79
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKKIVNNKEAEFRKIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 46 - protein, 400 aa, synthetic construct
  REGION 1..400: note = Synthetic: McolM80
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKKIVNNKEAEFRRIVAINVPPMPNLMHLDTWLTMVDKDKFLYSPNMLSVLQVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 47 - protein, 400 aa, synthetic construct
  REGION 1..400: note = Synthetic: McolM81
SKINVYSEIGELKEVLVHTPGDEIRRISPSRLDELLFSAILEPNEAIKEHKGFLKILQDKGIKVIQLSDLVAETYTYHATQKEREAFIETWLDEAEPALTKDLRAKVRSYVLSKEGTPVAMVRTMMAGVSKQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKKIVNNKEAEFRRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 48 - protein, 400 aa, synthetic construct
  REGION 1..400: note = Synthetic: McolM82
SKINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSKQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKKIVNNKEAKFRRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 49 - protein, 400 aa, synthetic construct
  REGION 1..400: note = Synthetic: McolM83
SKINVYSEIGELKEVLVHTPGDEIRRISPSRLDELLFSAILEPNEAIKEHKGFLKILQDKGIKVIQLSDLVAETYTYHATQKEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKKIVNNKEAKFRRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 50 - protein, 400 aa, synthetic construct
  REGION 1..400: note = Synthetic: McolM84
SKINVYSEIGELKEVLVHTPGDEIRRISPSRLDELLFSAILEPNEAIKEHKGFLKILQDKGIKVIQLSDLVAETYTYHATQKEREAFIETWLDEAEPALTKDLRAKVRSYVLSKEGTPVAMVRTMMAGVSKQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKKIVNNKEAKFRRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 51 - protein, 400 aa, synthetic construct
  REGION 1..400: note = Synthetic: McolM85
SKINVYSEIGELKEVLVHTPGDEIRRISPSRLDELLFSAILEPNEAIKEHKGFLKILQDKGIKVIQLSDLVAETYTYHATQKEREAFIETWLDEAEPALTKDLRAKVRSYVLSKEGTPVAMVRTMMAGVSKQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKKIVNNKEAKFKRIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 52 - protein, 400 aa, synthetic construct
  REGION 1..400: note = Synthetic: McolM86
SKINVYSEIGELKEVLVHTPGDEIRRISPSRLDELLFSAILEPNEAIKEHKGFLKILQDKGIKVIQLSDLVAETYTYHATQKEREAFIETWLDEAEPALTKDLRAKVRSYVLSKEGTPVAMVRTMMAGVSKQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKKIVNNKEAKFKKIVAINVPPMPNLMHLDTWLTMVDKDKFLYSPNMLSVLQVWEIDLSAEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 53 - protein, 400 aa, synthetic construct
  REGION 1..400: note = Synthetic: McolM94
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYATTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 54 - protein, 400 aa, synthetic construct
  REGION 1..400: note = Synthetic: McolM95
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYRTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 55 - protein, 400 aa, synthetic construct
  REGION 1..400: note = Synthetic: McolM96
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYNTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 56 - protein, 400 aa, synthetic construct
  REGION 1..400: note = Synthetic: McolM97
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYDTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 57 - protein, 400 aa, synthetic construct
  REGION 1..400: note = Synthetic: McolM98
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYQTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 58 - protein, 400 aa, synthetic construct
  REGION 1..400: note = Synthetic: McolM99
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYETTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 59 - protein, 400 aa, synthetic construct
  REGION 1..400: note = Synthetic: McolM100
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYGTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 60 - protein, 400 aa, synthetic construct
  REGION 1..400: note = Synthetic: McolM101
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYHTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 61 - protein, 400 aa, synthetic construct
  REGION 1..400: note = Synthetic: McolM102
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYITTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 62 - protein, 400 aa, synthetic construct
  REGION 1..400: note = Synthetic: McolM103
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYLTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 63 - protein, 400 aa, synthetic construct
  REGION 1..400: note = Synthetic: McolM104
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYMTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 64 - protein, 400 aa, synthetic construct
  REGION 1..400: note = Synthetic: McolM105
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYSTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 65 - protein, 400 aa, synthetic construct
  REGION 1..400: note = Synthetic: McolM106
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYTTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 66 - protein, 400 aa, synthetic construct
  REGION 1..400: note = Synthetic: McolM107
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLAVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 67 - protein, 400 aa, synthetic construct
  REGION 1..400: note = Synthetic: McolM108
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLRVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 68 - protein, 400 aa, synthetic construct
  REGION 1..400: note = Synthetic: McolM109
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLNVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 69 - protein, 400 aa, synthetic construct
  REGION 1..400: note = Synthetic: McolM110
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLDVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 70 - protein, 400 aa, synthetic construct
  REGION 1..400: note = Synthetic: McolM111
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLEVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 71 - protein, 400 aa, synthetic construct
  REGION 1..400: note = Synthetic: McolM112
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLGVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 72 - protein, 400 aa, synthetic construct
  REGION 1..400: note = Synthetic: McolM113
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLHVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 73 - protein, 400 aa, synthetic construct
  REGION 1..400: note = Synthetic: McolM114
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLIVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 74 - protein, 400 aa, synthetic construct
  REGION 1..400: note = Synthetic: McolM115
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLLVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 75 - protein, 400 aa, synthetic construct
  REGION 1..400: note = Synthetic: McolM116
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLMVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 76 - protein, 400 aa, synthetic construct
  REGION 1..400: note = Synthetic: McolM117
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLSVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 77 - protein, 400 aa, synthetic construct
  REGION 1..400: note = Synthetic: McolM118
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLTVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 78 - protein, 400 aa, synthetic construct
  REGION 1..400: note = Synthetic: McolM119
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLVVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 79 - protein, 400 aa, synthetic construct
  REGION 1..400: note = Synthetic: McolM120
SGINVYSEIGELKEVLVHTPGDEIRRISPSRLDELLFSAILEPNEAIKEHKGFLKILQDKGIKVIQLSDLVAETYTYHATQSEREAFIEAWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 80 - protein, 400 aa, synthetic construct
  REGION 1..400: note = Synthetic: McolM121
SGINVYSEIGELKEVLVHTPGDEIRRISPSRLDELLFSAILEPNEAIKEHKGFLKILQDKGIKVIQLSDLVAETYTYHATQSEREAFIERWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 81 - protein, 400 aa, synthetic construct
  REGION 1..400: note = Synthetic: McolM122
SGINVYSEIGELKEVLVHTPGDEIRRISPSRLDELLFSAILEPNEAIKEHKGFLKILQDKGIKVIQLSDLVAETYTYHATQSEREAFIENWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 82 - protein, 400 aa, synthetic construct
  REGION 1..400: note = Synthetic: McolM123
SGINVYSEIGELKEVLVHTPGDEIRRISPSRLDELLFSAILEPNEAIKEHKGFLKILQDKGIKVIQLSDLVAETYTYHATQSEREAFIEDWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 83 - protein, 400 aa, synthetic construct
  REGION 1..400: note = Synthetic: McolM124
SGINVYSEIGELKEVLVHTPGDEIRRISPSRLDELLFSAILEPNEAIKEHKGFLKILQDKGIKVIQLSDLVAETYTYHATQSEREAFIEQWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 84 - protein, 400 aa, synthetic construct
  REGION 1..400: note = Synthetic: McolM125
SGINVYSEIGELKEVLVHTPGDEIRRISPSRLDELLFSAILEPNEAIKEHKGFLKILQDKGIKVIQLSDLVAETYTYHATQSEREAFIEEWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 85 - protein, 400 aa, synthetic construct
  REGION 1..400: note = Synthetic: McolM126
SGINVYSEIGELKEVLVHTPGDEIRRISPSRLDELLFSAILEPNEAIKEHKGFLKILQDKGIKVIQLSDLVAETYTYHATQSEREAFIEGWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 86 - protein, 400 aa, synthetic construct
  REGION 1..400: note = Synthetic: McolM127
SGINVYSEIGELKEVLVHTPGDEIRRISPSRLDELLFSAILEPNEAIKEHKGFLKILQDKGIKVIQLSDLVAETYTYHATQSEREAFIEHWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 87 - protein, 400 aa, synthetic construct
  REGION 1..400: note = Synthetic: McolM128
SGINVYSEIGELKEVLVHTPGDEIRRISPSRLDELLFSAILEPNEAIKEHKGFLKILQDKGIKVIQLSDLVAETYTYHATQSEREAFIEIWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 88 - protein, 400 aa, synthetic construct
  REGION 1..400: note = Synthetic: McolM129
SGINVYSEIGELKEVLVHTPGDEIRRISPSRLDELLFSAILEPNEAIKEHKGFLKILQDKGIKVIQLSDLVAETYTYHATQSEREAFIELWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 89 - protein, 400 aa, synthetic construct
  REGION 1..400: note = Synthetic: McolM130
SGINVYSEIGELKEVLVHTPGDEIRRISPSRLDELLFSAILEPNEAIKEHKGFLKILQDKGIKVIQLSDLVAETYTYHATQSEREAFIEMWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 90 - protein, 400 aa, synthetic construct
  REGION 1..400: note = Synthetic: McolM131
SGINVYSEIGELKEVLVHTPGDEIRRISPSRLDELLFSAILEPNEAIKEHKGFLKILQDKGIKVIQLSDLVAETYTYHATQSEREAFIESWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 91 - protein, 400 aa, synthetic construct
  REGION 1..400: note = Synthetic: McolM132
SGINVYSEIGELKEVLVHTPGDEIRRISPSRLDELLFSAILEPNEAIKEHKGFLKILQDKGIKVIQLSDLVAETYTYHATQSEREAFIEVWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 92 - protein, 400 aa, synthetic construct
  REGION 1..400: note = Synthetic: McolM133
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVASYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 93 - protein, 400 aa, synthetic construct
  REGION 1..400: note = Synthetic: McolM134
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVNSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 94 - protein, 400 aa, synthetic construct
  REGION 1..400: note = Synthetic: McolM135
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVDSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 95 - protein, 400 aa, synthetic construct
  REGION 1..400: note = Synthetic: McolM136
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVQSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 96 - protein, 400 aa, synthetic construct
  REGION 1..400: note = Synthetic: McolM137
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVESYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 97 - protein, 400 aa, synthetic construct
  REGION 1..400: note = Synthetic: McolM138
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVGSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 98 - protein, 400 aa, synthetic construct
  REGION 1..400: note = Synthetic: McolM139
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVHSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 99 - protein, 400 aa, synthetic construct
  REGION 1..400: note = Synthetic: McolM140
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVISYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 100 - protein, 400 aa, synthetic construct
  REGION 1..400: note = Synthetic: McolM141
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVLSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 101 - protein, 400 aa, synthetic construct
  REGION 1..400: note = Synthetic: McolM142
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVMSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 102 - protein, 400 aa, synthetic construct
  REGION 1..400: note = Synthetic: McolM143
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVSSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 103 - protein, 400 aa, synthetic construct
  REGION 1..400: note = Synthetic: McolM144
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVTSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 104 - protein, 400 aa, synthetic construct
  REGION 1..400: note = Synthetic: McolM145
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVVSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 105 - protein, 400 aa, synthetic construct
  REGION 1..400: note = Synthetic: McolM146
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNADTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 106 - protein, 400 aa, synthetic construct
  REGION 1..400: note = Synthetic: McolM147
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNRDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 107 - protein, 400 aa, synthetic construct
  REGION 1..400: note = Synthetic: McolM148
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNDDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 108 - protein, 400 aa, synthetic construct
  REGION 1..400: note = Synthetic: McolM149
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNQDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 109 - protein, 400 aa, synthetic construct
  REGION 1..400: note = Synthetic: McolM150
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNEDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 110 - protein, 400 aa, synthetic construct
  REGION 1..400: note = Synthetic: McolM151
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNGDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 111 - protein, 400 aa, synthetic construct
  REGION 1..400: note = Synthetic: McolM152
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNHDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 112 - protein, 400 aa, synthetic construct
  REGION 1..400: note = Synthetic: McolM153
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNIDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 113 - protein, 400 aa, synthetic construct
  REGION 1..400: note = Synthetic: McolM154
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNLDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 114 - protein, 400 aa, synthetic construct
  REGION 1..400: note = Synthetic: McolM155
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNMDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 115 - protein, 400 aa, synthetic construct
  REGION 1..400: note = Synthetic: McolM156
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNSDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 116 - protein, 400 aa, synthetic construct
  REGION 1..400: note = Synthetic: McolM157
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNTDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 117 - protein, 400 aa, synthetic construct
  REGION 1..400: note = Synthetic: McolM158
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNVDTLVIGVSERTNKEAILTIAKNIVNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 118 - protein, 400 aa, synthetic construct
  REGION 1..400: note = Synthetic: McolM159
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIANNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 119 - protein, 400 aa, synthetic construct
  REGION 1..400: note = Synthetic: McolM160
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIRNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 120 - protein, 400 aa, synthetic construct
  REGION 1..400: note = Synthetic: McolM161
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNINNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 121 - protein, 400 aa, synthetic construct
  REGION 1..400: note = Synthetic: McolM162
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIDNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 122 - protein, 400 aa, synthetic construct
  REGION 1..400: note = Synthetic: McolM163
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIQNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 123 - protein, 400 aa, synthetic construct
  REGION 1..400: note = Synthetic: McolM164
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIENNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 124 - protein, 400 aa, synthetic construct
  REGION 1..400: note = Synthetic: McolM165
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIGNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 125 - protein, 400 aa, synthetic construct
  REGION 1..400: note = Synthetic: McolM166
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIHNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 126 - protein, 400 aa, synthetic construct
  REGION 1..400: note = Synthetic: McolM167
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIINNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 127 - protein, 400 aa, synthetic construct
  REGION 1..400: note = Synthetic: McolM168
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNILNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 128 - protein, 400 aa, synthetic construct
  REGION 1..400: note = Synthetic: McolM169
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIMNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 129 - protein, 400 aa, synthetic construct
  REGION 1..400: note = Synthetic: McolM170
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNISNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 130 - protein, 400 aa, synthetic construct
  REGION 1..400: note = Synthetic: McolM171
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNITNNKEAKFRRIVAINVPPMPNLMHLDTWLTMVDKDKFLYSPNMLSVLQVWEIDLSAEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 131 - protein, 400 aa, synthetic construct
  REGION 1..400: note = Synthetic: McolM172
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSREIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 132 - protein, 400 aa, synthetic construct
  REGION 1..400: note = Synthetic: McolM173
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSNEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 133 - protein, 400 aa, synthetic construct
  REGION 1..400: note = Synthetic: McolM174
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSDEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 134 - protein, 400 aa, synthetic construct
  REGION 1..400: note = Synthetic: McolM175
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSQEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 135 - protein, 400 aa, synthetic construct
  REGION 1..400: note = Synthetic: McolM176
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSEEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 136 - protein, 400 aa, synthetic construct
  REGION 1..400: note = Synthetic: McolM177
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSGEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 137 - protein, 400 aa, synthetic construct
  REGION 1..400: note = Synthetic: McolM178
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSHEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 138 - protein, 400 aa, synthetic construct
  REGION 1..400: note = Synthetic: McolM179
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSIEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 139 - protein, 400 aa, synthetic construct
  REGION 1..400: note = Synthetic: McolM180
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSLEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 140 - protein, 400 aa, synthetic construct
  REGION 1..400: note = Synthetic: McolM181
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSMEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 141 - protein, 400 aa, synthetic construct
  REGION 1..400: note = Synthetic: McolM182
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSSEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 142 - protein, 400 aa, synthetic construct
  REGION 1..400: note = Synthetic: McolM183
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSTEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 143 - protein, 400 aa, synthetic construct
  REGION 1..400: note = Synthetic: McolM184
SGINVYSEIGELKEVLVHTPGDEIRRISPSRLDELLFSAILEPNEAIKEHKGFLKILQDKGIKVIQLSDLVAETYTYHATQSEREAFIETWLDEAEPALTKDLRAKVRSYVLSKEGTPVAMVRTMMAGVSRQELNVESETELVVDPMPNLYFTRDPFASAGNGISLNNMKYVTRKRETIFAEFIFATHPDYVTTPHWFDRLDEGNIEGGDVFIYNNDTLVIGVSERTNKEAILTIAKNIVNNKEAKFRRIVAINVPPMPNLMHLDTWLTMVDKDKFLYSPNMLSVLQVWEIDLSVEIEMVETNKPLADVLESIIGVRPVLIPIAGAGATQLDIDIETHFDGTNYLTIAPGVVVGYSRNIKTEAALRAAGVTVLSFEGNQLSLGMGSARCMSMPLVREDVA

SEQ ID NO: 144 - protein, 400 aa, synthetic construct
  REGION 1..400: note = Synthetic: McolM188
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVASYVLSKEGTPVAMVRTMMAGVSKQELNVESETELVVDPMPNLYFTRDPFASAGNGISLNNMKYVTRKRETIFAEFIFATHPDYGTTPHWFDRLDEGNIEGGDVFIYNVDTLVIGVSERTNKEAILTIAKKIDNNKEAEFKKIVAINVPPMPNLMHLDTWLTMVDKDKFLYSPNMLSVLAVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 145 - protein, 400 aa, synthetic construct
  REGION 1..400: note = Synthetic: McolM189
SKINVYSEIGELKEVLVHTPGDEIRRISPSRLDELLFSAILEPNEAIKEHKGFLKILQDKGIKVIQLSDLVAETYTYHATQKEREAFIEKWLDEAEPALTKDLRAKVASYVLSKEGTPVAMVRTMMAGVSKQELNVESETELVVDPMPNLYFTRDPFASAGNGISLNNMKYVTRKRETIFAEFIFATHPDYGTTPHWFDRLDEGNIEGGDVFIYNVDTLVIGVSERTNKEAILTIAKKIDNNKEAEFKKIVAINVPPMPNLMHLDTWLTMVDKDKFLYSPNMLSVLAVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 146 - protein, 400 aa, synthetic construct
  REGION 1..400: note = Synthetic: McolM190
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYGTTPHWFDRLDEGNIEGGDVFIYNVDTLVIGVSERTNKEAILTIAKKIDNNKEAEFKKIVAINVPPMPNLMHLDTWLTMVDKDKFLYSPNMLSVLAVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 147 - protein, 400 aa, synthetic construct
  REGION 1..400: note = Synthetic: McolM191
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVASYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNVDTLVIGVSERTNKEAILTIAKKIDNNKEAEFKKIVAINVPPMPNLMHLDTWLTMVDKDKFLYSPNMLSVLAVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 148 - protein, 400 aa, synthetic construct
  REGION 1..400: note = Synthetic: McolM192
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVASYVLSKEGTPVAMVRTMMAGVSKQELNVESETELVVDPMPNLYFTRDPFASAGNGISLNNMKYVTRKRETIFAEFIFATHPDYGTTPHWFDRLDEGNIEGGDVFIYNKDTLVIGVSERTNKEAILTIAKKIDNNKEAEFKKIVAINVPPMPNLMHLDTWLTMVDKDKFLYSPNMLSVLAVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 149 - protein, 400 aa, synthetic construct
  REGION 1..400: note = Synthetic: McolM193
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVASYVLSKEGTPVAMVRTMMAGVSKQELNVESETELVVDPMPNLYFTRDPFASAGNGISLNNMKYVTRKRETIFAEFIFATHPDYGTTPHWFDRLDEGNIEGGDVFIYNVDTLVIGVSERTNKEAILTIAKKIKNNKEAEFKKIVAINVPPMPNLMHLDTWLTMVDKDKFLYSPNMLSVLAVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 150 - protein, 400 aa, synthetic construct
  REGION 1..400: note = Synthetic: McolM194
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVASYVLSKEGTPVAMVRTMMAGVSKQELNVESETELVVDPMPNLYFTRDPFASAGNGISLNNMKYVTRKRETIFAEFIFATHPDYGTTPHWFDRLDEGNIEGGDVFIYNVDTLVIGVSERTNKEAILTIAKKIDNNKEAEFKKIVAINVPPMPNLMHLDTWLTMVDKDKFLYSPNMLSVLKVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 151 - protein, 400 aa, synthetic construct
  REGION 1..400: note = Synthetic: McolM195
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVASYVLSKEGTPVAMVRTMMAGVSKQELNVESETELVVDPMPNLYFTRDPFASAGNGISLNNMKYVTRKRETIFAEFIFATHPDYGTTPHWFDRLDEGNIEGGDVFIYNVDTLVIGVSERTNKEAILTIAKKIDNNKEAEFKKIVAINVPPMPNLMHLDTWLTMVDKDKFLYSPNMLSVLAVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 152 - protein, 400 aa, synthetic construct
  REGION 1..400: note = Synthetic: McolM196
SKINVYSEIGELKEVLVHTPGDEIRRISPSRLDELLFSAILEPNEAIKEHKGFLKILQDKGIKVIQLSDLVAETYTYHATQKEREAFIEKWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNKDTLVIGVSERTNKEAILTIAKKIKNNKEAEFKKIVAINVPPMPNLMHLDTWLTMVDKDKFLYSPNMLSVLKVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 153 - protein, 400 aa, synthetic construct
  REGION 1..400: note = Synthetic: McolM197
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNKDTLVIGVSERTNKEAILTIAKKIKNNKEAEFKKIVAINVPPMPNLMHLDTWLTMVDKDKFLYSPNMLSVLKVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 154 - protein, 400 aa, synthetic construct
  REGION 1..400: note = Synthetic: McolM198
SKINVYSEIGELKEVLVHTPGDEIRRISPSRLDELLFSAILEPNEAIKEHKGFLKILQDKGIKVIQLSDLVAETYTYHATQKEREAFIEKWLDEAEPALTKDLRAKVASYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNKDTLVIGVSERTNKEAILTIAKKIKNNKEAEFKKIVAINVPPMPNLMHLDTWLTMVDKDKFLYSPNMLSVLKVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 155 - protein, 400 aa, synthetic construct
  REGION 1..400: note = Synthetic: McolM199
SKINVYSEIGELKEVLVHTPGDEIRRISPSRLDELLFSAILEPNEAIKEHKGFLKILQDKGIKVIQLSDLVAETYTYHATQKEREAFIEKWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYGTTPHWFDRLDEGNIEGGDVFIYNKDTLVIGVSERTNKEAILTIAKKIKNNKEAEFKKIVAINVPPMPNLMHLDTWLTMVDKDKFLYSPNMLSVLKVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 156 - protein, 400 aa, synthetic construct
  REGION 1..400: note = Synthetic: McolM200
SKINVYSEIGELKEVLVHTPGDEIRRISPSRLDELLFSAILEPNEAIKEHKGFLKILQDKGIKVIQLSDLVAETYTYHATQKEREAFIEKWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNVDTLVIGVSERTNKEAILTIAKKIKNNKEAEFKKIVAINVPPMPNLMHLDTWLTMVDKDKFLYSPNMLSVLKVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 157 - protein, 400 aa, synthetic construct
  REGION 1..400: note = Synthetic: McolM201
SKINVYSEIGELKEVLVHTPGDEIRRISPSRLDELLFSAILEPNEAIKEHKGFLKILQDKGIKVIQLSDLVAETYTYHATQKEREAFIEKWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNKDTLVIGVSERTNKEAILTIAKKIDNNKEAEFKKIVAINVPPMPNLMHLDTWLTMVDKDKFLYSPNMLSVLKVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 158 - protein, 400 aa, synthetic construct
  REGION 1..400: note = Synthetic: McolM202
SKINVYSEIGELKEVLVHTPGDEIRRISPSRLDELLFSAILEPNEAIKEHKGFLKILQDKGIKVIQLSDLVAETYTYHATQKEREAFIEKWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNKDTLVIGVSERTNKEAILTIAKKIKNNKEAEFKKIVAINVPPMPNLMHLDTWLTMVDKDKFLYSPNMLSVLAVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 159 - protein, 400 aa, synthetic construct
  REGION 1..400: note = Synthetic: McolM203
SKINVYSEIGELKEVLVHTPGDEIRRISPSRLDELLFSAILEPNEAIKEHKGFLKILQDKGIKVIQLSDLVAETYTYHATQKEREAFIEKWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNKDTLVIGVSERTNKEAILTIAKKIKNNKEAEFKKIVAINVPPMPNLMHLDTWLTMVDKDKFLYSPNMLSVLKVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 160 - protein, 400 aa, synthetic construct
  REGION 1..400: note = Synthetic: McolM204
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVASYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNKDTLVIGVSERTNKEAILTIAKKIKNNKEAEFKKIVAINVPPMPNLMHLDTWLTMVDKDKFLYSPNMLSVLKVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 161 - protein, 400 aa, synthetic construct
  REGION 1..400: note = Synthetic: McolM205
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYGTTPHWFDRLDEGNIEGGDVFIYNKDTLVIGVSERTNKEAILTIAKKIKNNKEAEFKKIVAINVPPMPNLMHLDTWLTMVDKDKFLYSPNMLSVLKVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 162 - protein, 400 aa, synthetic construct
  REGION 1..400: note = Synthetic: McolM206
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNVDTLVIGVSERTNKEAILTIAKKIKNNKEAEFKKIVAINVPPMPNLMHLDTWLTMVDKDKFLYSPNMLSVLKVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 163 - protein, 400 aa, synthetic construct
  REGION 1..400: note = Synthetic: McolM207
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNKDTLVIGVSERTNKEAILTIAKKIDNNKEAEFKKIVAINVPPMPNLMHLDTWLTMVDKDKFLYSPNMLSVLKVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 164 - protein, 400 aa, synthetic construct
  REGION 1..400: note = Synthetic: McolM208
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNKDTLVIGVSERTNKEAILTIAKKIKNNKEAEFKKIVAINVPPMPNLMHLDTWLTMVDKDKFLYSPNMLSVLAVWEIDLSKEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 165 - protein, 400 aa, synthetic construct
  REGION 1..400: note = Synthetic: McolM209
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNKDTLVIGVSERTNKEAILTIAKKIKNNKEAEFKKIVAINVPPMPNLMHLDTWLTMVDKDKFLYSPNMLSVLKVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 166 - protein, 400 aa, synthetic construct
  REGION 1..400: note = Synthetic: McolM210
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVASYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNKDTLVIGVSERTNKEAILTIAKKIKNNKEAEFKKIVAINVPPMPNLMHLDTWLTMVDKDKFLYSPNMLSVLKVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 167 - protein, 400 aa, synthetic construct
  REGION 1..400: note = Synthetic: McolM211
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYGTTPHWFDRLDEGNIEGGDVFIYNKDTLVIGVSERTNKEAILTIAKKIKNNKEAEFKKIVAINVPPMPNLMHLDTWLTMVDKDKFLYSPNMLSVLKVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 168 - protein, 400 aa, synthetic construct
  REGION 1..400: note = Synthetic: McolM212
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNVDTLVIGVSERTNKEAILTIAKKIKNNKEAEFKKIVAINVPPMPNLMHLDTWLTMVDKDKFLYSPNMLSVLKVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 169 - protein, 400 aa, synthetic construct
  REGION 1..400: note = Synthetic: McolM213
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNKDTLVIGVSERTNKEAILTIAKKIDNNKEAEFKKIVAINVPPMPNLMHLDTWLTMVDKDKFLYSPNMLSVLKVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 170 - protein, 400 aa, synthetic construct
  REGION 1..400: note = Synthetic: McolM214
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVKSYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNKDTLVIGVSERTNKEAILTIAKKIKNNKEAEFKKIVAINVPPMPNLMHLDTWLTMVDKDKFLYSPNMLSVLAVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 171 - protein, 400 aa, synthetic construct
  REGION 1..400: note = Synthetic: McolM215
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVASYVLSKEGTPVAMVRTMMAGVSKQELNVESETELVVDPMPNLYFTRDPFASAGNGISLNNMKYVTRKRETIFAEFIFATHPDYGTTPHWFDRLDEGNIEGGDVFIYNKDTLVIGVSERTNKEAILTIAKKIKNNKEAEFKKIVAINVPPMPNLMHLDTWLTMVDKDKFLYSPNMLSVLKVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 172 - protein, 400 aa, synthetic construct
  REGION 1..400: note = Synthetic: McolM216
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVASYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNVDTLVIGVSERTNKEAILTIAKKIKNNKEAEFKKIVAINVPPMPNLMHLDTWLTMVDKDKFLYSPNMLSVLKVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 173 - protein, 400 aa, synthetic construct
  REGION 1..400: note = Synthetic: McolM217
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVASYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNKDTLVIGVSERTNKEAILTIAKKIDNNKEAEFKKIVAINVPPMPNLMHLDTWLTMVDKDKFLYSPNMLSVLKVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 174 - protein, 400 aa, synthetic construct
  REGION 1..400: note = Synthetic: McolM218
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVASYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNKDTLVIGVSERTNKEAILTIAKKIKNNKEAEFKKIVAINVPPMPNLMHLDTWLTMVDKDKFLYSPNMLSVLAVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 175 - protein, 400 aa, synthetic construct
  REGION 1..400: note = Synthetic: McolM219
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVASYVLSKEGTPVAMVRTMMAGVSKQELNVESETELVVDPMPNLYFTRDPFASAGNGISLNNMKYVTRKRETIFAEFIFATHPDYGTTPHWFDRLDEGNIEGGDVFIYNKDTLVIGVSERTNKEAILTIAKKIKNNKEAEFKKIVAINVPPMPNLMHLDTWLTMVDKDKFLYSPNMLSVLAVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 176 - protein, 400 aa, synthetic construct
  REGION 1..400: note = Synthetic: McolM220
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVASYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNVDTLVIGVSERTNKEAILTIAKKIKNNKEAEFKKIVAINVPPMPNLMHLDTWLTMVDKDKFLYSPNMLSVLAVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 177 - protein, 400 aa, synthetic construct
  REGION 1..400: note = Synthetic: McolM221
SKINVYSEIGELKEVLVHTPGDEIRRISPSRLDELLFSAILEPNEAIKEHKGFLKILQDKGIKVIQLSDLVAETYTYHATQKEREAFIEVWLDEAEPALTKDLRAKVASYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNKDTLVIGVSERTNKEAILTIAKKIDNNKEAEFKKIVAINVPPMPNLMHLDTWLTMVDKDKFLYSPNMLSVLAVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK

SEQ ID NO: 178 - protein, 400 aa, synthetic construct
  REGION 1..400: note = Synthetic: McolM222
SKINVYSEIGELKEVLVHTPGDEIRRISPSRLDELLFSAILEPNEAIKEHKGFLKILQDKGIKVIQLSDLVAETYTYHATQKEREAFIEKWLDEAEPALTKDLRAKVASYVLSKEGTPVAMVRTMMAGVSKQELNVESETELVVDPMPNLYFTRDPFASAGNGISLNNMKYVTRKRETIFAEFIFATHPDYKTTPHWFDRLDEGNIEGGDVFIYNVDTLVIGVSERTNKEAILTIAKKIKNNKEAEFKKIVAINVPPMPNLMHLDTWLTMVDKDKFLYSPNMLSVLKVWEIDLSIEIEMVETNKPLADVLESIIGVKPVLIPIAGKGATQLDIDIETHFDGTNYLTIAPGVVVGYSRNIKTEAALRAAGVTVLSFEGNQLSLGMGSARCMSMPLVREDVK